(19) United States
(12) Patent Application Publication
(10) Pub. No.: US 2009/0105217 A1
(43) Pub. Date: Apr. 23, 2009

(54) 2-PHENYL-5-AMINO-1,3,4-OXADIAZOLES AND THEIR USE AS NICOTINIC ACETYLCHOLINE RECEPTOR LIGANDS

(76) Inventors: Frank Teen Coppo, Collegeville, PA (US); Emma S.L. Maskell, Essex (GB); Sally Redshaw, Essex (GB); John Skidmore, Essex (GB); Robert William Ward, Essex (GB); David Matthew Wilson, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/302,054

(22) PCT Filed: May 29, 2007
(86) PCT No.: PCT/EP07/55159

§ 371 Date: Nov. 24, 2008

(51) Int. Cl. A61K 31/553; C07D 413/12; A61K 31/5377; A61K 31/4245; A61P 25/00; A61P 29/00; A61K 31/454; A61P 3/04; A61K 31/55; A61K 31/551; A61P 31/04; A61P 1/00

(52) U.S. Cl. 514/211.15; 544/138; 514/236.2; 514/364; 548/143; 546/209; 514/326; 540/603; 514/217.1; 514/221; 540/556; 540/544

(57) ABSTRACT

Novel oxadiazole derivatives of formula (I) having pharmacological activity, processes for their preparation, compositions containing them and their use in the treatment of neurological, psychiatric disorders and gastrointestinal disorders through modulation of the nicotinic α7 receptor.

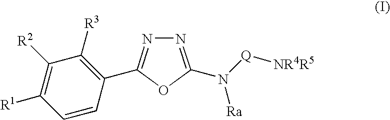

The present invention relates to novel oxadiazole derivatives having pharmacological activity, processes for their preparation, compositions containing them and their use in the treatment of neurological, psychiatric disorders and gastrointestinal disorders.

Nicotinic acetylcholine receptors (nAChRs) are cation-specific, excitatory ligand-gated ion channels that are widely expressed throughout the central and peripheral nervous systems. To date, 16 mammalian nAChR subunit genes have been cloned: 5 encoding muscle receptor subunits, and 11 encoding neuronal receptor subunits. The nicotinic α7 receptor subunit is predominantly expressed in the mammalian central nervous system (CNS), where it is thought to assemble as a functional homopentameric complex, and is also expressed in peripheral tissues including the sympathetic nervous system, immune cells and the GI tract. Activation of neuronal nicotinic α7 receptors by selective agonists or the endogenous ligand acetylcholine can modulate the release of various neurotransmitters including glutamate, GABA, dopamine, and noradrenaline and, thus, has the potential to modulate a range of neurological functions. Additionally, in vivo studies have shown that α7 nAChR agonists can modulate neurotransmitter release in brain areas such as the cortex and hippocampus that are relevant to cognition (Paterson D et al, (2000) Prog Neurobiol 61:75-111.

A number of literature reports have demonstrated the cognitive enhancing properties of α7 nAChR agonists (e.g. GTS-21 (3-(2,4-dimethoxybenzylidene)anabaseine), AR-R 17779 ((−)-spiro[1-azabicyclo[2.2.2]octane-3,5′-oxazoldin-2′-one] 4-propyl-benzylidene anabaseine) and SSR-180771 (4-bromophenyl 1,4-diazabicyclo[3.2.2]nonane-4-carboxylate hydrochloride) in rodent and primate cognition models including the radial arm maze (Levin E. D. et al. (1999), Behavioural Pharmacology. 10(6-7):675-80), social recognition (Van Kampen M. et al. (2004) Psychopharmacology. 172(4):375-83), elevated plus maze/delayed matching-to-sample test (Briggs C. A. et al. (1997) Pharmacology, Biochemistry & Behavior. 57(1-2):231-41), active avoidance and radial arm maze (Arendash G. W. et al. (1995) Brain Research. 674(2):252-9).

Consistent with these animal studies, recent data from small clinical trials demonstrates that the α7 nAChR partial agonist GTS-21 enhanced memory and attention in healthy volunteers (Kitagawa H. et al. (2003) Neuropsychopharmacology. 28(3):542-51). Furthermore, beneficial effects of nicotine on attention parameters have also been demonstrated in Alzheimer's disease (Potter A. and Levin E. D. (1997) Drugs & Aging. 11(3):206-28), age-associated memory impairments (White H. K. and Levin E. D. (2004), Psychopharmacology. 171(4):465-71) and attention deficit disorder (Levin E. D. et al. (1996) Psychopharmacology. 123(1):55-63). Activation of α7 nAChRs has also been reported to ameliorate sensory gating deficits in both preclinical (Simosky J. K. et al., (2001) Biological Psychiatry. 50(7):493-500) and small clinical studies. These data suggest that novel α7 nAChR agonists and/or partial agonists such as the current series could be useful for the treatment of cognitive impairments in neurological and psychiatric disorders such as Alzheimer's disease, related neurodegenerative disorders and schizophrenia.

WO 2004/014370 (AstraZeneca AB and NPS Pharmaceuticals, Inc) discloses compounds and their use in therapy. WO 2005/077373 (AstraZeneca AB and NPS Pharmaceuticals, Inc) discloses the use of certain compounds for the inhibition of transient lower esophageal sphincter relaxations and the use of certain compounds for the treatment of gastro-esophageal reflux disease. EP 560407 (Schering Corp) discloses compounds with pharmaceutical activity i.e antiviral activity. WO2005/087236 (Glaxo Group Limited) discloses muscarinic acetylcholine receptor antagonists and methods of using them. WO2002/055484 (Takeda Chemical Industries) discloses compounds to increase the amount of low-density lipoprotein receptor which are useful as blood lipid depressants.

The present invention provides, in a first aspect, compounds of formula (I):

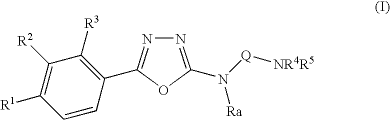

wherein
Q represents —(CH₂)ₙ— wherein n represents 3 or 4;
Ra represents hydrogen or CH₃;
R¹ represents —NHCOR⁷;
R² represents hydrogen, halogen or C₁₋₆alkyl;
R³ represents hydrogen, C₁₋₆alkyl, C₁₋₆alkoxy, halo, haloC₁₋₆alkyl, or —OhaloC₁₋₆alkyl;
R⁴ and R⁵ independently represent hydrogen, C₁₋₆ alkyl, C₃₋₆cycloalkyl, or a 5 to 6 membered heterocyclic ring which is unsubstituted or substituted with 1, 2, or 3 substituents selected from methyl or fluoro;
or R⁴ and R⁵ together with the nitrogen atom to which they are attached form a nitrogen containing heterocyclyl group which is unsubstituted or substituted with 1, 2, or 3 substituents selected from methyl or fluoro;
R⁷ represents C₁₋₅alkyl, C₃₋₈ cycloalkyl, tetrahydropyranyl, thiophenyl, pyridyl, oxazolyl, isoxazolyl, furanyl, —(CH₂)ₘaryl wherein the furanyl, thiophenyl, pyridyl, oxazolyl, isooxazolyl or aryl can be unsubstituted or substituted by one to three substitutents selected from halo, methyl, CF₃, OCF₃ or OMe wherein OMe is not present at the position ortho to the carbonyl and the CF₃ is not at the position meta or para to the carbonyl;
m represents 0 or 1;
with the proviso that R⁷ is not isopropyl;
and salts thereof.

N-[4-(5-{[3-(Diethylamino)propyl]amino}-1,3,4-oxadiazol-2-yl)phenyl]-4-(methyloxy)benzamide, N-(4-{5-[[3-(dimethylamino)propyl](methyl)amino]-1,3,4-oxadiazol-2-yl}phenyl)-2-fluorobenzamide, 2,2-dimethyl-N-[4-(5-{[3-(1-piperidinyl)propyl]amino}-1,3,4-oxadiazol-2-yl)phenyl]propanamide, N-[4-(5-{[3-(3,3-difluoro-1-piperidinyl)propyl]amino}-1,3,4-oxadiazol-2-yl)phenyl]-2-fluorobenzamide and N-[4-(5-{[3-(dipropylamino)propyl]amino}-1,3,4-oxadiazol-2-yl)phenyl]-2-fluorobenzamide were not found to have activity in the alpha 7 assay described in the Examples.

The term ‘C₁₋₆ alkyl’ as used herein as a group or a part of the group refers to a linear or branched saturated hydrocarbon group containing from 1 to 6 carbon atoms. Examples of such groups include methyl, ethyl, n-propyl, isopropyl, n-butyl, isobutyl, sec-butyl, tert-butyl, n-pentyl, isopentyl, neopentyl or hexyl and the like. Unless a particular structure is specified, the terms propyl, butyl etc include all straight and branched chain forms having the appropriate number of carbon atoms e.g. propyl includes n-propyl and isopropyl.

The term ‘C₁₋₆ alkoxy’ as used herein refers to an —O—C₁₋₆ alkyl group wherein C₁₋₆ alkyl is as defined herein. Examples of such groups include methoxy, ethoxy, propoxy, butoxy, pentoxy or hexoxy and the like. As for alkyl unless a particular structure is specified the terms propoxy, butoxy etc include all straight and branched chain forms having the appropriate number of carbon atoms e.g. propoxy includes n-propoxy and isopropoxy

The term ‘C₃₋₈ cycloalkyl’ as used herein refers to a saturated monocyclic hydrocarbon ring of 3 to 8 carbon atoms. Examples of such groups include cyclopropyl, cyclobutyl, cyclopentyl, cyclohexyl, cycloheptyl or cyclooctyl and the like.

The term ‘halogen’ as used herein refers to a fluorine, chlorine, bromine or iodine atom.

The term haloC₁₋₆alkyl as used herein refers to a C₁₋₆alkyl group substituted with one or more halogen groups, e.g CF₃, CF₂CH or CF₃CH₂.

The term ‘aryl’ as used herein refers to a C₆₋₁₂ monocyclic or bicyclic hydrocarbon ring wherein at least one ring is aromatic. Examples of such groups include phenyl, naphthyl or tetrahydronaphthyl and the like.

The term “nitrogen containing heterocyclyl group” includes a 4-7 membered monocyclic saturated or partially unsaturated aliphatic ring system, a 6-9 membered saturated or partially unsaturated bridged ring system or a 4-7 membered saturated or partially unsaturated aliphatic ring fused to a benzene ring containing a nitrogen atom in addition to 1 or 2 optional additional heteroatoms selected from oxygen, nitrogen or sulphur. Suitable examples of such monocyclic rings include pyrrolidinyl, azetidinyl, piperidinyl, piperazinyl, morpholinyl, hexahydroazepanyl, hexahydrodiazepanyl and homomorpholinyl. Examples of such bridged ring systems are azabicycloheptanyl and azabicyclononanyl. Suitable examples of benzofused heterocyclic rings include indolinyl, isoindolinyl, 2,3,4,5-tetrahydro-1H-3-benzazepinyl or tetrahydroisoquinolinyl.

In one embodiment Q represents (CH₂)ₙ wherein n is 3. In one embodiment Q represents (CH₂)ₙ wherein n is 4.

In one embodiment Ra represents hydrogen.

In one embodiment, R² represents hydrogen.

In one embodiment R³ represents hydrogen, chloro, fluoro, CF₃, —OCH₃ or ethyl.

In one embodiment R³ represents hydrogen, C₁₋₆ alkyl, C₁₋₆ alkoxy.

In one embodiment, R³ represents hydrogen or C₁₋₆ alkoxy (e.g. methoxy).

In one embodiment, R³ represents halo, for example fluoro or chloro.

In one embodiment, R⁴ and R⁵ together with the nitrogen atom which they are attached form an nitrogen containing heterocyclyl group e.g. piperidinyl, morpholinyl, pyrrolidinyl or 1,4-diazabicyclo[3.2.2]nonane wherein the heterocyclyl group is unsubstituted or substituted with 1, 2, or 3 substituents selected from methyl or fluoro.

In one embodiment R⁴ and R⁵ either both represent C₁₋₆ alkyl or R⁴ and R⁵ together with the nitrogen atom to which they are attached form a nitrogen containing heterocyclyl group wherein the heterocyclyl group is unsubstituted or substituted with 1, 2, or 3 substituents selected from methyl or fluoro

In one embodiment, R⁴ and R⁵ either both represent C₁₋₆ alkyl (e.g. ethyl) or R⁴ and R⁵ together with the nitrogen atom to which they are attached form a nitrogen containing heterocyclyl group (e.g. 1-piperidinyl, 4-morpholinyl, 1-pyrrolydinyl or 1-hexahydroazepinyl).

In one embodiment, R⁴ and R⁵ either both represent C₁₋₆ alkyl (e.g. ethyl) or R⁴ and R⁵ together with the nitrogen atom to which they are attached form a nitrogen containing heterocyclyl group e.g. 1-piperidinyl or 4-morpholinyl wherein the heterocyclyl group is unsubstituted or substituted with 1, 2, or 3 substituents selected from methyl or fluoro

In one embodiment, R⁴ and R⁵ together with the nitrogen atom which they are attached form a nitrogen containing heterocyclyl group (e.g. 1-piperidinyl or 4-morpholinyl) wherein the heterocyclyl group is unsubstituted or substituted with 1, 2, or 3 substituents selected from methyl or fluoro

In a further embodiment, R⁴ and R⁵ together with the nitrogen atom to which they are attached form 1-piperidinyl which is unsubstituted or substituted with 1, 2, or 3 substituents selected from methyl or fluoro. In a further embodiment, R⁴ and R⁵ together with the nitrogen atom to which they are attached form 4-morpholinyl.

In one embodiment R⁷ represents ethyl, propyl, isobutyl, C₃₋₈ cycloalkyl, or aryl wherein the aryl can be unsubstituted or substituted by one to three halogen atoms.

In one embodiment, R⁷ represents a cyclohexyl group.

In one embodiment, R⁷ represents aryl unsubstituted or substituted by a halogen atom (e.g. 2-fluorophenyl).

In one embodiment, R⁷ represents phenyl unsubstituted or substituted by a halogen atom (e.g. 2-fluorophenyl or 3-fluorophenyl).

The present invention also provides compounds of formula (Ia):

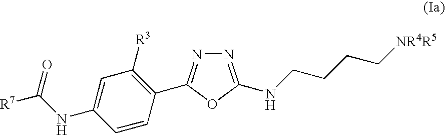

wherein
R³ represents hydrogen, chloro, fluoro, CF₃, OCH₃ or ethyl;
R⁴ and R⁵ together with the nitrogen atom to which they are attached form a nitrogen containing heterocyclyl group which is unsubstituted or substituted with 1, 2, or 3 substituents selected from methyl or fluoro;
R⁷ represents a cyclohexyl group or a phenyl which can be unsubstituted or substituted by one to three halo substitutents;
and salts thereof.

One specific set of compounds which may be mentioned are those wherein:

R² represents hydrogen, R¹ represents —NR⁶COR⁷ and R⁷ represents ethyl, propyl, isobutyl or C₃₋₈ cycloalkyl;
R³ represents C₁₋₆ alkyl or C₁₋₆ alkoxy; and
R⁴ and R⁵ both represent C₁₋₆ alkyl.

The present invention also provides compounds of formula (Ib)

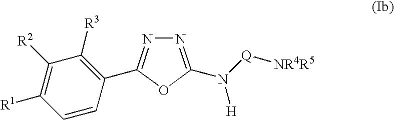

wherein
Q represents —(CH₂)ₙ— wherein n represents 3 or 4
R¹ represents —NR⁶COR⁷ and R² represents hydrogen, halogen or C₁₋₆alkyl;
R³ represents hydrogen, C₁₋₆ alkyl, C₁₋₆ alkoxy, halo, haloC₁₋₆alkyl, or —OCF₃;
R⁴ and R⁵ independently represent C₁₋₆ alkyl, C₃₋₆cycloalkyl or R⁴ and R⁵ together with the nitrogen atom to which they are attached form a nitrogen containing heterocyclyl group which is unsubstituted or substituted with 1, 2, or 3 substituents selected from methyl or fluoro;
R⁶ represents hydrogen;
R⁷ represents ethyl, propyl, butyl, pentyl, C₃₋₈ cycloalkyl, thiophenyl, pyridinyl, —(CH₂)ₘaryl wherein the aryl can be unsubstituted or substituted by one to three substitutents selected from halo, methyl or OMe wherein OMe is not present at the position ortho to the carbonyl
m represents 0 or 1;
and pharmaceutically acceptable salts thereof.

The present invention also provides compounds of formula (Ic) and pharmaceutically acceptable salts thereof:

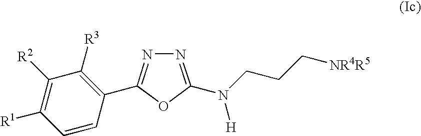

wherein
R¹ represents —NR⁶COR⁷;
R² represents hydrogen
R³ represents hydrogen, C₁₋₆ alkyl or C₁₋₆ alkoxy;
R⁴ and R⁵ either both represent C₁₋₆ alkyl or R⁴ and R⁵ together with the nitrogen atom to which they are attached form a nitrogen containing heterocyclyl group;
R⁶ represents hydrogen;
R⁷ represents ethyl, propyl, isobutyl, C₃₋₈ cycloalkyl or aryl optionally substituted by one or more halogen atoms.

Compounds of formula (I) include the compounds of Examples 1 to 237 and salts thereof.

In one embodiment compounds of formula (I) include the compounds of Examples 1 to 237 and pharmaceutically acceptable salts thereof.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives thereof (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H may afford certain therapeutic advantages resulting from greater metabolic stability, for example increased in vivo half-life or reduced dosage requirements and, hence, are considered useful in some circumstances.

The compounds of formula (I) may be prepared in crystalline or non-crystalline form, and may be optionally hydrated or solvated. This invention includes with in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

It will be appreciated that for compounds of formula (I) in all forms, crystalline or non crystalline, solvates or hydrates it may be possible to prepare the compounds as salts, preferably pharmaceutically acceptable salts. A further aspect of the invention is compounds of formula (I) and pharmaceutically acceptable salts thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like. In some circumstances some salts may be non-stoichiometric.

It will be understood by the skilled person that compounds of formula (I) covers pharmaceutically acceptable derivatives e.g. any pharmaceutically acceptable salt, or ester solvate, hydrate or solvate or hydrate of a salt of a compound of formula (I), or any other compound which upon administration to the recipient is capable of providing (directly or indirectly) a compound of formula (I), e.g a prodrug.

Compounds of formula (I) can be prepared as set forth in the following Schemes and in the Examples. The following processes form another aspect of the present invention.

The present invention also provides processes for the preparation of a compound of formula (I) or a salt thereof, which processes comprises:

(a) cyclising a compound of formula (II) or a protected derivative thereof

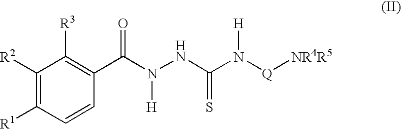

wherein Q, R¹, R², R³, R⁴ and R⁵ are as defined for compounds of formula (I);
(b) reacting a compound of formula (III) or a protected derivative thereof

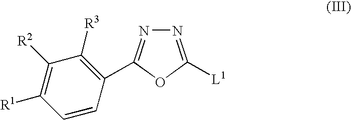

wherein R¹, R² and R³ are as defined for compounds of formula (I) and L¹ represents a suitable leaving group, such as a halogen atom (e.g. chlorine or bromine), —SO₂—C₁₋₆ alkyl, or —SO₂-benzyl group, with a compound of formula (IV):

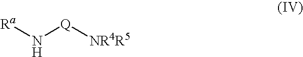

wherein Q, R^{a}, R⁴ and R⁵ are as defined for compounds of formula (I);
(c) reacting a compound of formula (V) or a protected derivative thereof

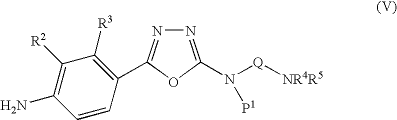

wherein Q, R², R³, R⁴, and R⁵ are as defined for compounds of formula (I), and P¹ is hydrogen or a protecting group for example a Boc group, with a compound of formula R⁷COL², wherein R⁷ is as defined for compounds of formula (I) and L² represents a suitable leaving group, such as a halogen atom (e.g. chlorine), or with a compound of formula R⁷COOH and a suiable coupling agent such as EDAC.HCl, optionally in the presence of HOBt or a base such as Et₃N, and deprotecting if necessary; or
(d) deprotecting a protected compound of formula (I); or
(e) interconversion of a compound of formula (I) to another compound of formula (I).

A further process of the invention is the preparation of pharmaceutically acceptable salts of compounds of formula (I).

Process (a) typically comprises the use of a suitable reagent, such as N-cyclohexylcarbodiimide, N′-methyl polystyrene (P-DCC) or EDAC.HCl in the presence of a suitable solvent, such as N,N-dimethylformamide at a suitable temperature, e.g. 80° C. Alternatively the cyclisation can be achieved by dissolving compound of formula (II) in IMS, cooling to about 0° C. adjusting the pH to about 9 using a suitable base such as NaOH and then treating with I₂/KI or TsCl/pyridine in a suitable solvent such as tetrahydrofuran.

Process (b) typically comprises the use of a suitable solvent, such as dioxan at a suitable temperature, such as 100° C. in a microwave, or at lower temperatures with a longer reaction time.

Process (c) typically comprises an acylation reaction in the presence of a suitable solvent, such as dichloromethane and a suitable base, such as N,N′-diisopropylethylamine or triethylamine. Where compounds of formula (I) are prepared using R⁷COOH, a suitable coupling reagent such as N-cyclohexylcarbodiimide, N′-methyl polystyrene or EDAC.HCl in combination with N-hydroxybenzotriazole (HOBt) is used.

In process (d), examples of protecting groups and the means for their removal can be found in T. W. Greene ‘Protective Groups in Organic Synthesis’ (J. Wiley and Sons, 3^{rd} Ed. 1999). Suitable amine protecting groups include acyl (e.g. acetyl) [removed by hydrolysis], carbamates (e.g. 2′,2′,2′-trichloroethoxycarbonyl) [removed with zinc in acetic acid], benzyloxycarbonyl [removed by acidolysis or hydrogenolysis] or t-butoxycarbonyl) [removed by acidolysis e.g. using an acid such as HCl or TFA] and arylalkyl (e.g. benzyl [removed by hydrogenolysis] as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

The term “protected derivative thereof” is used herein to refer to a compound which includes a protecting group, for example those referred to above.

Process (e) may be performed using conventional interconversion procedures such as epimerisation, oxidation, reduction, alkylation, nucleophilic or electrophilic aromatic substitution or amide bond formation.

Compounds of formula (II) may be prepared in accordance with the following Scheme 1:

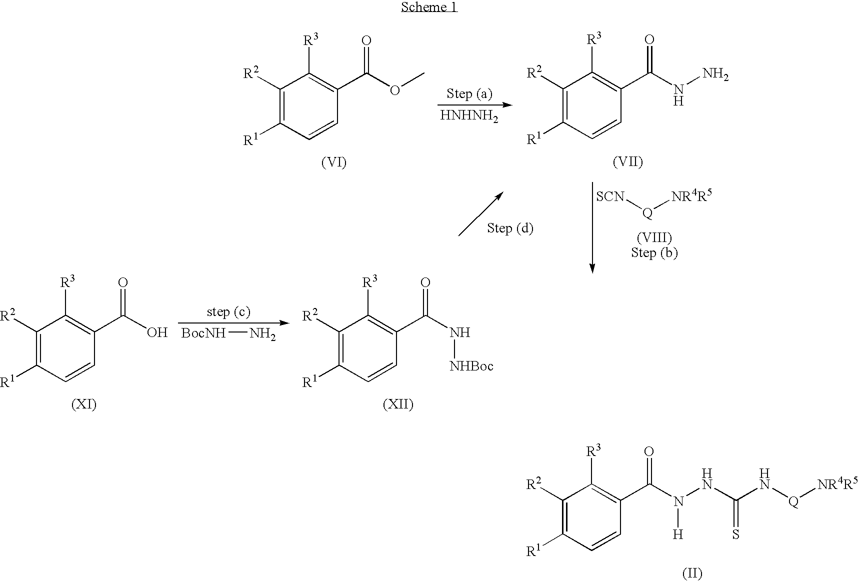

wherein Q, R¹, R², R³, R⁴ and R⁵ are as defined for compounds of formula (I).

Step (a) typically comprises the use of a suitable reagent, such as hydrazine monohydrate in the presence of a suitable solvent, such as methanol at a suitable temperature, e.g. from room temperature to reflux.

Step (b) typically comprises the reaction of a compound of formula (VII) with a compound of formula (VIII) in the presence of a suitable solvent, such as tetrahydrofuran at a suitable temperature, e.g. 60° C. room temperature.

Step (c) typically comprises the reaction of a compound of formula (XI) with BocNHNH₂ in a suitable solvent e.g. CH₂Cl₂ using a suitable coupling agent such as EDAC.HCl and optionally HOBt.

In Step (d) the protecting group such as a Boc group can be removed by conventional means.

It will be appreciated that Compounds of formula (I) may be prepared directly from compounds of formula (VII) in a one pot process by effecting the cyclisation without isolation of intermediate (II) using similar conditions to those described above.

Compounds of formula (VI) may be prepared in accordance with the following Scheme 2 using compound of formula (Xa) or (Xb):

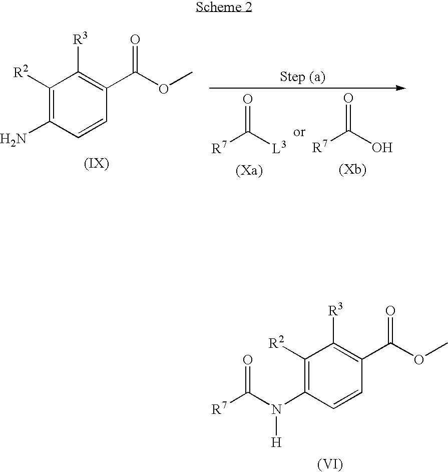

wherein R², R³, and R⁷ are as defined for compounds of formula (I) and L³ represents a suitable leaving group, such as a halogen atom (e.g. a chlorine atom). Use of a compound of formula (Xb) should be accompanied by a coupling agent.

Step (a) typically comprises reaction of a compound of formula (IX) with a compound of formula (Xa) when L³ is a leaving group such as halogen, in the presence of a suitable solvent, such as dichloromethane and a suitable base, such as diisopropylethylamine, triethylamine. Alternatively a compound of formula (IX) can be reacted with a compound of formula (Xb) in the presence of EDAC.HCl and optionally HOBt and optionally a suitable base.

Compounds of formula (IX) can be prepared in accordance with the following Scheme 3:

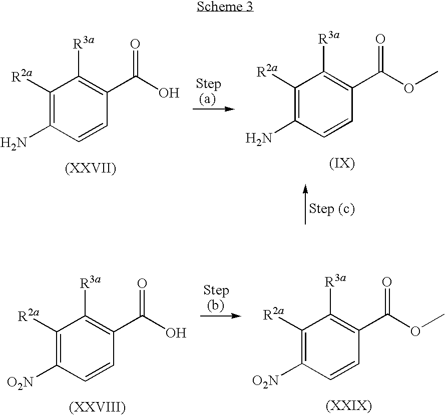

wherein R^{2a}, R^{3a} are R² or R³ as defined for compounds of formula (I) or a group which can be converted in a further reaction into a group R², R³ for example C₂₋₅alkenyl or NH₂. In the case of an alkenyl the conversion to an alkyl group may also occur in step (c).

Step (a) is an esterification reaction which can be carried out under standard conditions e.g. with methanol in the presence of an acid e.g. H₂SO₄.

Step (b) is an esterification reaction which can be carried out under standard conditions e.g. with a methanol in the presence of an acid e.g. H₂SO₄

Step (c) is a hydrogenation reaction which can be carried out under typical conditions.

Compounds of formula (VIII) may be prepared from compounds of formula (IV) wherein Ra represents hydrogen in accordance with the following Scheme 4;

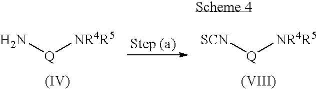

wherein Q, R⁴ and R⁵ are as defined above for compounds of formula (I).

Step (a) typically comprises the use of a suitable reagent, such as carbon disulfide, in the presence of a coupling agent such as dicyclohexylcarbodiimide and a suitable solvent, such as dry diethyl ether. Alternatively, step (a) may comprise the use of thiocarbonyldimidazole in a suitable solvent such as tetrahydrofuran or DMF.

Compounds of formula (III) wherein L¹ represents —SO₂-benzyl may be prepared in accordance with the following Scheme 5:

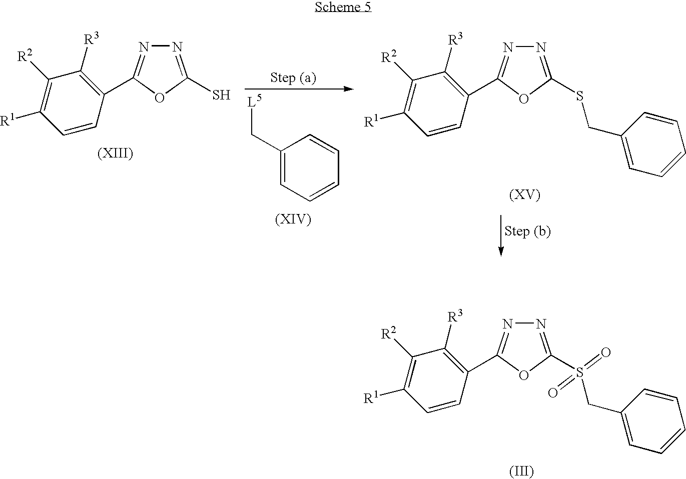

wherein R¹, R² and R³ are as defined above for compounds of formula (I) and L⁵ represents a suitable leaving group, such as a halogen atom (e.g. chlorine or bromine, more usually bromine).

Step (a) typically comprises reaction of a compound of formula (XIII) with a compound of formula (XIV) in the presence of a suitable solvent, such as ethanol and a suitable base, such as triethylamine.

Step (b) typically comprises the use of a suitable oxidising agent, such as meta-chloroperbenzoic acid (mCPBA) in the presence of a suitable solvent, such as dichloromethane or dichloromethane/THF mixture.

It will be appreciated that compounds of formula (III) wherein L¹ represents —SO₂—C₁₋₆ alkyl may be prepared in an analogous manner to Scheme 5, for example, the compound of formula (XIV) could be replaced with a compound of formula L⁵-C₁₋₆alkyl.

Compounds of formula (III) wherein L¹ represents halogen e.g. bromine may be prepared in accordance with the following Scheme 6.

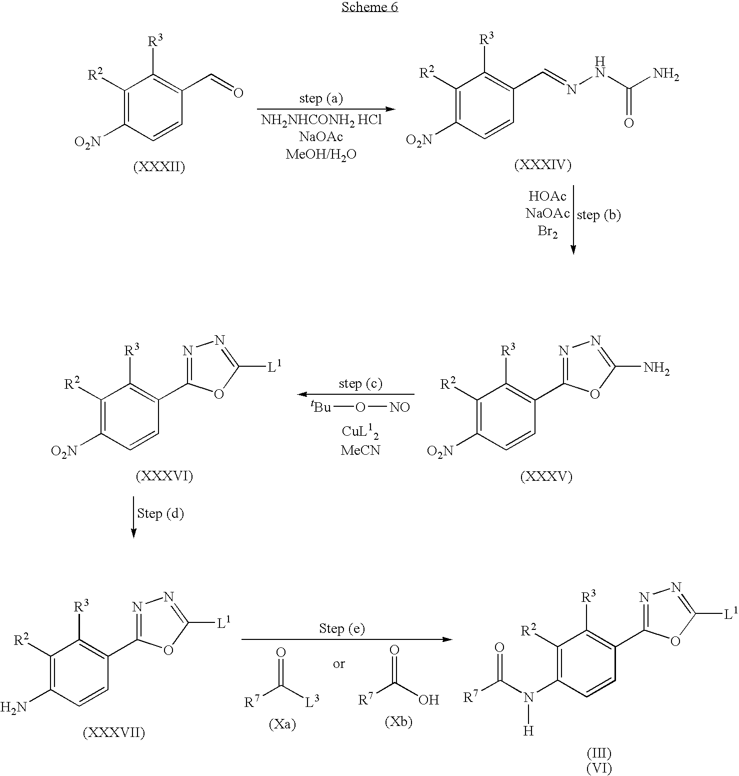

Step (a) typically comprises the use of NH₂NHCONH₂.HCl and sodium acetate in the presence of a suitable solvent such as aqueous methanol.

Step (b) typically comprises the use of bromine and sodium acetate in the presence of a suitable solvent such as acetic acid.

Step (c) typically comprises the use of t-butyl nitrite and for example when L¹ is Br, copper (II) bromide in the presence of a suitable solvent such as acetonitrile.

Step (d) is a hydrogenation reaction under typically conditions.

Step (e) typically comprises reaction of a compound of formula (XXXVII) with a compound of formula (Xa) when L³ is a leaving group such as halogen, in the presence of a suitable solvent, such as dichloromethane and a suitable base, such as diisopropylethylamine, triethylamine. Alternatively a compound of formula (IX) can be reacted with a compound of formula (Xb) in the presence of EDAC.HCl and optionally HOBt and optionally a suitable base.

Compounds of formula (XIII) may be prepared in accordance with the following Scheme 7:

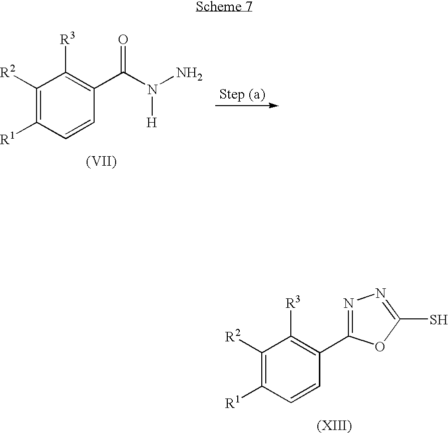

wherein R¹, R² and R³ are as defined above for compounds of formula (I).

Step (a) typically comprises the use of carbon disulfide and potassium hydroxide in the presence of a suitable solvent, such as ethanol.

Compounds of formula (V) may be prepared in accordance with the following Scheme 8. Compounds of formula (XVIII)^{p} are protected derivatives of compounds of formula (XVIII).

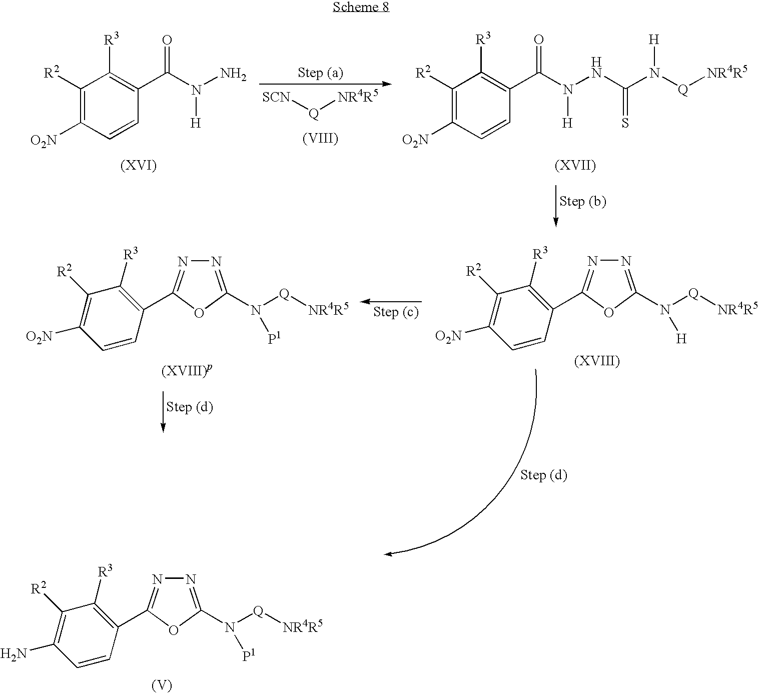

wherein Q, R², R³, R⁴ and R⁵ are as defined above for compounds of formula (I) and P¹ is hydrogen or a protecting group, for example Boc. It will be appreciated that compounds of formula (XVIII) can be protected as represented by compounds of formula (XVIII)^{p} wherein P¹ is a protecting group, for example a Boc protecting group. Compounds of formula (XVIII) may be used in step (d) as protected compounds of formula (XVIII)^{p} or unprotected compounds (XVIII).

Step (a) typically comprises the reaction of a compound of formula (XVI) with a compound of formula (VIII) in the presence of a suitable solvent, such as N,N-dimethylformamide or THF at a suitable temperature, e.g. room temperature.

Step (b) typically comprises a cyclisation reaction using a suitable reagent, such as N-cyclohexylcarbodiimide, N′-methyl polystyrene (P-DCC) or EDAC.HCl in the presence of a suitable solvent, such as N,N-dimethylformamide at a suitable temperature, e.g. 80° C. Alternatively KI/I₂ may be used.

Step (c) typically comprises protection of compound of formula (XVIII) with a suitable protecting group, such as Boc group under standard conditions.

Step (d) typically comprises a hydrogenation reaction according to standard procedures known in the art.

Compounds of formula (XVIII) may also be prepared by reaction of compounds of formula (XXXVI) wherein L¹ is Br with a compound of formula (IV) as shown in Scheme 9:

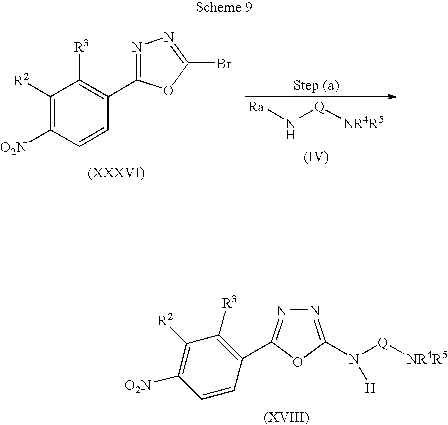

wherein Ra, Q, R², R³, R⁴ and R⁵ are as defined for compounds of formula (I).

Step a) typically uses a suitable solvent such as dioxane or IPA and a suitable base such as diisopropylethylamine, at a suitable temperature, such as room temperature to 80° C.

Compounds of formula (IV) wherein Ra is hydrogen may be prepared in accordance with the following Scheme 10:

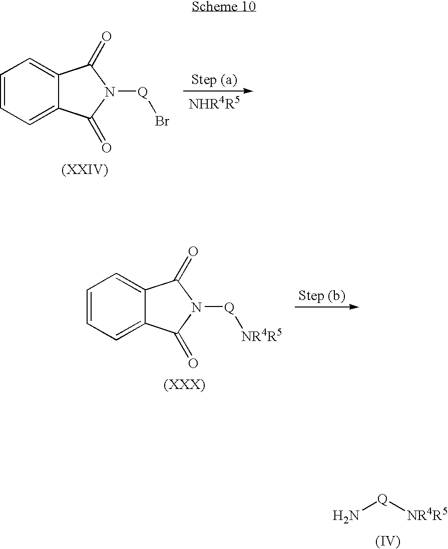

wherein Q, R⁴ or R⁵ are as described for compounds of formula (I).

Step a) is a reaction of NHR⁴R⁵ in the presence of a suitable base e.g. triethylamine in a suitable solvent such as ethanol at a suitable temperature, e.g. 80° C.

Step b) typically comprises the use of MeNH₂ in a suitable solvent such as ethanol at a suitable temperature such as room temperature or the use of NH₂NH₂.H₂O in a suitable solvent such as ethanol at a suitable temperature such as reflux.

Compounds of formula (IV) wherein Ra is hydrogen may also be prepared in accordance with the following Scheme 11:

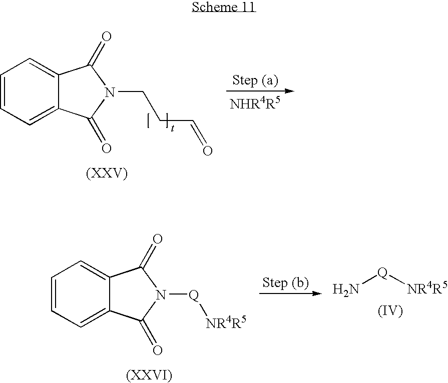

wherein R⁴, R⁵, and Q are as defined for compounds of formula (I) and t is 1 or 2.

Step a) is a reaction of NHR⁴R⁵ in the presence of a suitable reducing agent e.g. NaBH(OAc)₃ in a suitable solvent such as dichloromethane at a suitable temperature, e.g. room temperature.

Step b) typically comprises the use of MeNH₂ in a suitable solvent such as ethanol at a suitable temperature such as room temperature or the use of NH₂NH₂.H₂O in a suitable solvent such as ethanol at a suitable temperature such as reflux.

Compounds of formula (IV) wherein Ra is methyl can be prepared from compounds of formula (IV) wherein Ra is hydrogen as shown in Scheme 12.

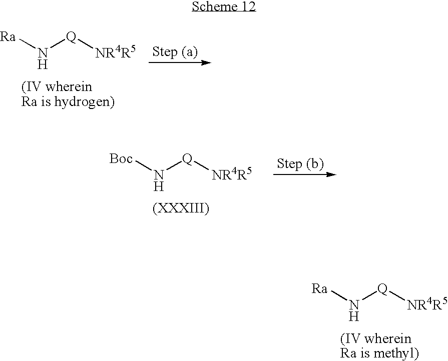

wherein Q, R⁴, R⁵ are as defined for compounds of formula (I).

Compounds of formula (IV) are protected in step (a) using standard reaction conditions to replace a hydrogen with a Boc group e.g. use of (Boc)₂O in dichloromethane.

Step (b) is a treatment with LiAlH₄.

The product of step (b) may be used in subsequent reactions without further purification.

Compounds of formula (I) wherein Ra is hydrogen, may also be prepared in accordance with the following Scheme 13 using solid phase synthesis:

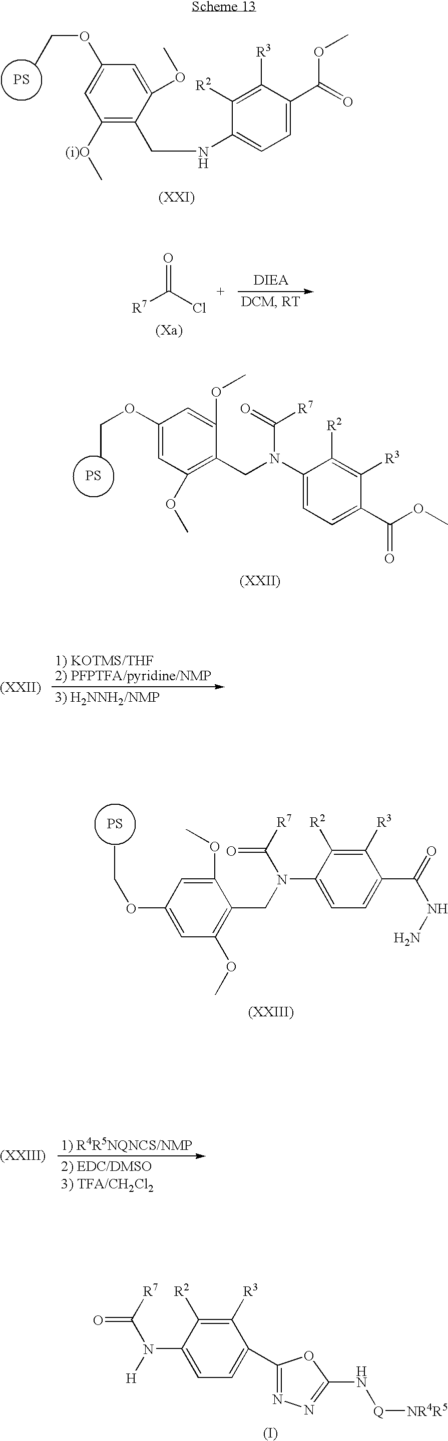

Resin-bound arylamino ester (XXI) is coupled with acid chloride (Xa) to form resin-bound benzamide (XXII). The resin-bound material is treated with a solution of potassium trimethylsilanoate (KOTMS) in tetrahydrofuran to saponify the ester, which is subsequently treated with pentafluorophenyl trifluoroacetate (PFPTFA) and pyridine in N-methyl pyrrolidinone (NMP) to produce the resin-bound pentafluorophenyl ester. This material is treated with a solution of hydrazine in NMP at room temperature to afford resin-bound hydrazide (XXIII). Treatment of (XXIII) with a solution of isothiocyanate (R⁴R⁵NQNCS) in NMP provides the resin-bound acyl thiosemicarbazide which can then be cyclized to the oxadiazole by treatment with N-(3-dimethylaminopropyl)-N′-ethylcarbodiimide hydrochloride (EDC) in dimethyl sulfoxide (DMSO) with heating. Cleavage of the resin-bound material using a solution of trifluoroacetic acid (TFA) in dichloromethane (CH₂Cl₂) affords desired product (I) in good yield and purity. Compounds may purified by reverse-phase HPLC as appropriate.

Compounds of formula (XXI) may be prepared by loading a solid support (XX) in accordance with the following Scheme 14:

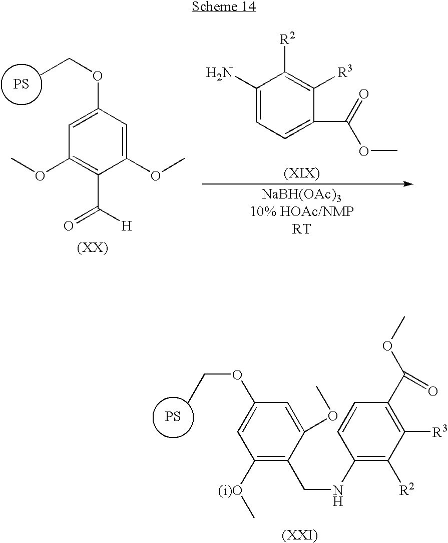

wherein PS indicates a polystyrene backbone of the support, and R² and R³ are as defined for compounds of formula (I). Arylamino esters (XIX) were loaded onto commercially available FDMP (formyldimethoxyphenoxy) resin (XX) to form resin-bound esters (XXI).

A further aspect of the invention is compounds of formula (B) and salts thereof: compounds of formula (I):

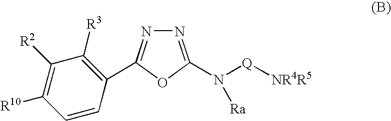

wherein
R¹⁰ represents —NO₂ and NH₂, and Q, Ra, R², R³, R⁴, and R⁵ are as defined for compounds of formula (I). Compound of formula (B) may have activity at the alpha 7 receptor and/or may be useful intermediates in the preparation of compounds of formula (I).

Compounds of formula (IV), (IX), (X), (XIV), (XV), (XVI), (XX), (XXIV), (XXV) and (XXXII) are either commercially available, or may be prepared by known methods.

Compounds of formula (I) and their pharmaceutically acceptable salts may have affinity for and be agonists at the nicotinic α7 receptor and are believed to be of potential use in the treatment of neurological diseases including Alzheimer's disease (particularly cognitive deficit of Alzheimer's disease), dementia (including Lewy body dementia and vascular dementia), age-related memory dysfunction, cognitive impairment as listed below, cognitive deficit especially cognative deficit of schizophrenia, Parkinson's disease and Tourette's syndrome, psychiatric disorders including schizophrenia as listed below, attention deficit/hyperactivity disorder as listed below, depression as listed below, anxiety as listed below and addiction, pain related disorders including pain of neuropathic origin including neuralgias, neuritis and back pain, and inflammatory pain including osteoarthritis, rheumatoid arthritis, acute inflammatory pain and back pain, migraine; and other diseases including obesity, sepsis and gastrointestinal disorders (including irritable bowel syndrome and inflammatory bowel disease). A further neurological disease for which these compounds may be of potential use is epilepsy.

The compounds of the formula may be useful in the treatment of pain.

When used herein the term pain, includes of neuropathic orignm including neuralgias, neuritis and back pain; acute pain, chronic pain, chronic articular pain, musculoskeletal pain, inflammatory pain including osteoarthritis, and rheumatoid arthritis, acute inflammatory pain and back pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

In one embodiment the compounds may be useful in the treatment of chronic pain, post-operative pain, chronic lower back and neck pain, cancer pain, sprains and strains.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

The following disease classification refer to the classification code in Diagnostic and Statistical Manual of Mental Disorders, 4th Edition, published by the American Psychiatric Association (DSM-IV) and/or the International Classification of Diseases, 10th Edition (ICD-10):

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).
ii) cognitive impairment including for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypothyroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.
iii) Depression and mood disorders for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90).
iv) Anxiety disorders for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00).
v) Attention-Deficit/Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-impulse Type (314.01) and Attention-Deficit/Hyperactivity Disorder Not Otherwise Specified (314.9)); Hyperkinetic Disorder; Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

When used herein the term treatment extends to as prophylaxis of the above disorders as well as treatment of established conditions.

Thus the invention also provides a compound of formula (I) or a pharmaceutically acceptable salt thereof, for use as a therapeutic substance in the treatment of the above disorders, in particular pain, neurological (e.g. cognitive deficit of Alzheimer's disease) and psychiatric disorders (e.g. cognitive deficit of schizophrenia).

The invention further provides a method of treatment of the above disorders, in mammals including humans, which comprises administering to the sufferer a therapeutically effective amount of a compound of formula (I) or a pharmaceutically acceptable salt thereof.

In another aspect, the invention provides the use of a compound of formula (I) or a pharmaceutically acceptable salt thereof in the manufacture of a medicament for use in the treatment of the above disorders.

When used in therapy, the compounds of formula (I) are usually formulated in a standard pharmaceutical composition. Such compositions can be prepared using standard procedures.

Thus, the present invention further provides a pharmaceutical composition for use in the treatment of the above disorders which comprises the compound of formula (I) or a pharmaceutically acceptable salt thereof and a pharmaceutically acceptable carrier.

The present invention further provides a pharmaceutical composition which comprises the compound of formula (I) or a pharmaceutically acceptable derivative thereof and a pharmaceutically acceptable carrier.

Compounds of formula (I) or a pharmaceutically acceptable derivative thereof may be used in combination with other therapeutic agents, for example medicaments indicated to be useful as either disease modifying or symptomatic treatments of Alzheimer's disease. Suitable examples of such other therapeutic agents may be agents known to modify cholinergic transmission such as 5-HT_{1A} antagonists, (e.g. lecozotan), 5-HT6 antagonists, M1 muscarinic agonists, M2 muscarinic antagonist, acetylcholinesterase inhibitors (e.g donepezil or rivastigmine), or allosteric modulators, nicotinic receptor agonists or allosteric modulators, symptomatic agents such as 5-HT6 receptor antagonists, H3 receptor antagonists, 5-HT4 receptor agonist, also NMDA receptor antagonists or modulators, and disease modifying agents such as β- or γ-secretase inhibitors (e.g. R-flurbiprofen). Other suitable examples of such other therapeutic agents may be medicaments indicated to be useful in the treatment of pain of neuropathic origin including neuralgias, neuritis and back pain, and inflammatory pain including osteoarthritis, rheumatoid arthritis, acute inflammatory pain, back pain and migraine.

The compounds of the invention may be used in combination with other therapeutic agents, for example COX-2 (cyclooxygenase-2) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; bisphosphonates, leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X_{4/7}. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable salt thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms may be prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

The composition may contain from 0.1% to 99% by weight, preferably from 10% to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three a day. Such therapy may extend for a number of weeks, months, years or even life.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### Description 1

### [4-(4-Morpholinyl)butyl]amine

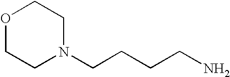

A mixture of 2-(4-bromobutyl)-1H-isoindole-1,3(2H)-dione (23.3 g, 82.6 mmol), morpholine (9.3 g, 106.95 mmol) and triethylamine (16.62 g, 164.5 mmol) in ethanol (250 mL) was heated under reflux for 6 hours, cooled, treated with hydrazine hydrate (9.5 g, 190 mmol) and heated under reflux for 2 hours then allowed to cool to room temperature overnight. The precipitated solid was filtered and the filtrate evaporated to dryness. The residue was dissolved in 2M hydrochloric acid (250 mL) and ethyl acetate (150 mL) and filtered to remove some insoluble material. The aqueous layer was separated, basified with 50% sodium hydroxide solution and extracted with dichloromethane (3×100 mL). The combined organics were dried over magnesium sulphate and evaporated to give the title compound (5.6 g) as a light brown oil.

### Description 2

### [4-(1-Piperidinyl)butyl]amine

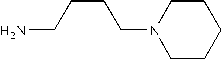

Piperidine (15.7 g, 185 mmol), 2-(4-bromobutyl)-1H-isoindole-1,3(2H)-dione (40.0 g. 142 mmol) and triethylamine (40 mL, 284 mmol) were heated to reflux in ethanol (400 mL) overnight. The reaction was allowed to cool to room temperature, hydrazine hydrate (16.4 g, 327 mmol) was then added and the reaction heated to reflux for 45 min. The solid formed was filtered off and washed with ethanol. The combined filtrate and washings were concentrated in vacuo and the residue triturated with diethyl ether (500 mL). The solid was filtered off and the filtrate concentrated in vacuo to give title compound (7.32 g) as a yellow oil.

The following amines in Table 1 may be prepared in a similar manner to that described in Descriptions 1 & 2 with variations in reactant equivalents, reaction times, work-up and purification methods as appropriate.

TABLE 1

| Compound Structure | Name |
|---|---|
| 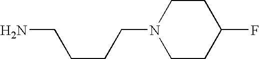 | [4-(4-Fluoro-1-piperidinyl)butyl]amine |
| 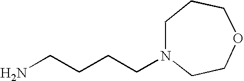 | [4-(Tetrahydro-1,4-oxazepin-4(5H)-yl)butyl]amine |

Alternatively, methylamine may be used instead of hydrazine hydrate for the deprotection, as illustrated by the following procedure

### Description 3

### [4-(4-Fluoro-1-piperidinyl)butyl]amine

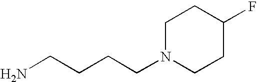

To a mixture of 4-fluoropiperidine hydrochloride (1.28 g, 9.2 mmol) and 2-(4-bromobutyl)-1H-isoindole-1,3(2H)-dione (7.1 mmol) in ethanol (25 mL) was added triethylamine (3.0 mL, 22 mmol) and the mixture heated to reflux for 5 h. The solid formed on cooling was filtered off and discarded. The solvent was removed in vacuo and the resulting solid was triturated with dichloromethane with a few drops of methanol. White solid was collected and the process repeated due to low recovery. To the combined solids (900 mg) was added methylamine in ethanol (33% by weight, 10 mL) and the solution stirred overnight. The reaction mixture was concentrated slightly in vacuo and diethyl ether (50 mL) added. The precipitate was filtered off and the filtrate concentrated in vacuo to give the title compound (355 mg).

### Description 4

### N-Cyclohexyl-N-methyl-1,3-propanediamine

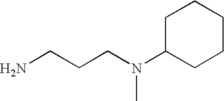

To a suspension of 3-(1,3-dioxo-1,3-dihydro-2H-isoindol-2-yl)propanal (200 mg, 0.98 mmol) in dichloromethane (5 mL) was added N-methylcyclohexanamine (88 mg, 0.78 mmol) in dichloromethane (5 mL) followed by sodium triacetoxyborohydride (311 mg, 1.47 mmol) and the reaction stirred at room temperature overnight. The reaction mixture was added to an SCX column, washed with methanol and eluted with 2M ammonia in methanol and the solvent removed in vacuo to give a yellow viscous oil (168 mg). An attempt to purify by SCX failed, so the mixture was separated between diethyl ether (10 mL) and aqueous sodium hydroxide (2M, 10 mL). The organic phase was concentrated in vacuo to give the title compound.

The following amines in Table 2 were made in a similar manner to that described in Description 4, with variations in reactant equivalents, reaction times and work up purifications as appropriate.

TABLE 2

| Compound Structure | Compound Name |
|---|---|
| 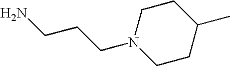 | 3-(4-methyl-1-piperidinyl)-1-propanamine |
| 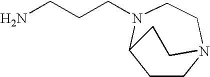 | 3-(1,4-Diazabicyclo[3.2.2]non-4-yl)-1-propanamine |
| 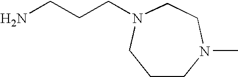 | 3-(4-Methylhexahydro-1H-1,4-diazepin-1-yl)-1-propanamine |
| 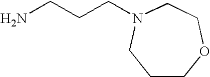 | 3-(Tetrahydro-1,4-oxazepin-4(5H)-yl)-1-propanamine |

Alternatively, hydrazine hydrate in refluxing ethanol may be used instead of methylamine for the deprotection as illustrated by the following Description.

### Description 5

### [3-(Tetrahydro-1,4-oxazepin-4(5H)-yl)propyl]amine

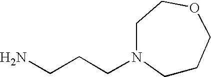

Hexahydro-1,4-oxazepine hydrochloride (1.0 g, 7.3 mmol) was partitioned between dichloromethane (15 mL) and aqueous sodium hydroxide (2M, 15 mL). The aqueous layer was further extracted with dichloromethane (2×10 mL). The combined organic layers were dried (phase separator) and to the solution was added 3-(1,3-dioxo-1,3-dihydro-2H-isoindol-2-yl)propanal (1.5 g, 7.3 mmol) and sodium triacetoxyborohydride (2.3 g, 11 mmol). The resulting solution was stirred at room temperature overnight. The reaction mixture was washed with aqueous sodium bicarbonate (45 mL) containing aqueous sodium hydroxide (2M 1 mL). The aqueous layer was extracted with further dichloromethane (40 mL) and the combined organic phase was dried (phase separator) and concentrated in vacuo to give an orange solid/gel (1.9 g, 6.5 mmol). The crude material was dissolved in ethanol (70 mL). Hydrazine hydrate (712 mg, 14.2 mmol) was added and the solution was heated to reflux for 2.5 h. The reaction was cooled to room temperature and allowed to stand overnight. The reaction mixture was partially concentrated in vacuo and diethyl ether (100 mL) was added. The resulting solid was filtered off and washed with further diethyl ether (25 mL). The filtrate was concentrated in vacuo and the product taken up in diethyl ether again (100 mL) and the solid filtered off. The filtrate was concentrated in vacuo to give the title compound (376 mg).

### Description 6

### Methyl[3-(1-piperidinyl)propyl]amine

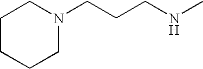

To a solution of [3-(1-piperidinyl)propyl]amine (1.0 g, 7.0 mmol) in dichloromethane (10 mL) was added di-tertbutyl dicarbonate (3.83 g, 17.6 mmol) and triethylamine (711 mg, 7.0 mmol) and the reaction stirred overnight. The solvent was removed in vacuo to give an orange oil. The oil was dissolved in tetrahydrofuran (5 mL) under argon at 0° C. and lithium aluminium hydride (1M solution in THF, 21 mL, 21 mmol) added slowly. The reaction was allowed to warm to room temperature, stirred for 1 h and left to stand overnight. The mixture was then cooled to 0° C. and aqueous sodium hydroxide (2M, 10 mL) was added. The solid formed was filtered off and the filtrate partitioned between water (30 mL) and dichloromethane (60 mL). The aqueous layer was extracted with further dichloromethane and the combined organic phases were isolated and dried (phase separator) and concentrated in vacuo to give a yellow oil (1.5 g). The title compound was present in this oil and the crude mixture was used without further purification.

### Description 7

### 1-(3-Isothiocyanatopropyl)piperidine

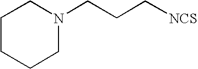

A solution of carbon disulfide (0.22 mL, 3.61 mmol) in dry diethyl ether (4 mL) was added dropwise to an ice cooled solution of 3-(1-piperidinyl)-1-propanamine (512 mg, 3.61 mmol) and dicyclohexylcarbodiimide (743 mg, 3.61 mmol) in dry diethyl ether (15 mL). The ice bath was removed after addition and the mixture stirred for 2 hours. The solid was filtered and the filtrate evaporated under reduced pressure to give the title compound. LC/MS (ES+ve): [M+H]⁺ at m/z 185 (C₉H₁₆N₂S requires [M+H]⁺ at m/z 185).

The following isothiocyanates in Table 3 may be prepared using the method described in Description 7 with variations in reactant equivalents, reaction times, work up or purifications as appropriate.

TABLE 3

| Structure | Name |
|---|---|
| 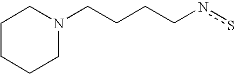 | 1-(4-Isothiocyanato-butyl)piperidine |
| 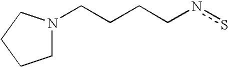 | 1-(4-Isothiocyanatobutyl)pyrroli-dine |

The following alternative procedure may be used for the preparation of isothiocyanates

### Description 8

### 1-(4-Isothiocyanatobutyl)piperidine

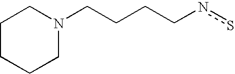

1,1-Thiocarbonyldiimidazole (178 mg, 1 mmol) was added at room temperature to a solution of 1-(aminobutyl)piperidine (229 mg, 1 mmol) in dimethylformamide (4 mL) and triethylamine (303 mg, 3 mmol). The mixture was stirred at room temperature for 18 hours and then diluted with water and extracted three times with ethyl acetate. The organic extracts were dried (sodium sulphate) and evaporated under reduced pressure to give the title compound (186 mg) as a yellow oil. LC/MS (ES+ve): [M+H]⁺ at m/z 199 (C₁₀H₁₈N₂S) requires [M+H]⁺ at m/z 199.

### Description 9

### Methyl 2-amino-4-nitrobenzoate

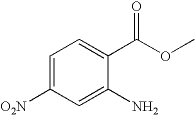

Concentrated sulphuric acid (4 mL) was added dropwise to a solution of 4-nitroanthranilic (2 g, 11 mmol) in methanol (40 mL) and the solution was heated under reflux overnight. The solvent was evaporated, the residue partitioned between ethyl acetate and saturated aqueous sodium bicarbonate solution. The organic layer was dried (magnesium sulphate), evaporated and triturated with ether to give the title compound (1.5 g) as a yellow solid.

### Description 10

### Methyl 2-bromo-4-nitrobenzoate

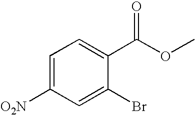

A mixture of copper (II) bromide (2.15 g, 9.6 mmol) and tert-butyl nitrite (1.6 mL, 13.2 mmol) in acetonitrile (30 mL) was heated to 65° C. and a solution of methyl 2-amino-4-nitrobenzoate (1.5 g, 7.7 mmol) in acetonitrile (10 mL) was added dropwise over 10 min. The mixture was heated at 65° C. for 1 hour, cooled, poured into 5M hydrochloric acid (100 mL) and extracted with ethyl acetate. The organic extract was dried (magnesium sulphate), evaporated and triturated with diethyl ether/hexane to give the title compound (1.33 g) as a yellow solid. LC/MS (ES+ve): [M+H]⁺ at m/z 260, 262 (C₈H₆BrNO₄ requires [M+H]⁺ at m/z 260,262).

### Description 11

### Methyl and Ethyl 2-ethenyl-4-nitrobenzoate

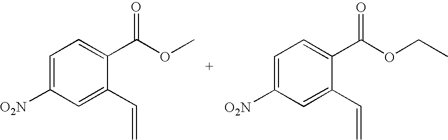

A mixture of methyl 2-bromo-4-nitrobenzoate (1.33 g, 5.1 mmol), vinylboronic anhydride pyridine complex (1.22 g, 5.1 mmol), tetrakis-(triphenylphosphine)palladium 0 (0.295 g, 0.255 mmol) and potassium carbonate (2.81 g, 20.4 mmol) in toluene:ethanol 1:1 (50 mL) was heated at 90° C. for 2 hours. The solvents were evaporated and the residue partitioned between ethyl acetate and water. The organic layer was dried (magnesium sulphate), evaporated and purified by column chromatography [silica gel, ethyl acetate:hexane (10%)] to give the title compound (0.98) as a 1:1 mixture of methyl and ethyl esters.

### Description 12

### Methyl 4-amino-2-ethylbenzoate

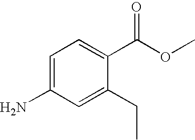

A solution of methyl and ethyl 2-ethenyl-4-nitrobenzoate (1:1 mixture of methyl and ethyl esters) 0.98 g assumed 4.7 mmol) in methanol (100 mL) was hydrogenated over 10% palladium on charcoal catalyst using an H-cube. The solution was evaporated to give the title compound (0.74 g) as 1:1 mixture of methyl and ethyl esters. LC/MS (ES+ve): [M+H]⁺ at m/z 180, 194 (C₁₀H₁₃NO₂ requires [M+H]⁺ at m/z 180), C₁₁H₁₅NO₂ requires [M+H]⁺ at m/z 194).

### Description 13

### Methyl 4-amino-3-iodobenzoate

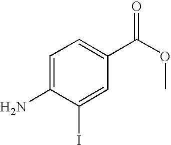

A suspension of methyl 4-aminobenzoate (4.53 g, 30 mmol) and N-iodosuccinimide (6.75 g, 30 mmol) in dimethylformamide (50 mL) was stirred at room temperature overnight. The reaction mixture was diluted with ethyl acetate, washed with water, 5% sodium thiosulphate solution and water, dried (magnesium sulphate) and evaporated. The residue was purified by flash chromatography [silica gel, ethyl acetate/hexane (0-25%)] to yield the title compound (8.2 g) as a yellow solid. LC/MS (ES+ve): [M+H]⁺ at m/z 278 (C₈H₈INO₂ requires [M+H]⁺ at m/z 278).

### Description 14

### Methyl 4-amino-3-ethenylbenzoate

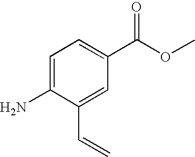

A mixture of methyl 4-amino-3-iodobenzoate (1.1 g, 4 mmol), vinylboronic anhydride pyridine complex (960 mg, 4 mmol), tetrakis-(triphenylphosphine)palladium 0 (0.231 g, 0.2 mmol) and potassium carbonate (2.2 g, 16 mmol) in toluene:ethanol 1:1 (40 mL) was heated at 90° C. for 1 hour. The reaction mixture was cooled and then portioned between ethyl acetate and water. The organic layer was dried (magnesium sulphate), evaporated and purified by column chromatography [silica gel, ethyl acetate/hexane (50%)] and triturated with hexane to give the title compound (0.54 g) as a white solid. LC/MS (ES+ve): [M+H]⁺ at m/z 178 (C₁₀H₁₁NO₂ requires [M+H]⁺ at m/z 178).

### Description 15

### Methyl 4-amino-3-ethylbenzoate

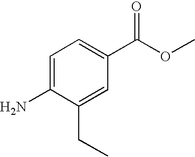

Methyl 4-amino-3-ethenylbenzoate (540 mg, 3.05 mmol) dissolved in ethanol (10 ml) and hydrogenated over 10% Pd/C catalyst (50 mg) overnight using an H-cube. The catalyst was filtered and the filtrate evaporated to give a mixture of starting material and required product. The solid was dissolved in methanol (30 ml) and hydrogenated over 10% Pd/C catalyst using the H-cube. The solvent was evaporated and the residue triturated with ether to yield the title compound (500 mg) as a white solid. LC/MS (ES+ve): [M+H]⁺ at m/z 180 (C₁₀H₁₃NO₂ requires [M+H]⁺ at m/z 180).

### Description 16

### Methyl 4-nitro-2-(trifluoromethyl)benzoate

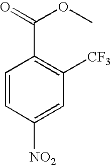

Sulphuric acid (4 mL) was slowly added to a solution of 4-nitro-2-(trifluoromethyl)benzoic acid in methanol (40 mL). The mixture was refluxed for 20 hours then evaporated to dryness. The residue dissolved in ethyl acetate and washed with saturated aqueous sodium bicarbonate solution, dried (magnesium sulphate) and evaporated. The residue was triturated with ether/hexane to give the title compound (2.2 g) as a white solid.

### Description 17

### Methyl 4-amino-2-(trifluoromethyl)benzoate

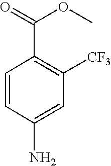

Methyl 4-nitro-2-(trifluoromethyl)benzoate (2 g, 8 mmol) was hydrogenated over palladium on carbon in methanol (160 mL) using an H-cube. The solution was evaporated to dryness to give the title compound (1.8 g) as a white solid.

LC/MS (ES+ve): [M+H]⁺ at m/z 220, (C₉H₈F₃NO₂ requires [M+H]⁺ at m/z 220

### Description 18

### Methyl 4-amino-2-fluorobenzoate

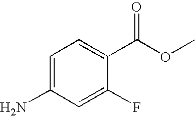

Concentrated sulphuric acid (12 mL) was added dropwise to a solution of 4-amino-2-fluorobenzoic acid (6 g, 35.5 mmol) in methanol (120 mL) and heated under reflux for 3 hours. The solvent was evaporated, the residue partitioned between ethyl acetate and water and basified with solid potassium carbonate. The layers were separated and the aqueous layer was extracted with ethyl acetate. The combined organics were dried (magnesium sulphate) and evaporated. The residue was triturated with ether to give the title compound (5.9 g) as a cream solid.

LC/MS (ES+ve): [M+H]⁺ at m/z 170 (C₈H₈FNO₂ requires [M+H]⁺ at m/z 170).

The following esters in Table 4 may be prepared by the method described above with variations in reactant equivalents, reaction times, work up or purifications as appropriate.

TABLE 4

| Structure | Name | [M + H]⁺ |
|---|---|---|
| 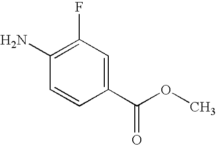 | Methyl 4-amino-3-fluorobenzoate | 170 |
| 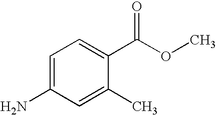 | Methyl 4-amino-2-methylbenzoate | 166 |

### Description 19

### Methyl 4-{[(2,6-difluorophenyl)carbonyl]amino}benzoate

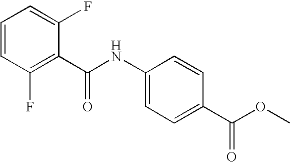

To a solution of methyl 4-aminobenzoate (1.51 g, 9.99 mmol) in dichloromethane (50 mL) was added triethylamine (1.53 mL, 10.99 mmol) followed by 2,6-difluorobenzoyl chloride (1.32 mL, 10.49 mmol). The reaction mixture was stirred at ambient temperature overnight. Saturated aqueous sodium bicarbonate solution (50 mL) and dichloromethane (200 mL) were then added and the organic layer separated. The organic solution was then washed with brine (50 mL), dried (Na₂SO₄) and concentrated. The resulting solid was recrystallised from industrial methylated spirit to give the title compound (2.3 g) as a white solid. LC/MS (ES+ve): [M+H]⁺ at m/z 292 (C₁₅H₁₁F₂NO₃ requires [M+H]⁺ at m/z 292).

The following compounds in Table 5 were prepared in a similar manner from the appropriate amine and acid chloride.

TABLE 5

| Structure | Name | [M + H]⁺ |
|---|---|---|
| 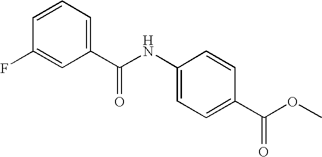 | Methyl 4-{[(3-fluorophenyl)carbonyl]amino}benzoate | 274 |
| 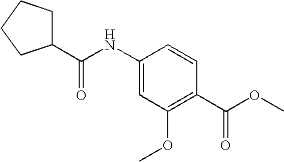 | Methyl 4-[(cyclopentylcarbonyl)amino]-2-(methyloxy)benzoate | 278 |
| 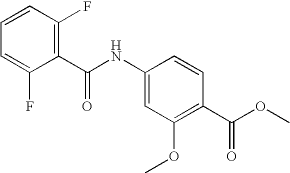 | Methyl 4-{[(2,6-difluorophenyl)carbonyl]amino}-2-(methyioxy)benzoate | 322 |
| 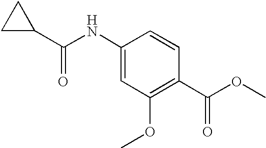 | Methyl 4-[(cyclopropylcarbonyl)amino]-2-(methyloxy)benzoate | 250 |
| 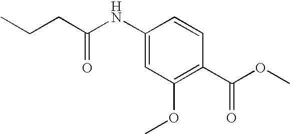 | Methyl 4-(butanoylamino)-2-(methyloxy)benzoate | 252 |
| 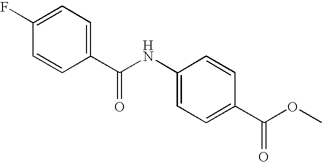 | Methyl 4-{[(4-fluorophenyl)carbonyl]amino}benzoate | 274 |
| 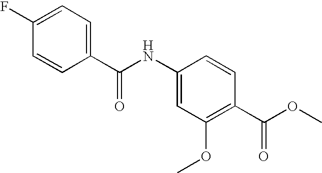 | Methyl 4-{[(4-fluorophenyl)carbonyl]amino}-2-(methyloxy)benzoate | 304 |
| 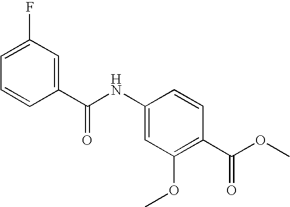 | Methyl 4-{[(3-fluorophenyl)carbonyl]amino}-2-(methyloxy)benzoate | 304 |

### Description 20

### Methyl 4-{[(2-fluorophenyl)carbonyl]amino}-2-(methyloxy)benzoate

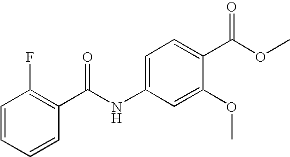

Methyl 4-amino-2-(methyloxy)benzoate (10.0 g, 55.2 mmol) was suspended in dichloromethane (50 mL), cooled in an ice bath and treated dropwise with diisopropylethylamine (10.57 mL, 60.8 mmol) followed by 2-fluorobenzoyl chloride (7.25 mL, 60.8 mmol). The mixture was allowed to warm to room temperature and stirred under argon for 2.5 hours. The solvent was evaporated under reduced pressure. The residue was taken up in ethyl acetate, washed with saturated aqueous sodium bicarbonate and brine. The ethyl acetate layer was separated. A solid came out of solution upon standing. This was collected by filtration to give the title compound. LC/MS (ES+ve): [M+H]⁺ at m/z 304 (C₁₆H₁₄FNO₄ requires [M+H]⁺ at m/z 304).

### Description 21

### Methyl 4-{[(2-fluorophenyl)carbonyl]amino}benzoate

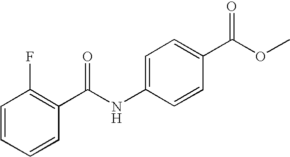

Methyl 4-aminobenzoate (3.83 g, 25.36 mmol) was suspended in dichloromethane (25 mL), cooled in an ice bath and treated dropwise with diisopropylethylamine (4.85 mL, 27.9 mmol) followed by 2-fluorobenzoyl chloride (3.33 mL, 27.9 mmol). The mixture was allowed to warm to room temperature and stirred under argon for 2 hours. The resulting solid was collected by filtration. The filtrate was allowed to stand and further solid came out of solution. This was collected by filtration, combined with the first batch of solid and dissolved in methanol. The solution was applied to an ion exchange cartridge (SCX) and washed with methanol. The product containing fractions were combined and evaporated to afford the title product. LC/MS (ES+ve): [M+H]⁺ at m/z 274 (C₁₅H₁₂FNO₃ requires [M+H]⁺ at m/z 274).

### Description 22

### Methyl 2-fluoro-4-{[(2-fluorophenyl)carbonyl]amino}benzoate

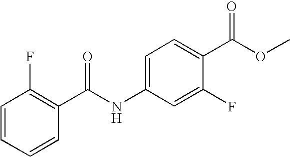

Methyl 4-amino-2-fluorobenzoate (3.83 g, 22.7 mmol) and triethylamine (3.81 mL, 27.4 mmol) were dissolved in dichloromethane (40 mL) and treated dropwise with 2-fluorobenzoyl chloride (3.25 mL, 27.4 mmol). The mixture was stirred for 1 hour at room temperature. The solution was washed with water, dried (magnesium sulphate) and evaporated. The resulting solid was triturated with ether to give the title compound (6.13 g). as a whitish solid. LC/MS (ES+ve): [M+H]⁺ at m/z 292 (C₁₅H₁₁F₂NO₃ requires [M+H]⁺ at m/z 292).

### Description 23

### (Methyl and Ethyl) 2-ethyl-4-{[(2-fluorophenyl)carbonyl]amino}benzoate

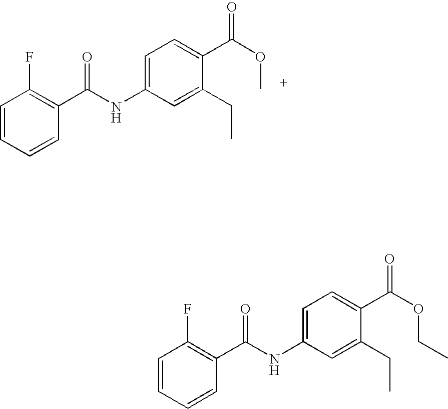

(Methyl and ethyl) 4-amino-2-ethylbenzoate (as 1:1 mixture of methyl and ethyl esters 0.735 g, assumed 4.1 mmol) and triethylamine (0.626 mL, 4.5 mmol) were dissolved in dichloromethane (8 mL), cooled in an ice bath and treated with 2-fluorobenzoyl chloride (0.54 mL, 4.5 mmol). The mixture was allowed to warm to room temperature and stirred for 2 hours. The solution was washed with water, dried (magnesium sulphate) and evaporated. The resulting solid was triturated with ether to give the title compound (1.2 g) as a 1:1 mixture of methyl and ethyl esters. LC/MS (ES+ve): [M+H]⁺ at m/z 302, 316 (C₁₇H₁₆FNO₃ requires [M+H]⁺ at m/z 302), (C₁₈H₁₈FNO₃ requires [M+H]⁺ at m/z 316).

The following compounds in Table 6 were prepared using a similar method to that described above using the appropriate aniline and acid chloride with variations in reactant equivalents, reaction times, work up or purifications as appropriate. The base may be either triethylamine or diisopropylethyl amine, the reaction temperature may be between 0° C. and ambient temperature, the number of equivalents of acid chloride may be varied, for example 1-1.2 equivalents may be used. The method of purification may vary and may involve aqueous work-up, trituration or column chromatography.

TABLE 6

| Structure | Name | [M + H]⁺ |
|---|---|---|
| 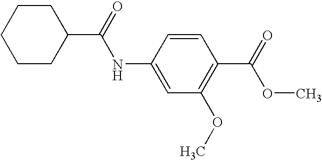 | Methyl 4-[(cyclohexylcarbonyl)amino]-2-(methyloxy)benzoate | 292 |
| 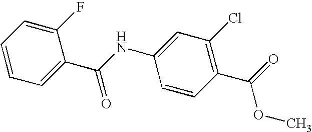 | Methyl 4-[(cyclohexylcarbonyl)amino]-2-(methyloxy)benzoate | 308 |
| 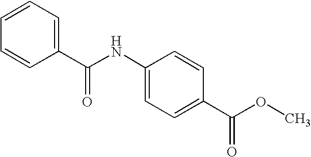 | Methyl 4-[(phenylcarbonyl)amino]benzoate | 256 |
| 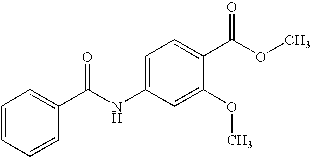 | Methyl 2-(methyloxy)-4-[(phenylcarbonyl)amino]benzoate | 286 |
| 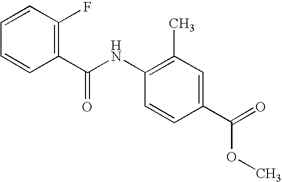 | Methyl 4-{[(2-fluorophenyl)carbonyl]amino}-3-methylbenzoate | 288 |
| 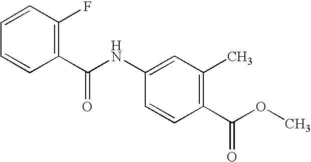 | Methyl 4-{[(2-fluorophenyl)carbonyl]amino}-2-methylbenzoate | 288 |
| 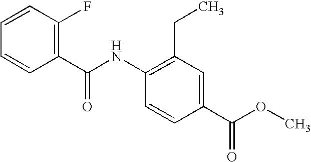 | Methyl 3-ethyl-4-{[(2-fluorophenyl)carbonyl]amino}benzoate | 302 |
| 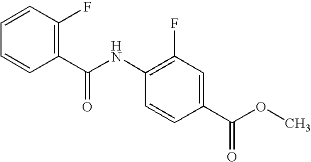 | Methyl 3-fluoro-4-{[(2-fluorophenyl)carbonyl]amino}benzoate | 292 |
| 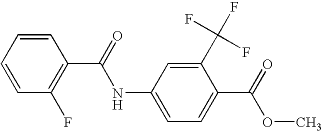 | Methyl 4-{[(2-fluorophenyl)carbonyl]amino}-2-(trifluoromethyl)benzoate | 342 |
| 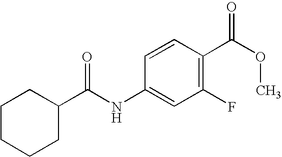 | Methyl 4-[(cyclohexylcarbonyl)amino]-2-fluorobenzoate | 280 |
| 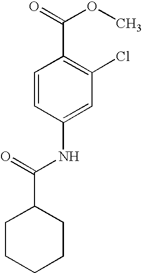 | Methyl 2-chloro-4-[(cyclohexylcarbonyl)amino]benzoate | 296 |
| 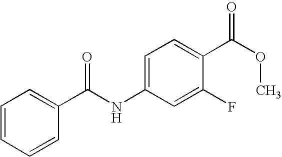 | methyl 2-fluoro-4-[(phenylcarbonyl)amino]benzoate | 274 |
| 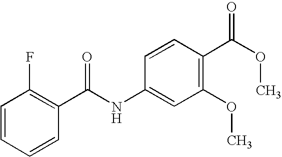 | Methyl 4-{[(2-fluorophenyl)carbonyl]amino}-2-(methyloxy)benzoate | 304 |
| 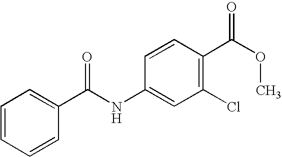 | Methyl 2-chloro-4-[(phenylcarbonyl)amino]benzoate | 290 |
| 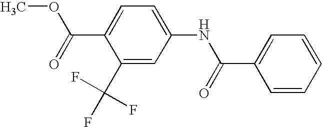 | Methyl 4-[(phenylcarbonyl)amino]-2-(trifluoromethyl)benzoate | 324 |
| 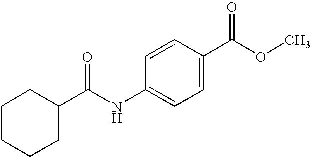 | Methyl 4-[(cyclohexylcarbonyl)amino]benzoate | 262 |
| 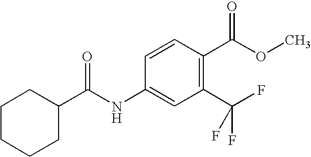 | Methyl 4-[(cyclohexylcarbonyl)amino]-2-(trifluoromethyl)benzoate | 330 |

### Description 24

### 2-Ethyl-4-{[(2-fluorophenyl)carbonyl]amino}benzoic acid

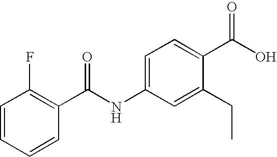

(Methyl and ethyl) 2-ethyl-4-{[(2-fluorophenyl)carbonyl]amino}benzoate (as a 1:1 mixture of methyl and ethyl esters (8.85 g, assumed 29.4 mmol) was dissolved in ethanol (150 mL) and treated with sodium hydroxide (2.35 g, 58.8 mmol) in water (30 mL). The mixture was stirred at room temperature then heated to 60° C. for 24 hours. The solvent was evaporated and the residue dissolved in water and washed with dichloromethane. The aqueous layer was acidified with 2M hydrochloric acid and the precipitated solid dissolved in ethyl acetate. The organic solution was dried, evaporated and triturated with ether to give the title compound (5.96 g) as a white solid. LC/MS (ES+ve): [M+H]⁺ at m/z (C₁₆H₁₄FNO₃ requires [M+H]⁺ at m/z 287).

### Description 25

### 1,1-Dimethylethyl 2-[(2-ethyl-4-{[(2-fluorophenyl)carbonyl]amino}phenyl)carbonyl]hydrazinecarboxylate

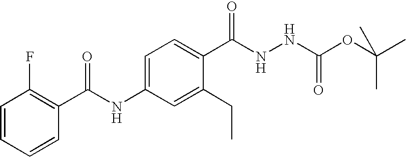

A solution of 2-ethyl-4-{[(2-fluorophenyl)carbonyl]amino}benzoic acid (5.49 g, 19.2 mmol), tert-butyl carbazate (3.04 g, 23 mmol), N-(3-dimethylaminopropyl)-N′-ethylcarbodiimide hydrochloride (4.03 g, 21.1 mmol) and 1-hydroxybenzotriazole hydrate (3.23 g, 21.1 mmol) in dichloromethane (60 mL) was stirred for 3 days. The solvent was evaporated and the residue dissolved in ethyl acetate and washed with saturated aqueous sodium bicarbonate solution (2×190 mL), water (200 mL), dried and evaporated to give the title compound (7.71 g) as a colourless oil LC/MS (ES+ve): [M+H]⁺ at m/z 402 (C₂₁H₂₄FN₃O₄ requires [M+H]⁺ at m/z 402).

### Description 26

### N-[3-Ethyl-4-(hydrazinocarbonyl)phenyl]-2-fluorobenzamide

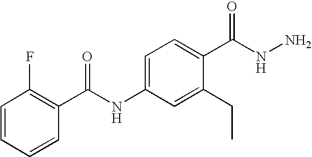

1,1-Dimethylethyl 2-[(2-ethyl-4-{[(2-fluorophenyl)carbonyl]amino}phenyl)carbonyl]hydrazinecarboxylate (7.7 g, 19.2 mmol) was stirred in 1,4-dioxan (50 mL) and treated with 4M hydrogen chloride in 1,4-dioxan (150 mL). The mixture was stirred for 2 days. The precipitated solid was filtered and dissolved in water. The solution was basified with 2M aqueous sodium hydroxide solution and extracted with dichloromethane. The organic solution was dried and evaporated to give the title compound (3.3 g). LC/MS (ES+ve): [M+H]⁺ at m/z 302 (C₁₆H₁₆FN₃O₂ requires [M+H]⁺ at m/z 302).

### Description 27

### 2,6-Difluoro-N-[4-(hydrazinocarbonyl)phenyl]benzamide

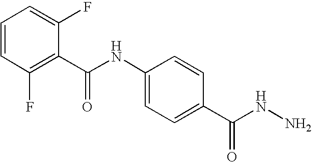

To a suspension of methyl 4-{[(2,6-difluorophenyl)carbonyl]amino}benzoate (2.3 g, 7.90 mmol) in methanol (40 mL) was added hydrazine monohydrate (3.1 mL, 63.18 mmol). The reaction mixture was heated at reflux over the weekend and then concentrated. The residue was triturated with industrial methylated spirit to give the title compound (0.91 g) as an off-white solid. LC/MS (ES+ve): [M+H]⁺ 292 at m/z (C₁₄H₁₁F₂N₃O₂ requires [M+H]⁺ at m/z 292).

The following compounds in Table 7 were prepared using a similar method to the above using the appropriate ester and hydrazine with variations in reactant equivalents, reaction times, work up or purifications as appropriate.

TABLE 7

| Structure | Name | LCMS [M + H]⁺ |
|---|---|---|
| 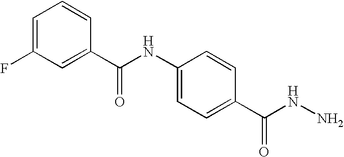 | 3-Fluoro-N-[4-(hydrazinocarbonyl)phenyl]benzamide | 274 |
| 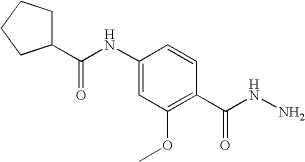 | N-{4-(Hydrazinocarbonyl)-3-(methyloxy)phenyl]cyclo-pentanecarboxamide | 278 |
| 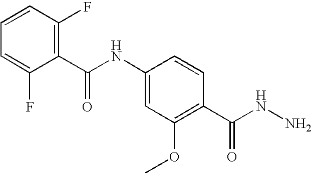 | 2,6-Difluoro-N-[4-(hydrazinocarbonyl)-3-(methyloxy)phenyl]benzamide | 322 |
| 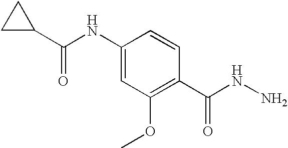 | N-[4-(Hydrazinocarbonyl)-3-(methyloxy)phenyl]cyclo-propanecarboxamide | 250 |
| 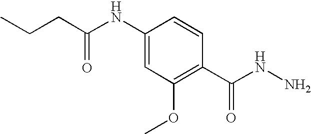 | N-[4-(Hydrazinocarbonyl)-3-(methyloxy)phenyl]butan-amide | 252 |
| 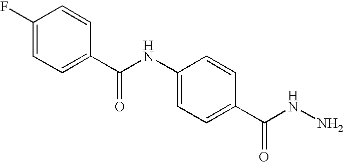 | 4-Fluoro-N-[4-(hydrazinocarbonyl)phenyl]benzamide | 274 |
| 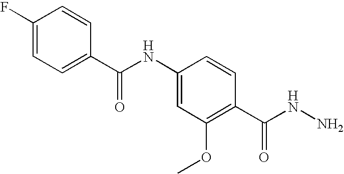 | 4-Fluoro-N-[4-(hydrazinocarbonyl)-3-(methyloxy)phenyl]benzamide | 304 |
| 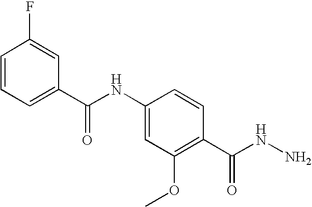 | 3-Fluoro-N-[4-(hydrazinocarbonyl)-3-(methyloxy)phenyl]benzamide | 304 |

### Description 28

### 2-Fluoro-N-[4-(hydrazinocarbonyl)-3-(methyloxy)phenyl]benzamide

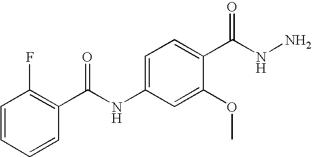

Methyl 4-{[(2-fluorophenyl)carbonyl]amino}-2-(methyloxy)benzoate (may be prepared as described in Description 20) (2.0 g, 6.60 mmol) was suspended in methanol (25 mL), treated with hydrazine monohydrate (1.60 mL, 33.0 mmol) dropwise and stirred at room temperature under argon for 1.5 hours. The mixture was heated under reflux, under argon for 18 hours. The mixture was cooled to room temperature, hydrazine monohydrate (0.80 mL, 16.5 mmol) was added and the mixture was heated under reflux under argon for 3.5 hours. The mixture was allowed to cool to room temperature and the resulting solid was filtered and washed with methanol. The solid was dried in a vacuum oven at 40° C. overnight to give the title compound. LC/MS (ES+ve): [M+H]⁺ at m/z 304 (C₁₅H₁₄FN₃O₃ requires [M+H]⁺ at m/z 304).

### Description 29

### 2-Fluoro-N-[4-(hydrazinocarbonyl)phenyl]benzamide

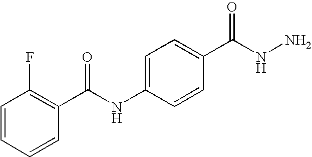

Methyl 4-{[(2-fluorophenyl)carbonyl]amino}benzoate (may be prepared as described in Description 21) (2.0 g, 7.33 mmol) was suspended in methanol (25 mL), treated with hydrazine monohydrate (2.66 mL, 54.98 mmol) dropwise and heated under reflux, under argon overnight. The mixture was allowed to cool to room temperature and the resulting solid was filtered and washed with methanol to give the title compound. LC/MS (ES+ve): [M+H]⁺ at m/z 274 (C₁₄H₁₂FN₃O₂ requires [M+H]⁺ at m/z 274).

### Description 30

### 2-Fluoro-N-[3-fluoro-4-(hydrazinocarbonyl)phenyl]benzamide

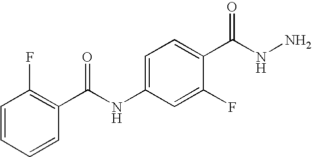

Methyl 2-fluoro-4-{[(2-fluorophenyl)carbonyl]amino}benzoate (6.37 g, 22 mmol) was suspended in methanol (65 mL), treated with hydrazine monohydrate (5.3 mL, 110 mmol) and heated under reflux, under argon for 18 hours. The mixture was allowed to cool to room temperature and the resulting solid was filtered, washed with methanol and ether. The solid was dried to give the title compound (5.8 g) as a white solid. LC/MS (ES+ve): [M+H]⁺ at m/z 292 (C₁₄H₁₁F₂N₃O₂) requires [M+H]⁺ at m/z 292.

The following compounds in Table 8 were prepared using a similar method to the above using the appropriate ester and hydrazine with variations in reactant equivalents, reaction times, work up or purifications as appropriate. The reaction time depends on the example and can be six or more days. In some cases further additions of hydrazine hydrate are required to give a good conversion of starting material to product. Sometimes the product can be isolated by filtration, alternatively, it may be purified by trituration, flash chromatography over silica gel or reversed phase chromatography.

TABLE 8

| Structure | Name | [M − H]⁺ |
|---|---|---|
| 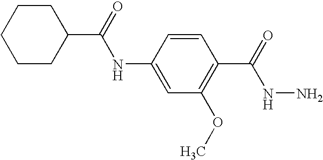 | N-[4-(hydrazinocarbonyl)-3-(methyloxy)phenyl]cyclohexanecarboxamide | 292 |
| 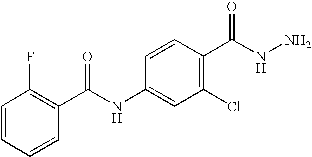 | N-[3-chloro-4-(hydrazinocarbonyl)phenyl]-2-fluorobenzamide | 308 |
| 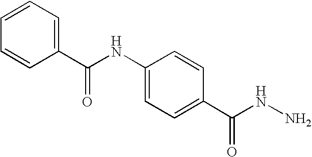 | N-[4-(hydrazinocarbonyl)phenyl]benzamide | 256 |
| 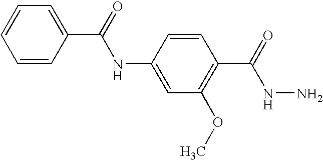 | N-[4-(hydrazinocarbonyl)-3-(methyloxy)phenyl]benzamide | 286 |
| 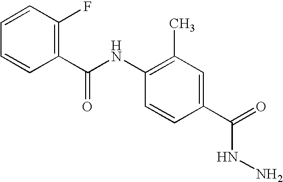 | 2-fluoro-N-[4-(hydrazinocarbonyl)-2-methylphenyl]benzamide | 288 |
| 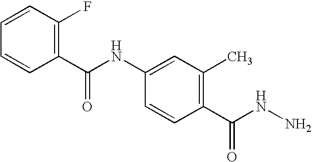 | 2-fluoro-N-[4-(hydrazinocarbonyl)-3-methylphenyl]benzamide | 288 |
| 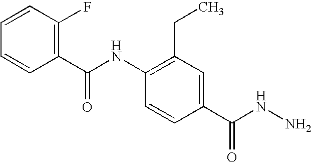 | N-[2-ethyl-4-(hydrazinocarbonyl)phenyl]-2-fluorobenzamide | 302 |
| 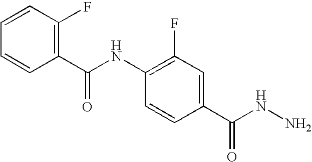 | 2-fluoro-N-[2-fluoro-4-(hydrazinocarbonyl)phenyl]benzamide | 292 |
| 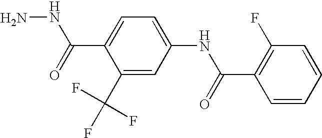 | 2-fluoro-N-[4-(hydrazinocarbonyl)-3-(trifluoromethyl)phenyl]benzamide | 342 |
| 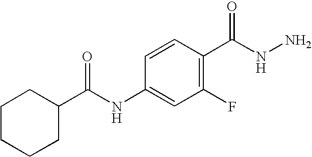 | N-[3-fluoro-4-(hydrazinocarbonyl)phenyl]cyclohexanecar-boxamide | 280 |
| 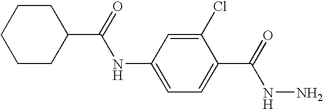 | N-[3-chloro-4-(hydrazinocarbonyl)phenyl]cyclohexanecar-boxamide | 296, 298 |
| 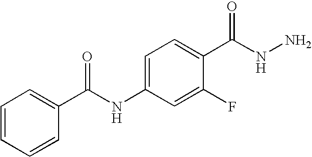 | N-[3-fluoro-4-(hydrazinocarbonyl)phenyl]benzamide | 274, 275 |
| 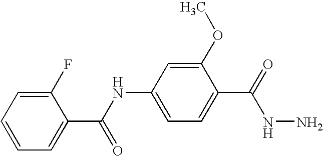 | 2-fluoro-N-[4-(hydrazinocarbonyl)-3-(methyloxy)phenyl]benzamide | 304 |
| 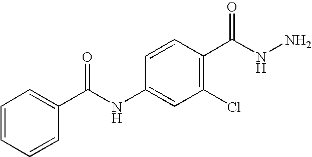 | N-[3-chloro-4-(hydrazinocarbonyl)phenyl]benzamide | [M − H]⁻288 |
| 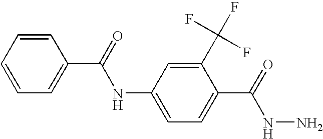 | N-[4-(hydrazinocarbonyl)-3-(trifluoromethyl)phenyl]benzamide | [M − H]⁻322 |
| 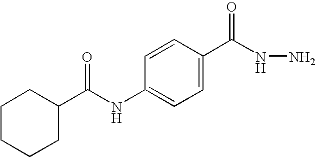 | N-[4-(hydrazinocarbonyl)phenyl]cyclohexanecar-boxamide | 262 |
| 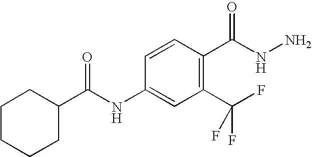 | N-[4-(hydrazinocarbonyl)-3-(trifluoromethyl)phenyl]cyclohexanecarboxamide | 330 |

### Description 31

### Ethyl 2-(ethyloxy)-4-nitrobenzoate

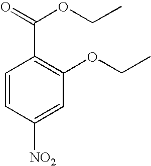

2-Hydroxy-4-nitrobenzoic acid (10.0 g, 54.6 mmol), iodoethane (17.0 g, 109 mol) and potassium carbonate (22.6 g, 164 mmol) were stirred in N,N-dimethylformamide (100 mL) at room temperature overnight. The reaction mixture was concentrated in vacuo and the residue partitioned between diethyl ether (200 mL) and aqueous sodium bicarbonate (200 mL). The organic phase was further washed with aqueous sodium bicarbonate (2×70 mL), isolated and the solvent removed in vacuo to give the title compound (10.35 g) as a yellow solid.

### Description 32

### 2-(Ethyloxy)-4-nitrobenzoic acid

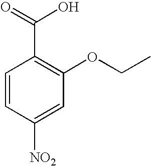

Ethyl 2-(ethyloxy)-4-nitrobenzoate (10.35 g) was dissolved in a mixture of NaOH (2M, 60 mL) and ethanol (180 mL) and stirred at room temperature. The reaction mixture was acidified, water added and the resultant solid filtered off and dried under vacuum to give the title compound (7.89 g). LCMS (ES-ve) [M−H]⁻ at m/z 210 (C₁₁H₁₃NO₅ requires [M−H]⁻ at m/z 210).

### Description 33

### 1,1-Dimethylethyl 2-{[2-(ethyloxy)-4-nitrophenyl]carbonyl}hydrazinecarboxylate

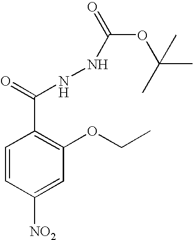

2-(Ethyloxy)-4-nitrobenzoic acid (7.89 g, 37.4 mmol), ^{t}butyl carbazate (4.94 g, 37.4 mmol) and N-[3-(dimethylamino)propyl]-N′-ethylcarbodiimide hydrochloride (8.60 g, 44.9 mmol) were stirred in dichloromethane (250 mL) at room temperature. Hydrochloric acid (2M, 70 mL) was added once reaction was complete and the layers separated. The organic phase was washed with further hydrochloric acid (2×70 mL) and it was then concentrated in vacuo to give the title compound (11.14 g) as a yellow solid. LCMS (ES-ve): [M−H]⁻ at m/z 324 (C₁₄H₁₉N₃O₆ requires [M−H]⁻ at m/z 324).

### Description 34

### 2-(Ethyloxy)-4-nitrobenzohydrazide

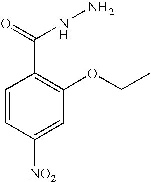

1,1-Dimethylethyl 2-{[2-(ethyloxy)-4-nitrophenyl]carbonyl}hydrazinecarboxylate (5.0 g, 15.4 mmol) was dissolved in 4M HCl in dioxane (125 mL) and the reaction mixture stirred for over 1 h. The reaction mixture was filtered and the solid washed with ether. The solid was then partitioned between dichloromethane (120 mL) and sodium bicarbonate (85 mL) plus aqueous sodium hydroxide (2M, 2 mL). The organic phase was then isolated (phase separator) and the solvent removed in vacuo to yield the title compound (2.82 g). LCMS (ES+ve): [M+H]⁺ at m/z 226 (C₉H₁₁N₃O₄ requires [M+H]⁺ at m/z 226).

### Description 35

### 2-Fluoro-N-(3-(methyloxy)-4-{[2-({[3-(1-piperidinyl)propyl]amino}carbonothioyl)hydrazino]carbonyl}phenyl)benzamide

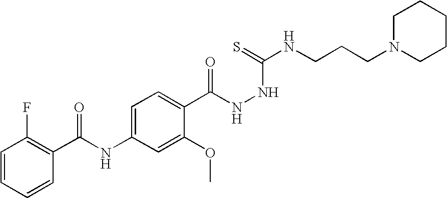

2-Fluoro-N-[4-(hydrazinocarbonyl)-3-(methyloxy)phenyl]benzamide (may be prepared as described in Description 28) (346 mg, 1.14 mmol) was suspended in N,N-dimethylformamide (5 mL), treated with 1-(3-isothiocyanatopropyl)piperidine (may be prepared as described in Description 7) (315 mg, 1.71 mmol) and stirred at room temperature under argon for 18 hours. The solvent was removed under reduced pressure and the resulting solid was triturated with dichloromethane. The solid was collected by filtration to give the title compound. LC/MS (ES+ve): [M+H]⁺ at m/z 488 (C₂₄H₃₀FN₅O₃S requires [M+H]⁺ at m/z 488).

### Description 36

### 2-Fluoro-N-(4-{[2-({[3-(1-piperidinyl)propyl]amino}carbonothioyl)hydrazino]carbonyl}phenyl)benzamide

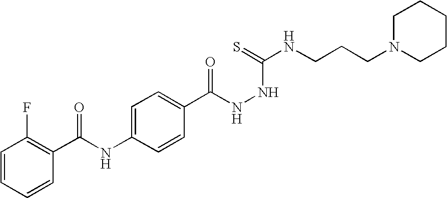

2-Fluoro-N-[4-(hydrazinocarbonyl)phenyl]benzamide (may be prepared as described in Description 29) (182 mg, 0.67 mmol) was suspended in N,N-dimethylformamide (3 mL), treated with 1-(3-isothiocyanatopropyl)piperidine (may be prepared as described in Description 7) 184 mg, 1.0 mmol) and stirred at room temperature under argon for 18 hours. The solvent was removed under reduced pressure and the resulting solid was triturated with diethyl ether. The solid was collected by filtration to give the title compound.

LC/MS (ES+ve): [M+H]⁺ at m/z 458 (C₂₃H₂₈FN₅O₂S requires [M+H]⁺ at m/z 458).

The following compounds in Table 9 were prepared in a similar manner from the appropriate hydrazide and isothiocyanate. In these three cases the products were purified by trituration with dichloromethane.

TABLE 9

| Structure | Name | [M + H]⁺ |
|---|---|---|
| 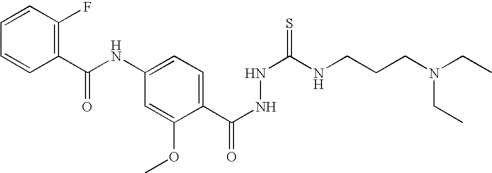 | N-[4-{[2-({[3-(diethylamino)propyl]amino}carbonothioyl)hydrazino]car-bonyl}-3-(methyloxy)phenyl]-2-fluorobenzamide | 476 |
| 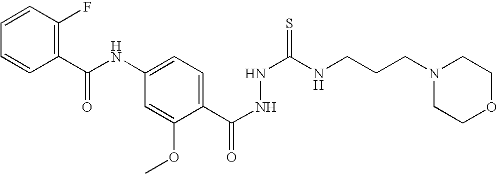 | 2-fluoro-N-(3-(methyloxy)-4-{[2-({[3-(4-morpholinyl)propyl]amino}carbonothioyl)hydrazino]car-bonyl}phenyl)benzamide | 490 |
| 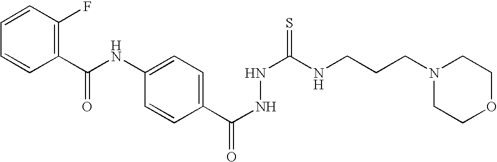 | 2-fluoro-N-(4-{[2-({[3-(4-morpholinyl)propyl]amino}carbonothioyl)hydrazino]car-bonyl}phenyl)benzamide | 460 |

### Description 37

### 2,6-Difluoro-N-(4-{[2-({[3-(1-piperidinyl)propyl]amino}carbonothioyl)hydrazino]carbonyl}phenyl)benzamide

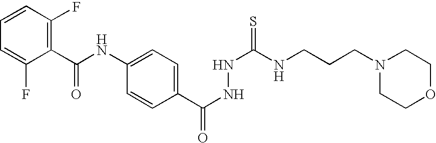

To a solution of 2,6-difluoro-N-[4-(hydrazinocarbonyl)phenyl]benzamide (600 mg, 2.06 mmol) in dimethylformamide (6 mL) was added 3-(4-morpholino)propyl isothiocyanate (0.34 mL, 1.98 mmol). The reaction mixture was heated at 30° C. overnight when LC/MS analysis showed 99% material with a retention time of 1.76 minutes. The reaction mixture was evaporated to leave a brown oil which was stirred overnight with dichloromethane. The resulting solid was filtered and dried to give the title compound (913 mg). LC/MS (ES+ve): [M+H]⁺ at m/z 478 (C₂₂H₂₅F₂N₅O₃S requires [M+H]⁺ at m/z 458).

The following compounds in Table 10 were prepared in a similar manner from the appropriate hydrazide and isothiocyanate with variations in reactant equivalents, reaction times, work up or purifications as appropriate:

TABLE 10

| Structure | Name | [M + H]⁺ |
|---|---|---|
| 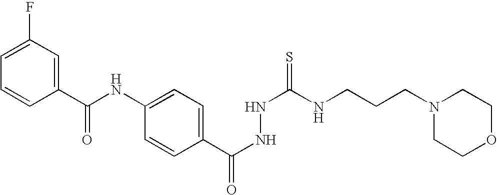 | 3-fluoro-N-(4-{[2-({[3-(4-morpholinyl)propyl]amino}car-bonothioyl)hydrazino]carbonyl}phenyl)benzamide | 460 |
| 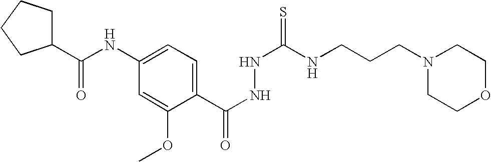 | N-(3-(methyloxy)-4-{[2-({[3-(4-morpholinyl)propyl]amino}car-bonothioyl)hydrazino]carbonyl}phenyl)cyclopentanecar-boxamide | 464 |
| 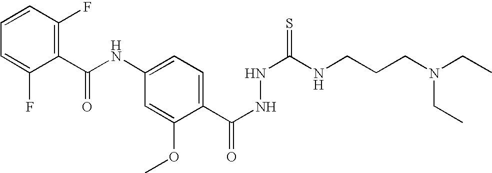 | N-[4-{[2-({[3-diethylamino)propyl]amino}carbonothioyl)hydrazino]car-bonyl}-3-(methyloxy)phenyl]-2,6-difluorobenzamide | 494 |
| 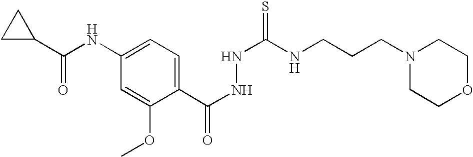 | N-(3-(methyloxy)-4-{[2-({[3-(4-morpholinyl)propyl]amino}car-bonothioyl)hydrazino]carbonyl}phenyl)cyclopropanecar-boxamide | 436 |
| 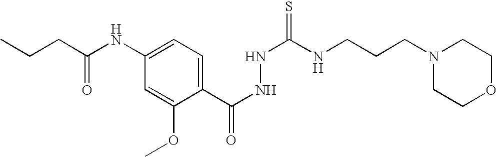 | N-(3-(methyloxy)-4-{[2-({[3-(4-morpholinyl)propyl]amino}car-bonothioyl)hydrazino]carbonyl}phenyl)butanamide | 438 |
| 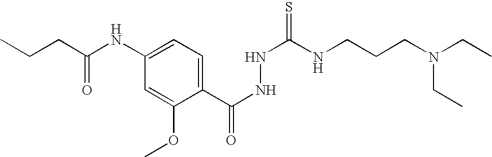 | N-[4-{[2-({[3-(diethylamino)propyl]amino}car-bonothioyl)hydrazino]car-bonyl}-3-(methyloxy)phenyl]butanamide | 424 |
| 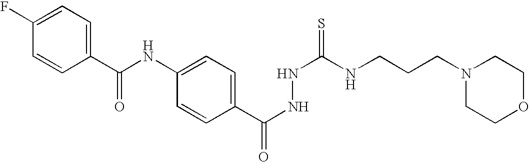 | 4-fluoro-N-(4-{[2-({[3-(4-morpholinyl)propyl]amino}car-bonothioyl)hydrazino]carbonyl}phenyl)benzamide | 460 |
| 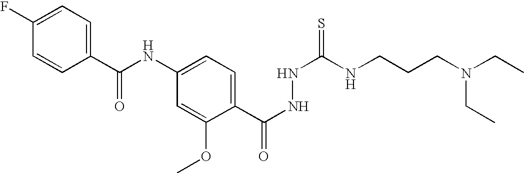 | N-[4-{[2-({[3-(diethylamino)propyl]amino}car-bonothioyl)hydrazino]carbonyl}-3-(methyloxy)phenyl]-4-fluorobenzamide | 476 |
| 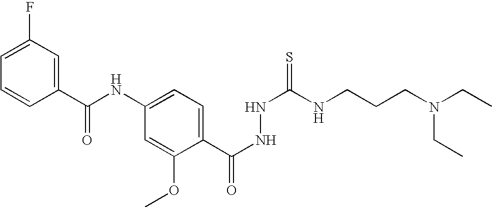 | N-[4-{[2-({[3-(diethylamino)propyl]amino}car-bonothioyl)hydrazino]carbonyl}-3-(methyloxy)phenyl]-3-fluorobenzamide | 476 |
| 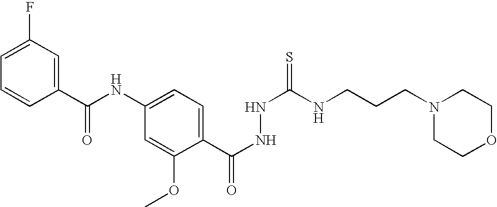 | 3-fluoro-N-(3-(methyloxy)-4-{[2-({[3-(4-morpholinyl)propyl]amino}car-bonothioyl)hydrazino]carbonyl}phenyl)benzamide | 490 |
| 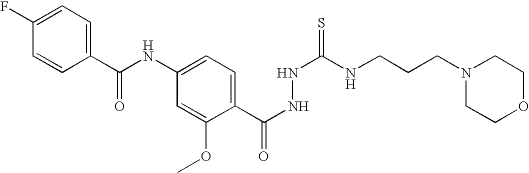 | 4-fluoro-N-(3-(methyloxy)-4-{[2-({[3-(4-morpholinyl)propyl]amino}car-bonothioyl)hydrazino]carbonyl}phenyl)benzamide | 490 |

### Description 38

### 2-Fluoro-N-(3-fluoro-4-{[2-({[3-(1-piperidinyl)propyl]amino}carbonothioyl)hydrazino]carbonyl}phenyl)benzamide

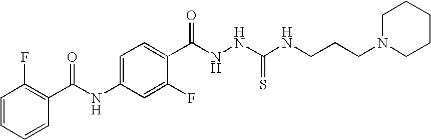

A suspension of 2-fluoro-N-[3-fluoro-4-(hydrazinocarbonyl)phenyl]benzamide (2.91 g, 10 mmol) and 4-(4-isothiocyanatopropyl)piperidine in tetrahydrofuran (30 mL) was heated at 60° C. for 3 hours. The yellow solution which formed was allowed to stand overnight at room temperature. The solvent was then evaporated and the residue triturated with ether and dried to yield the title compound (4.2 g) as a cream solid. This solid was used without further purification. LC/MS (ES+ve): [M+H]⁺ at m/z 476 (C₂₃H₂₇F₂N₅O₂S) requires [M+H]⁺ at m/z 476).

The following compounds in Table 11 may be prepared in a similar manner using the appropriate isothiocyanate and hydrazide with variations in reactant equivalents, reaction times, work up or purifications as appropriate. The reactions may be performed in either tetrahydrofuran or N,N-dimethylformamide as indicated.

TABLE 11

| | | Reaction | |
|---|---|---|---|
| Structure | Name | solvent | [M + H]⁺ |
| 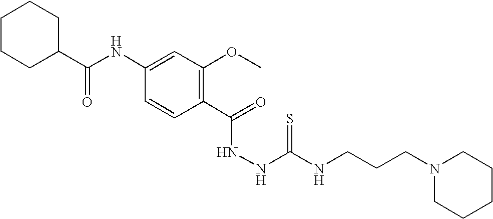 | N-(3-(Methyloxy)-4-{[2-({[3-(1-piperidinyl)propyl]amino}carbonothioyl)hydrazino]carbonyl}phenyl)cyclohexanecarboxamide | THF | 476 |
| 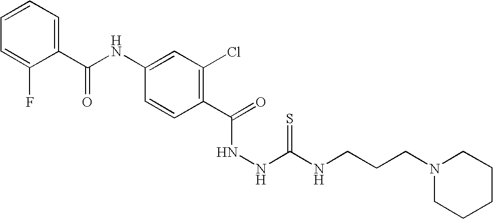 | N-(3-Chloro-4-{[2-({[3-(1-piperidinyl)propyl]amino}carbono-thioyl)hydrazino]carbonyl}phenyl)-2-fluorobenzamide | THF | 492 |
| 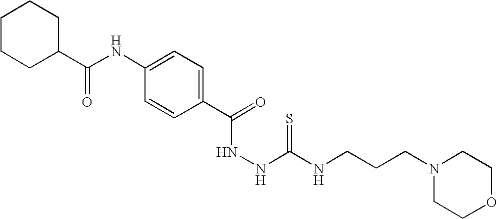 | N-(4-{[2-({[3-(4-Morpholinyl)propyl]amino}carbono-thioyl)hydrazino]carbonyl}phenyl)cyclohexanecarboxamide | THF | 448 |
| 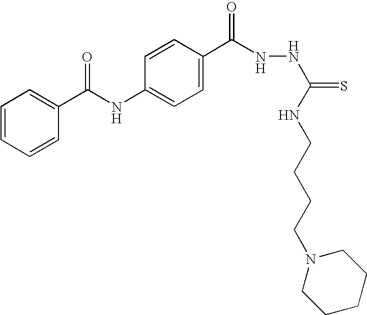 | N-(4-{[2-({[4-(1-Piperidinyl)butyl]amino}carbono-thioyl)hydrazino]carbonyl}phenyl)benzamide | THF | 454 |
| 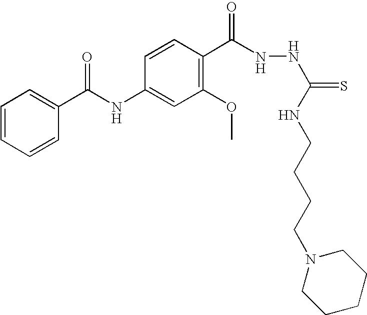 | N-(3-(Methyloxy)-4-{[2-({[4-(1-piperidinyl)butyl]amino}carbono-thioyl)hydrazino]carbonyl}phenyl)benzamide | THF | 484 |
| 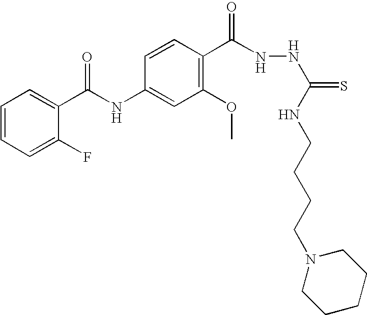 | 2-Fluoro-N-(3-(methyloxy)-4-{[2-({[4-(1-piperidinyl)butyl]amino}carbono-thioyl)hydrazino]carbonyl}phenyl)benzamide | THF | 502 |
| 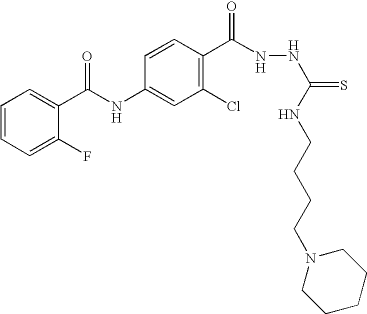 | N-(3-Chloro-4-{[2-({[4-(1-piperidinyl)butyl]amino}carbono-thioyl)hydrazino]carbonyl}phenyl)-2-fluorobenzamide | THF | 506 |
| 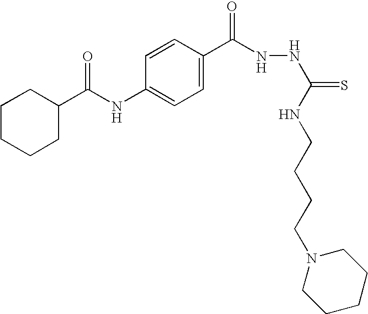 | N-(4-{[2-({[4-(1-piperidinyl)butyl]amino}carbono-thioyl)hydrazino]carbonyl}phenyl)cyclohexanecarboxamide | THF | 460 |
| 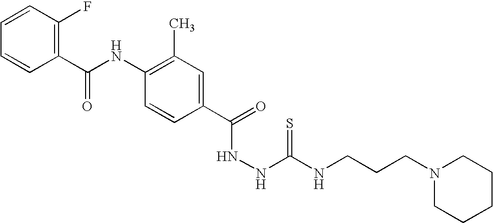 | 2-Fluoro-N-(2-methyl-4-{[2-({[3-(1-piperidinyl)propyl]amino}carbono-thioyl)hydrazino]carbonyl}phenyl)benzamide | DMF | 472 |
| 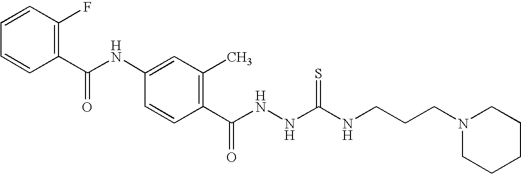 | 2-Fluoro-N-(3-methyl-4-{[2-({[3-(1-piperidinyl)propyl]amino}carbono-thioyl)hydrazino]carbonyl}phenyl)benzamide | THF | 472 |
| 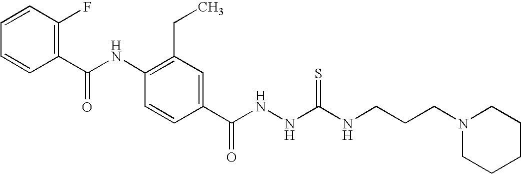 | N-(2-Ethyl-4-{[2-({[3-(1-piperidinyl)propyl]amino}carbono-thioyl)hydrazino]carbonyl}phenyl)-2-fluorobenzamide | THF | 486 |
| 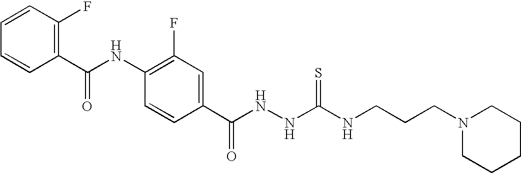 | 2-Fluoro-N-(2-fluoro-4-{[2-({[3-(1-piperidinyl)propyl]amino}carbono-thioyl)hydrazino]carbonyl}phenyl)benzamide | THF | 476 |
| 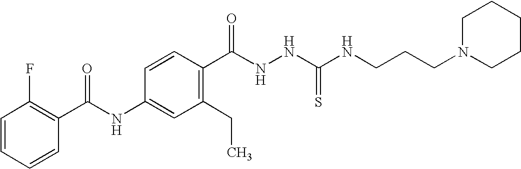 | N-(3-Ethyl-4-{[2-({[3-(1-piperidinyl)propyl]amino}carbono-thioyl)hydrazino]carbonyl}phenyl)-2-fluorobenzamide | THF | 486 |
| 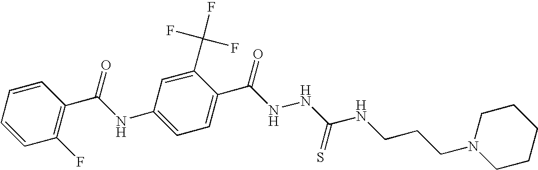 | 2-Fluoro-N-[4-{[2-({[3-(1-piperidinyl)propyl]amino}carbono-thioyl)hydrazino]carbonyl}-3-(trifluoromethyl)phenyl]benzamide | THF | 526 |
| 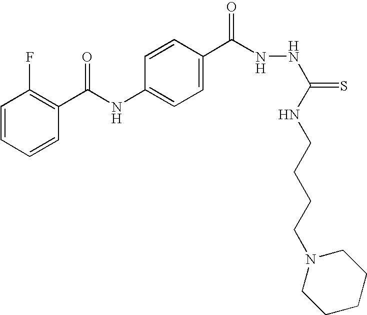 | 2-Fluoro-N-(4-{[2-({[4-(1-piperidinyl)butyl]amino}carbono-thioyl)hydrazino]carbonyl}phenyl)benzamide | THF | 472 |
| 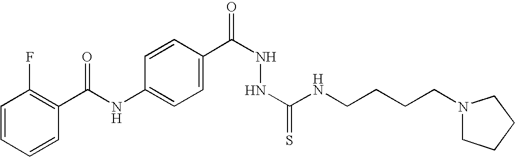 | 2-Fluoro-N-(4-{[2-({[4-(1-pyrrolidinyl)butyl]amino}carbono-thioyl)hydrazino]carbonyl}phenyl)benzamide | THF | 458 |

### Description 39

### 2-Fluoro-N-[4-(5-thioxo-4,5-dihydro-1,3,4-oxadiazol-2-yl)phenyl]benzamide

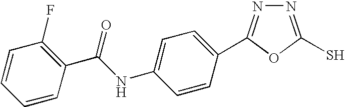

A mixture of 2-fluoro-N-[4-(hydrazinocarbonyl)phenyl]benzamide (may be prepared as described in Description 29) (5.0 g, 18.19 mmol) and potassium hydroxide (2.05 g 36.58 mmol) in ethanol (78 mL) was stirred at 0° C. under an atmosphere of argon. Carbon disulfide (3.3 mL, 54.87 mmol) was then added and the mixture stirred at 0° C. for a further 10 min and then allowed to warm to room temperature. After stirring at room temperature for 30 min the mixture was heated at 90° C. (block temperature) overnight. The mixture was then allowed to cool to room temperature and acidified with 2M hydrochloric acid. The title compound was obtained as a cream solid after collecting the resulting precipitate, washing with water and drying at 40° C. overnight under high vacuum.

LC/MS (ES+ve): [M+H]⁺ at m/z 316 (C₁₅H₁₀FN₃O₂S requires [M+H]⁺ at m/z 316).

### Description 40

### 2-Fluoro-N-[3-fluoro-4-(5-thioxo-4,5-dihydro-1,3,4-oxadiazol-2-yl)phenyl]benzamide

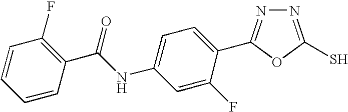

A mixture of 2-fluoro-N-[3-fluoro-4-(hydrazinocarbonyl)phenyl]benzamide (1.31 g, 4.5 mmol) and potassium hydroxide (0.504 g, 9 mmol) in ethanol (20 mL) was stirred under argon, treated with carbon disulfide (0.81 mL, 13.5 mmol) and heated under reflux for 5 days. The solvent was evaporated and the residue triturated with 2M hydrochloric acid. The solid was filtered washed with water and dried to give the title compound (1.3 g) as an off-white solid.

LC/MS (ES+ve): [M+H]⁺ at m/z 334 (C₁₅H₉F₂N₃O₂S requires [M+H]⁺ at m/z 334).

The following compounds in Table 12 may be prepared in a similar manner using the appropriate hydrazide with variations in reactant equivalents, reaction times, work up or purifications as appropriate. The reaction time may vary from less than a day to greater than 6 days. Purification may be by filtration or trituration.

TABLE 12

| Structure | Name | [M + H]⁺ |
|---|---|---|
| 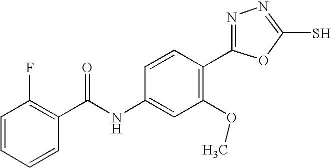 | 2-fluoro-N-[3-(methyloxy)-4-(5-thioxo-4,5-dihydro-1,3,4-oxadiazol-2-yl)phenyl]benzamide | 346 |
| 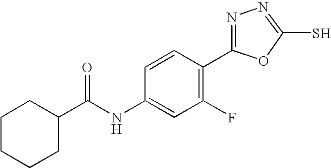 | N-[3-fluoro-4-(5-thioxo-4,5-dihydro-1,3,4-oxadiazol-2-yl)phenyl]cyclohexanecarboxamide | 322 |
| 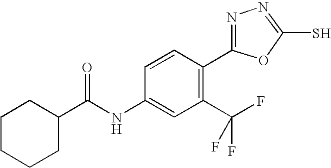 | N-[4-(5-thioxo-4,5-dihydro-1,3,4-oxadiazol-2-yl)-3-(trifluoromethyl)phenyl]cyclo-hexanecarboxamide | 372 |
| 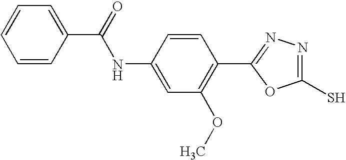 | N-[3-(methyloxy)-4-(5-thioxo-4,5-dihydro-1,3,4-oxadiazol-2-yl)phenyl]benzamide | 328 |
| 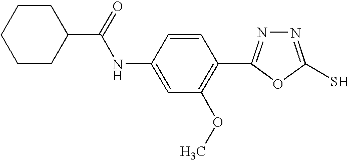 | N-[3-(methyloxy)-4-(5-thioxo-4,5-dihydro-1,3,4-oxadiazol-2-yl)phenyl]cyclohexanecarboxamide | 334 |
| 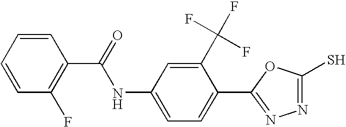 | 2-fluoro-N-[4-(5-thioxo-4,5-dihydro-1,3,4-oxadiazol-2-yl)-3-(trifluoromethyl)phenyl]benzamide | 384 |
| 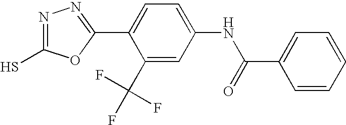 | N-[4-(5-thioxo-4,5-dihydro-1,3,4-oxadiazol-2-yl)-3-(trifluoromethyl)phenyl]benzamide | 366 |
| 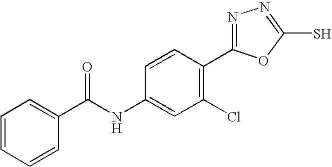 | N-[3-chloro-4-(5-thioxo-4,5-dihydro-1,3,4-oxadiazol-2-yl)phenyl]benzamide | 332, 334 |
| 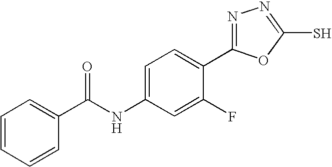 | N-[3-fluoro-4-(5-thioxo-4,5-dihydro-1,3,4-oxadiazol-2-yl)phenyl]benzamide | 316 |
| 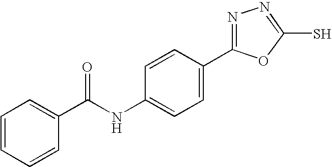 | N-[4-(5-thioxo-4,5-dihydro-1,3,4-oxadiazol-2-yl)phenyl]cyclohexanecarboxamide | 304 |
| 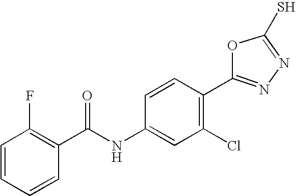 | N-[3-chloro-4-(5-thioxo-4,5-dihydro-1,3,4-oxadiazol-2-yl)phenyl]-2-fluorobenzamide | 350 |
| 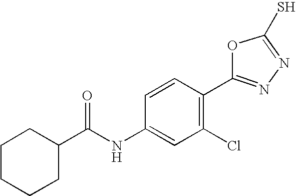 | N-[3-chloro-4-(5-thioxo-4,5-dihydro-1,3,4-oxadiazol-2-yl)phenyl]cyclohexanecarboxamide | 338 |
| 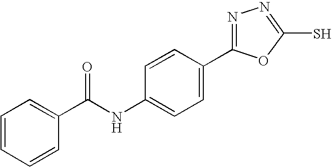 | N-[4-(5-thioxo-4,5-dihydro-1,3,4-oxadiazol-2-yl)phenyl]benzamide | 298 |
| 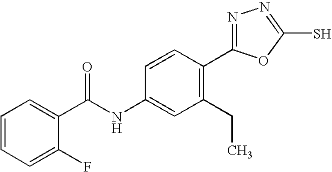 | N-[3-ethyl-4-(5-thioxo-4,5-dihydro-1,3,4-oxadiazol-2-yl)phenyl]-2-fluorobenzamide | 344 |

### Description 41

### 2-Fluoro-N-(4-{5-[(phenylmethyl)thio]-1,3,4-oxadiazol-2-yl}phenyl)benzamide

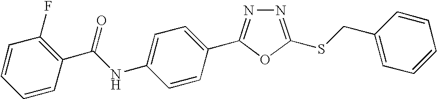

Triethylamine (2.204 mL, 15.86 mmol) was added to a suspension of 2-fluoro-N-[4-(5-thioxo-4,5-dihydro-1,3,4-oxadiazol-2-yl)phenyl]benzamide (may be prepared as described in Description 39) (5.001 g, 15.88 mmol) in ethanol (50 mL) with ice-bath cooling. After a few minutes stirring benzyl bromide (1.888 mL, 15.87 mmol) was added and then stirring continued at bath temperature for a further 5 minutes. After stirring at room temperature for 3 hours, the resulting solid was collected, washed with cold ethanol then water and finally a small volume of cold ethanol before drying under high vacuum at 40° C. The title compound was obtained as a white solid.

LC/MS (ES+ve): [M+H]⁺ at m/z 406 (C₂₂H₁₆FN₃O₂S requires [M+H]⁺ at m/z 406).

### Description 42

### 2-Fluoro-N-(3-fluoro-4-{5-[(phenylmethyl)thio]-1,3,4-oxadiazol-2-yl}phenyl)benzamide

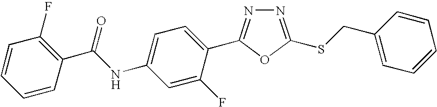

Triethylamine (1.27 mL, 9.13 mmol) was added to a suspension of 2-fluoro-N-[3-fluoro-4-(5-thioxo-4,5-dihydro-1,3,4-oxadiazol-2-yl)phenyl]benzamide (3.04 g, 9.13 mmol) in ethanol (30 mL). After stirring for 15 minutes benzyl bromide (1.09 mL, 9.13 mmol) was added and then stirred at room temperature for 2 hours. The resulting solid was filtered, washed with ethanol and ether and dried to give the title compound (3.5 g) as a white solid

LC/MS (ES+ve): [M+H]⁺ at m/z 424 (C₂₂H₁₅F₂N₃O₂S requires [M+H]⁺ at m/z 424.

The following compounds in Table 13 may be prepared in a similar manner using the appropriate thiol with variations in reactant equivalents, reaction times, work up or purifications as appropriate. In particular, sometimes the reaction is started at room temperature and other times in an ice bath. Generally 1-1.16 equivalents of benzyl bromide are used, 1-1.3 equivalents of triethylamine. The products can be purified by filtration, flash chromatography using a silica solid phase or trituration.

TABLE 13

| Structure | Name | [M + H]⁺ |
|---|---|---|
| 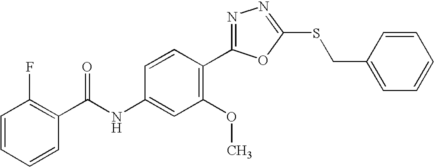 | 2-fluoro-N-(3-(methyloxy)-4-{5-[(phenylmethyl)thio]-1,3,4-oxadiazol-2-yl}phenyl)benzamide | 436 |
| 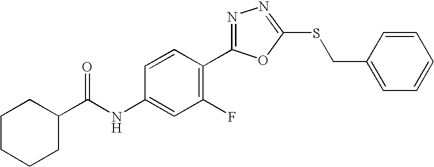 | N-(3-fluoro-4-{5-[(phenylmethyl)thio]-1,3,4-oxadiazol-2-yl}phenyl)cyclohexanecarboxamide | 412 |
| 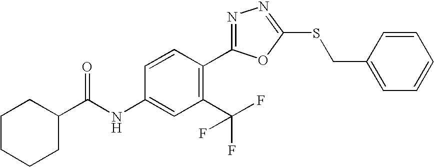 | N-[4-{5-[(phenylmethyl)thio]-1,3,4-oxadiazol-2-yl}-3-(trifluoromethyl)phenyl]cyclohexanecarboxamide | 462 |
| 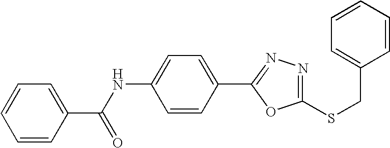 | N-(4-{5-[(phenylmethyl)thio]-1,3,4-oxadiazol-2-yl}phenyl)benzamide | 388 |
| 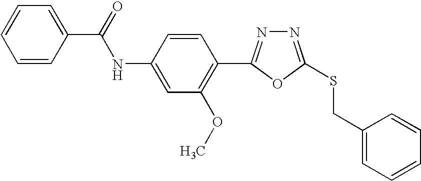 | N-(3-(methyloxy)-4-{5-[(phenylmethyl)thio]-1,3,4-oxadiazol-2-yl}phenyl)benzamide | 418 |
| 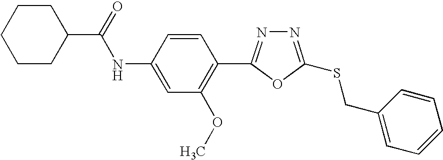 | N-(3-(methyloxy)-4-{5-[(phenylmethyl)thio]-1,3,4-oxadiazol-2-yl}phenyl)cyclohexanecarboxamide | 424 |
| 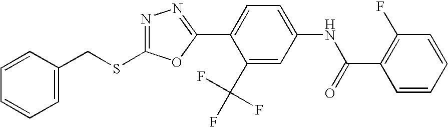 | 2-fluoro-N-[4-{5-[(phenylmethyl)thio]-1,3,4-oxadiazol-2-yl}-3-(trifluoromethyl)phenyl]benzamide | 474 |
| 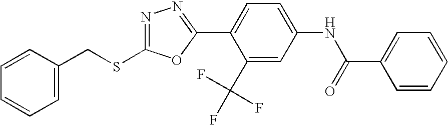 | N-[4-{5-[(phenylmethyl)thio]-1,3,4-oxadiazol-2-yl}-3-(trifluoromethyl)phenyl]benzamide | 456 |
| 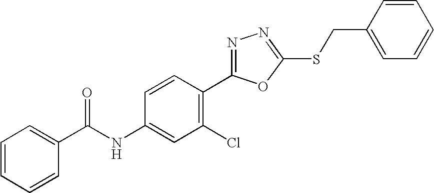 | N-(3-chloro-4-{5-[(phenylmethyl)thio]-1,3,4-oxadiazol-2-yl}phenyl)benzamide | 422, 424 |
| 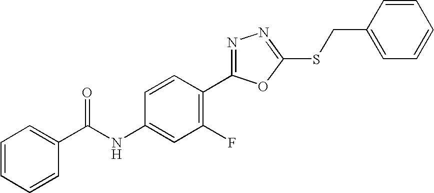 | N-(3-fluoro-4-{5-[(phenylmethyl)thio]-1,3,4-oxadiazol-2-yl}phenyl)benzamide | 406, |
| 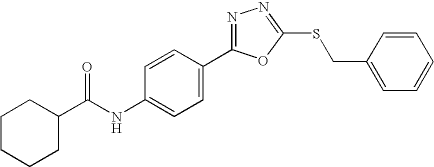 | N-(4-{5-[(phenylmethyl)thio]-1,3,4-oxadiazol-2-yl}phenyl)cyclohexanecarboxamide | 394 |
| 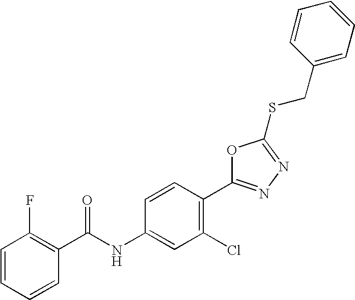 | N-(3-chloro-4-{5-[(phenylmethyl)thio]-1,3,4-oxadiazol-2-yl}phenyl)-2-fluorobenzamide | 440, 442 |
| 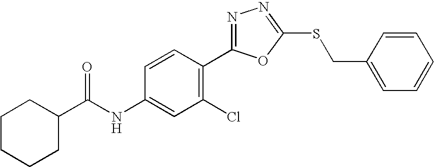 | N-(3-chloro-4-{5-[(phenylmethyl)thio]-1,3,4-oxadiazol-2-yl}phenyl)cyclohexanecarboxamide | 428, 431 |
| 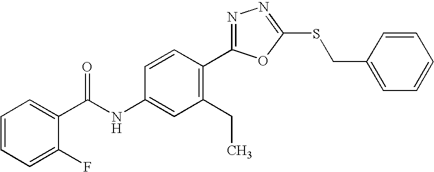 | N-(3-ethyl-4-{5-[(phenylmethyl)thio]-1,3,4-oxadiazol-2-yl}phenyl)-2-fluorobenzamide | 434 |

### Description 43

### 2-Fluoro-N-(4-{5-[(phenylmethyl)sulfonyl]-1,3,4-oxadiazol-2-yl}phenyl)benzamide

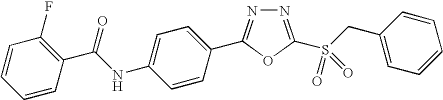

meta-Chloroperoxybenzoic acid (14.89 g, 66.44 mmol based on maximum 77% purity) was added portionwise to 2-fluoro-N-(4-{5-[(phenylmethyl)thio]-1,3,4-oxadiazol-2-yl}phenyl)benzamide (may be prepared as described in Description 41) (5.988 g, 14.77 mmol) in dichloromethane (150 mL) whilst stirring in an ice-bath. After 1 hour the bath was removed and stirring continued at room temperature for 5 days and then a further portion of meta-chloroperoxybenzoic acid (1 g) added. After stirring at room temperature overnight, the resulting white solid was collected, washed with dichloromethane (100 mL) and then further dichloromethane (60-70 mL). When analysis by LC/MS showed the mixture still contained meta-chlorobenzoic acid a portion (472 mg) of the mixture was suspended in 10% methanol in dichloromethane (10 mL) and sonicated for 1 minute. The collected solid was then free of meta-chlorobenzoic acid. The remaining crude product (5.94 g) was then treated similarly using 10% methanol/dichloromethane (125 mL), a sonication time of 2 minutes and a final wash with fresh 10% methanol/dichloromethane (10 mL) to give the title compound as a white solid.

LC/MS (ES+ve): [M+H]⁺ at m/z 438 (C₂₂H₁₆FN₃O₄S requires [M+H]⁺ at m/z 438).

### Description 44

### 2-Fluoro-N-(3-fluoro-4-{5-[(phenylmethyl)sulfonyl]-1,3,4-oxadiazol-2-yl}phenyl)benzamide

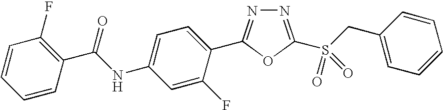

meta-Chloroperoxybenzoic acid (5.54 g, 24.81 mmol based on maximum. 77% purity) was added to a solution of 2-fluoro-N-(3-fluoro-4-{5-[(phenylmethyl)thio]-1,3,4-oxadiazol-2-yl}phenyl)benzamide (3.5 g, 8.27 mmol) in dichloromethane (35 mL) and stirred at room temperature overnight. A further portion of meta-chloroperoxybenzoic acid (1 g) added and the suspension stirred at room temperature overnight. Tetrahydrofuran (20 mL) was added to aid solubility and the suspension stirred at room temperature overnight. The precipitated solid was filtered, washed with ether and dried to give the title compound (2.0 g) as a white solid containing 15% sulphoxide, which was used without further purification.

LC/MS (ES+ve): [M+H]⁺ at m/z 456 (C₂₂H₁₅F₂N₃O₄S requires [M+H]⁺ at m/z 456).

The following compounds in Table 14 may be prepared in a similar manner using the appropriate sulfide with variations in reactant equivalents, reaction times, work up or purifications as appropriate. In particular, varying amounts of meta-chloroperoxybenzoic acid can be required and additional reagent may be required to give a good conversion, for example three followed by six equivalents. The reaction solvent may be dichloromethane or a mixture of dichloromethane and tetrahydrofuran and the reaction maybe started at room temperature or in an ice bath. Reaction times can be many days—for example five days.

TABLE 14

| Structure | Name | [M + H]⁺ |
|---|---|---|
| 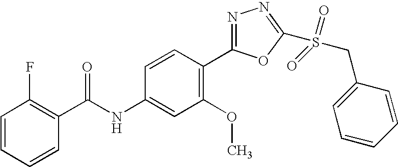 | 2-fluoro-N-(3-(methyloxy)-4-{5-[(phenylmethyl)sulfonyl]-1,3,4-oxadiazol-2-yl}phenyl)benzamide | 468 |
| 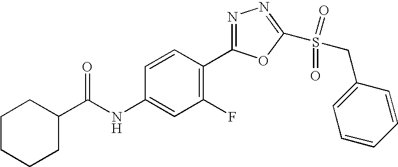 | N-(3-fluoro-4-{5-[(phenylmethyl)sulfonyl]-1,3,4-oxadiazol-2-yl}phenyl)cyclohexanecarboxamide | 444 |
| 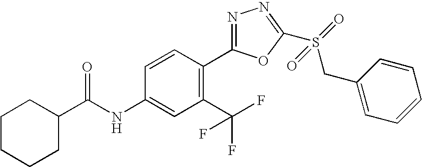 | N-[4-{5-[(phenylmethyl)sulfonyl]-1,3,4-oxadiazol-2-yl}-3-(trifluoromethyl)phenyl]cyclohexanecarboxamide | 494 |
| 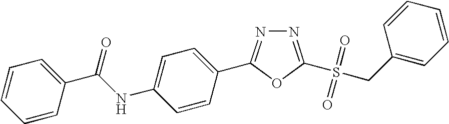 | N-(4-{5-[(phenylmethyl)sulfonyl]-1,3,4-oxadiazol-2-yl}phenyl)benzamide | 420 |
| 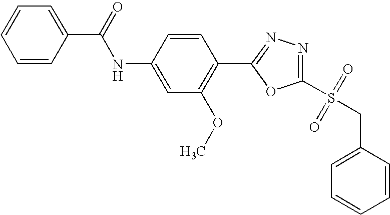 | N-(3-(methyloxy)-4-{5-[(phenylmethyl)sulfonyl]-1,3,4-oxadiazol-2-yl}phenyl)benzamide | 450 |
| 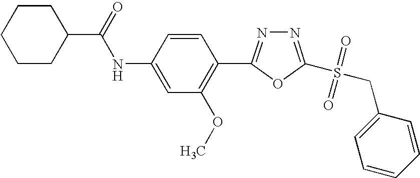 | N-(3-(methyloxy)-4-{5-[(phenylmethyl)sulfonyl]-1,3,4-oxadiazol-2-yl}phenyl)cyclohexanecarboxamide | 456 |
| 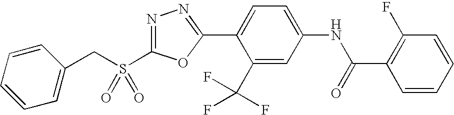 | N-(3-(methyloxy)-4-{5-[(phenylmethyl)sulfonyl]-1,3,4-oxadiazol-2-yl}phenyl)cyclohexanecarboxamide | 506 |
| 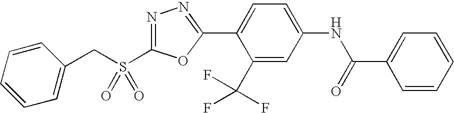 | N-[4-{5-[(phenylmethyl)sulfonyl]-1,3,4-oxadiazol-2-yl}-3-(trifluoromethyl)phenyl]benzamide | 488 |
| 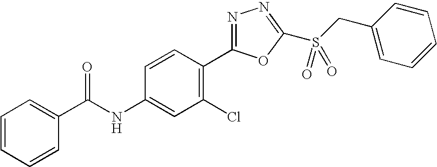 | N-(3-chloro-4-{5-[(phenylmethyl)sulfonyl]-1,3,4-oxadiazol-2-yl}phenyl)benzamide | 454, 456 |
| 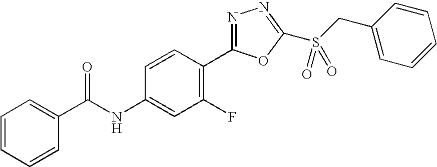 | N-(3-fluoro-4-{5-[(phenylmethyl)sulfonyl]-1,3,4-oxadiazol-2-yl}phenyl)benzamide | 438 |
| 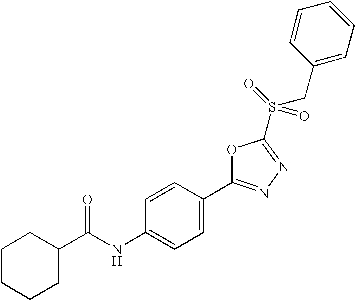 | N-(4-{5-[(phenylmethyl)sulfonyl]-1,3,4-oxadiazol-2-yl}phenyl)cyclohexanecarboxamide | 426 |
| 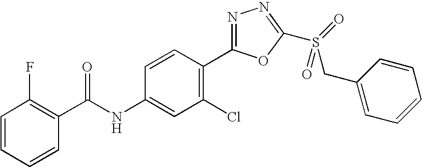 | N-(3-chloro-4-{5-[(phenylmethyl)sulfonyl]-1,3,4-oxadiazol-2-yl}phenyl)-2-fluorobenzamide | 472; 474 |
| 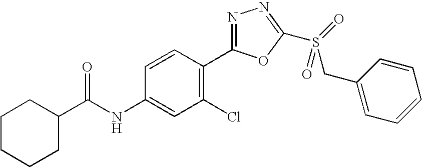 | N-(3-chloro-4-{5-[(phenylmethyl)sulfonyl]-1,3,4-oxadiazol-2-yl}phenyl)cyclohexanecarboxamide | 460 |
| 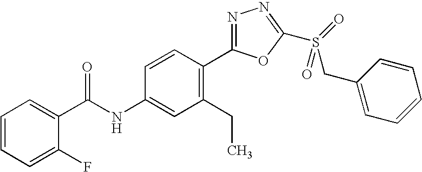 | N-(3-ethyl-4-{5-[(phenylmethyl)sulfonyl]-1,3,4-oxadiazol-2-yl}phenyl)-2-fluorobenzamide | 466 |

### Description 45

### 2-[(4-Nitrophenyl)carbonyl]-N-[3-(1-piperidinyl)propyl]hydrazinecarbothioamide

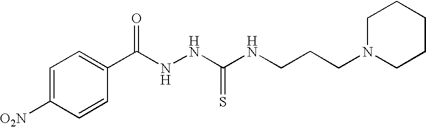

A solution of 1-(3-isothiocyanatopropyl)piperidine (may be prepared as described in Description 7, 7.63 g, 41.40 mmol) in N,N-dimethylformamide (56 mL) was added to 4-nitrobenzhydrazide (5.00 g, 27.60 mmol) in N,N-dimethylformamide (56 mL) with stirring at room temperature under an atmosphere of argon. After stirring overnight the reaction mixture was concentrated under reduced pressure and triturated using dichloromethane (100 mL). The solid was collected by filtration and dried under high vacuum to give the title compound as a dark orange/brown solid.

LC/MS (ES+ve): [M+H]⁺ at m/z 366 (C₁₆H₂₃N₅O₃S requires [M+H]⁺ at m/z 366).

### Description 46

### 2-{[2-(Ethyloxy)-4-nitrophenyl]carbonyl}-N-[3-(1-piperidinyl)propyl]hydrazinecarbothioamide

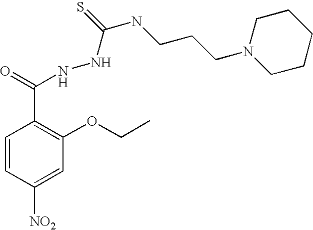

2-(Ethyloxy)-4-nitrobenzohydrazide (1.5 g, 6.67 mmol) was dissolved in tetrahydrofuran (50 mL) and 1-(3-isothiocyanatopropyl)piperidine (1.60 g, 8.67 mmol) was added and the reaction stirred at room temperature under an argon atmosphere overnight. The solvent was removed in vacuo and the product triturated with diethyl ether to give the title product (2.46 g). LCMS (ES+ve): [M+H]⁺ at m/z 410 (C₁₈H₂₇N₅O₄S requires [M+H]⁺ at m/z 410).

### Description 47

### 5-(4-Nitrophenyl)-N-[3-(1-piperidinyl)propyl]-1,3,4-oxadiazol-2-amine

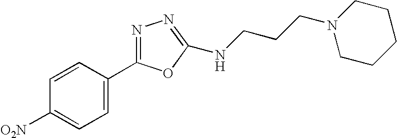

A solution of 2-[(4-nitrophenyl)carbonyl]-N-[3-(1-piperidinyl)propyl]hydrazinecarbothioamide (may be prepared as described in Description 45) (0.50 g, 1.37 mmol) and N-cyclohexylcarbodiimide, N′-methyl polystyrene (1.80 g, 3.42 mmol) in N,N-dimethylformamide (29 mL) was heated at 80° C. under an atmosphere of argon overnight. The reaction mixture was cooled to room temperature, filtered and applied to an SCX cartridge. The impurities were washed off using methanol and the product was eluted with 2M ammonia in methanol solution. The product containing fractions were combined and concentrated under reduced pressure to give the crude title compound as a dark brown oil. The crude product was purified by column chromatography [silica gel, 2M ammonia in methanol:dichloromethane (5%-10%)] to give the title compound.

LC/MS (ES+ve): [M+H]⁺ at m/z 332 (C₁₆H₂₁N₅O₃ requires [M+H]⁺ at m/z 332).

### Description 48

### 5-[2-(Ethyloxy)-4-nitrophenyl]-N-[3-(1-piperidinyl)propyl]-1,3,4-oxadiazol-2-amine

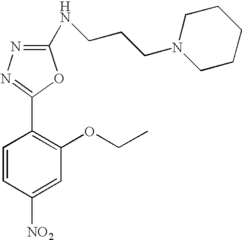

2-{[2-(Ethyloxy)-4-nitrophenyl]carbonyl}-N-[3-(1-piperidinyl)propyl]hydrazinecarbothioamide (2.46 g, 6.01 mmol) and N-[3-(dimethylamino)propyl]-N′-ethylcarbodiimide hydrochloride (1.49 g, 7.81 mmol) were stirred and heated in N,N-dimethylformamide (130 mL) at 80° C. overnight. After cooling the mixture was concentrated in vacuo and the residue partitioned between ethyl acetate (75 mL) and water (75 mL). The aqueous layer was extracted with ethyl acetate (2×30 mL) and then the combined organic phases washed with water (100 mL). The organic phase was then dried (phase separator) and concentrated in vacuo. The crude material was purified by column chromatography [silica gel, 2M ammonia in methanol:dichloromethane (5-10%)] to give the title compound (1.77 g). LCMS (ES+ve): [M+H]⁺ at m/z 376 (C₁₈H₂₅N₅O₄ requires [M+H]⁺ at m/z 376).

### Description 49

### 1,1-Dimethylethyl [5-(4-nitrophenyl)-1,3,4-oxadiazol-2-yl][3-(1-piperidinyl)propyl]carbamate

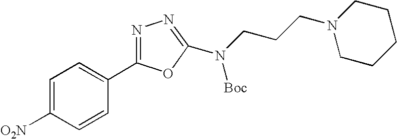

5-(4-Nitrophenyl)-N-[3-(1-piperidinyl)propyl]-1,3,4-oxadiazol-2-amine (may be prepared as described in Description 47) (2.19 g, 6.62 mmol) was dissolved in dichloromethane (11 mL), placed under an atmosphere of argon and cooled to 0° C. The reaction mixture was treated with triethylamine (0.18 mL, 1.32 mmol) and then di-tert-butyl-dicarbonate (3.61 g, 16.54 mmol). The ice-bath was later removed, and the mixture was stirred at room temperature under an atmosphere of argon for 17 hours. The reaction mixture was then concentrated under reduced pressure. The crude product was purified by column chromatography [silica gel, 2M ammonia in methanol:dichloromethane (2%-10%)] to give the title compound.

LC/MS (ES+ve): [M+H]⁺ at m/z 432 (C₂₁H₂₉N₅O₅ requires [M+H]⁺ at m/z 432).

### Description 50

### 1,1-Dimethylethyl {5-[2-(ethyloxy)-4-nitrophenyl]-1,3,4-oxadiazol-2-yl}[3-(1-piperidinyl)propyl]carbamate

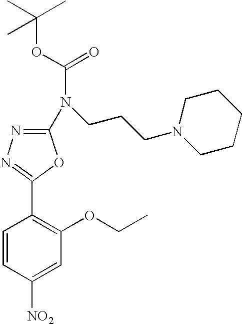

5-[2-(Ethyloxy)-4-nitrophenyl]-N-[3-(1-piperidinyl)propyl]-1,3,4-oxadiazol-2-amine (1.77 g, 4.7 mmol), di-tert-butyl-dicarbonate (2.56 g, 11.8 mmol) and triethylamine (0.65 mL, 4.7 mmol) were stirred in dichloromethane (10 mL) for about 24 h. Further triethylamine (0.65 ml, 4.7 mmol) and di-(^{t}-butyl) dicarbonate (1.02 g, 4.7 mmol) were added and the reaction stirred overnight. The solvent was removed in vacuo and the residue purified by column chromatography [silica gel, 2M ammonia in methanol:dichloromethane (5-10%)] to give the title compound (1.73 g). LCMS (ES+ve): [M+H]⁺ at m/z 476 (C₂₃H₃₃N₅O₆ requires [M+H]⁺ at m/z 476).

### Description 51

### 1,1-Dimethylethyl [5-(4-aminophenyl)-1,3,4-oxadiazol-2-yl][3-(1-piperidinyl)propyl]carbamate

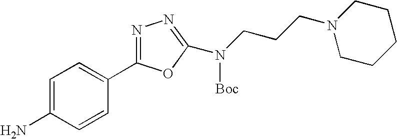

A solution of 1,1-dimethylethyl [5-(4-nitrophenyl)-1,3,4-oxadiazol-2-yl][3-(1-piperidinyl)propyl]carbamate (may be prepared as described in Description 49) (0.12 g, 0.27 mmol) and 10% Pd/C (paste) (0.11 g) in ethanol (9 mL) was hydrogenated at atmospheric pressure at room temperature over the weekend. The reaction mixture was filtered through celite and then concentrated under reduced pressure to give the title compound as a clear oil.

LC/MS (ES+ve): [M+H]⁺ at m/z 402 (C₂₁H₃₁N₅O₃ requires [M+H]⁺ at m/z 402).

### Description 52

### 1,1-Dimethylethyl {5-[4-amino-2-(ethyloxy)phenyl]-1,3,4-oxadiazol-2-yl}[3-(1-piperidinyl)propyl]carbamate

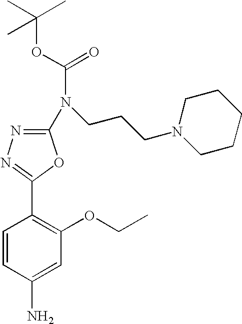

1,1-Dimethylethyl {5-[2-(ethyloxy)-4-nitrophenyl]-1,3,4-oxadiazol-2-yl}[3-(1-piperidinyl)propyl]carbamate (1.56 g, 3.29 mmol) and zinc powder (2.15 g, 32.9 mmol) were stirred in acetic acid (50 mL) overnight. The solid was removed by filtration and the solvent removed in vacuo. The residue was partitioned between dichloromethane (80 mL) and sodium bicarbonate (80 mL) with addition of aqueous sodium hydroxide (2M, 40 mL), the layers separated and the aqueous extracted with further dichloromethane. The combined organic phase was dried (phase separator) and concentrated in vacuo. The crude material was purified by column chromatography [silica gel, 2M ammonia in methanol:dichloromethane (2-6%)] to give the title compound (683 mg). LCMS (ES+ve): [M+H]⁺ at m/z 446 (C₂₃H₃₅N₅O₄) requires [M+H]⁺ at m/z 446).

### Description 53

### 1,1-Dimethylethyl (5-{4-[(phenylcarbonyl)amino]phenyl}-1,3,4-oxadiazol-2-yl)[3-(1-piperidinyl)propyl]carbamate

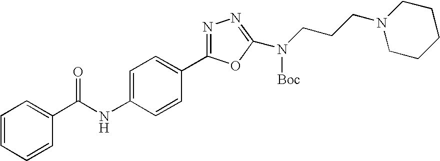

A solution of 1,1-dimethylethyl [5-(4-aminophenyl)-1,3,4-oxadiazol-2-yl][3-(1-piperidinyl)propyl]carbamate (may be prepared as described in Description 51) (0.05 g, 0.12 mmol) in dichloromethane (5 mL) was stirred at 0° C. under an atmosphere of argon. Triethylamine (0.081 mL, 0.13 mmol) and benzoyl chloride (0.015 mL, 0.13 mmol) were added and the reaction mixture was stirred at 0° C. for 45 minutes. After this time, the reaction mixture was allowed to warm to room temperature and then stirred for 45 minutes. LC/MS showed 7% starting material remaining, and so the reaction mixture was left stirring overnight. The reaction mixture was applied directly to an SCX cartridge. The impurities were washed off using methanol and the product was eluted with 2M ammonia in methanol solution. The product containing fractions were combined and concentrated to give a clear oil. The crude product was purified by column chromatography [silica gel, 2M ammonia in methanol:dichloromethane (0%-10%)] to give the title compound as a clear oil.

LC/MS (ES+ve): [M+H]⁺ at m/z 506 (C₂₈H₃₅N₅O₄ requires [M+H]⁺ at m/z 506).

The following compounds in Table 15 may be prepared in a similar manner with variations to reaction times. In some preparations the initial cooling step was not used. To prepare N-[4-(5-{[3-(1-piperidinyl)propyl]amino}-1,3,4-oxadiazol-2-yl)phenyl]-3-pyridinecarboxamide, the acid chloride hydrochloride was used, no initial cooling and an extra 0.5 equivalents of the triethylamine and the acid chloride hydrochloride were added after 22 hours and the reaction mixture was stirred for a further 11.25 hours.

TABLE 15

| Structure | Name | MH+ |
|---|---|---|
| 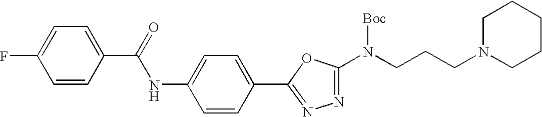 | 1,1-dimethylethyl [5-(4-{[(4-fluorophenyl)carbonyl]amino}phenyl)-1,3,4-oxadiazol-2-yl][3-(1-piperidinyl)propyl]carbamate | 524 |
| 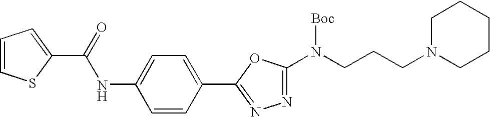 | 1,1-dimethylethyl [3-(1-piperidinyl)propyl](5-{4-[(2-thienylcarbonyl)amino]phenyl}-1,3,4-oxadiazol-2-yl)carbamate | 512 |
| 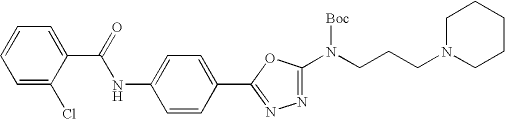 | 1,1-dimethylethyl [5-(4-{[(2-chlorophenyl)carbonyl]amino}phenyl)-1,3,4-oxadiazol-2-yl][3-(1-piperidinyl)propyl]carbamate | 540, 542 |
| 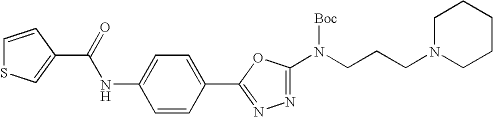 | 1,1-dimethylethyl [3-(1-piperidinyl)propyl](5-{4-[(3-thienylcarbonyl)amino]phenyl}-1,3,4-oxadiazol-2-yl)carbamate | 512 |
| 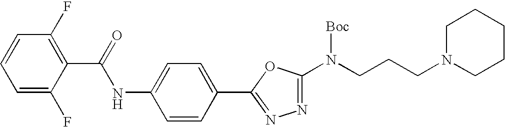 | 1,1-dimethylethyl [5-(4-{[(2,6-difluorophenyl)carbonyl]amino}phenyl)-1,3,4-oxadiazol-2-yl][3-(1-piperidinyl)propyl]carbamate | 542 |
| 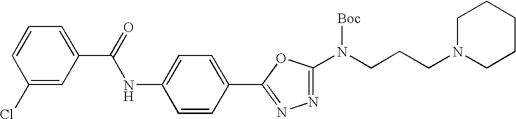 | 1,1-dimethylethyl [5-(4-{[(3-chlorophenyl)carbonyl]amino}phenyl)-1,3,4-oxadiazol-2-yl][3-(1-piperidinyl)propyl]carbamate | 540, 542 |
| 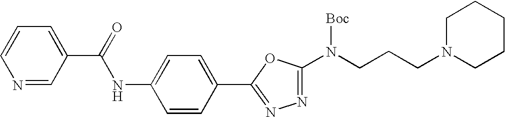 | 1,1-dimethylethyl [3-(1-piperidinyl)propyl](5-{4-[(3-pyridinylcarbonyl)amino]phenyl}-1,3,4-oxadiazol-2-yl)carbamate | 507 |
| 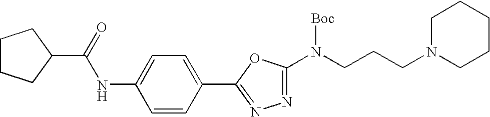 | 1,1-dimethylethyl (5-{4-[(cyclopentylcarbonyl)amino]phenyl}-1,3,4-oxadiazol-2-yl)[3-(1-piperidinyl)propyl]carbamate | 498 |
| 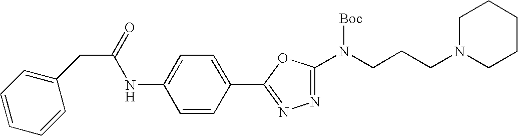 | 1,1-dimethylethyl (5-{4-[(phenylacetyl)amino]phenyl}-1,3,4-oxadiazol-2-yl)[3-(1-piperidinyl)propyl]carbamate | 520 |
| 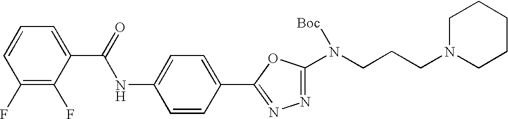 | 1,1-dimethylethyl [5-(4-{[(2,3-difluorophenyl)carbonyl]amino}phenyl)-1,3,4-oxadiazol-2-yl][3-(1-piperidinyl)propyl]carbamate | 542 |
| 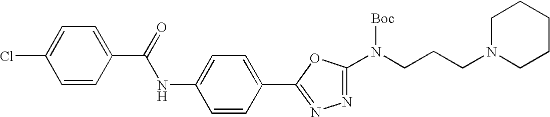 | 1,1-dimethylethyl [5-(4-{[(4-chlorophenyl)carbonyl]amino}phenyl)-1,3,4-oxadiazol-2-yl][3-(1-piperidinyl)propyl]carbamate | 540, 542 |
| 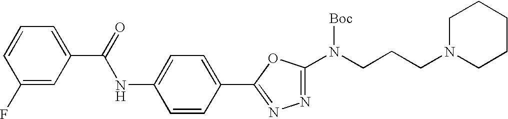 | 1,1-dimethylethyl [5-(4-{[(3-fluorophenyl)carbonyl]amino}phenyl)-1,3,4-oxadiazol-2-yl][3-(1-piperidinyl)propyl]carbamate | 524 |
| 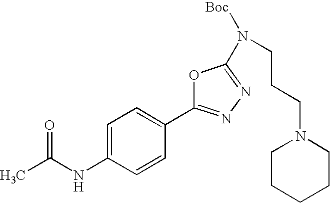 | 1,1-dimethylethyl {5-[4-(acetylamino)phenyl]-1,3,4-oxadiazol-2-yl}[3-(1-piperidinyl)propyl]carbamate | 444 |

### Description 54

### 1,1-Dimethylethyl (5-{4-[(3-furanylcarbonyl)amino]phenyl}-1,3,4-oxadiazol-2-yl)[3-(1-piperidinyl)propyl]carbamate

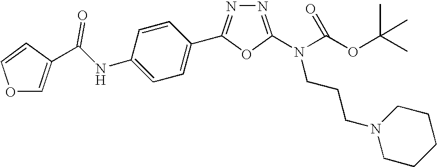

1,1-Dimethylethyl [5-(4-aminophenyl)-1,3,4-oxadiazol-2-yl][3-(1-piperidinyl)propyl]carbamate (100 mg, 0.25 mmol) was dissolved in dichloromethane (5 mL) at 0° C. and triethylamine (51 mg, 0.5 mmol) was added followed by 3-furancarbonyl chloride (65 mg, 0.50 mmol). The reaction was stirred at room temperature overnight. Further 3-furancarbonyl chloride (82 mg, 0.63 mmol) and triethylamine (57 mg, 0.56 mmol) were added and the reaction stirred for a further 24 h. The solvent was removed in vacuo to give the crude title compound. LC/MS (ES+ve): [M+H]⁺ at m/z 496 (C₂₆H₃₃N₅O₅ requires [M+H]⁺ at m/z 496.

The following compounds in Table 16 may be prepared in a similar manner using the appropriate aniline and acid chloride with variations in reactant equivalents, reaction times, work up or purifications as appropriate. It may be necessary to add additional equivalents of the acid chloride in order to improve the yield of the reaction. The products can either be used crude or purified, for example by flash chromatography with a silica support, or by using an SCX cartridge.

TABLE 16

| Structure | Name | [M + H]⁺ |
|---|---|---|
| 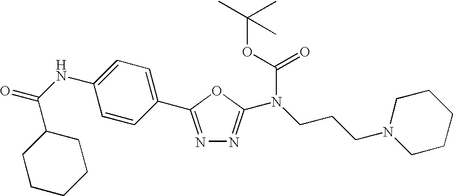 | 1,1-Dimethylethyl (5-{4-[(cyclohexylcarbonyl)amino]phenyl}-1,3,4-oxadiazol-2-yl)[3-(1-piperidinyl)propyl]carbamate | Notmeasured |
| 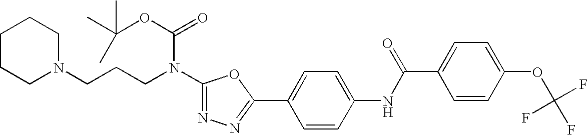 | 1,1-Dimethylethyl [3-(1-piperidinyl)propyl](5-{4-[({4-[(trifluoromethyl)oxy]phenyl}carbonyl)amino]phenyl}-1,3,4-oxadiazol-2-yl)carbamate | Notmeasured |
| 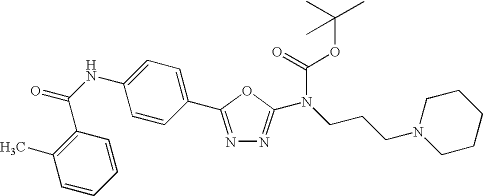 | 1,1-Dimethylethyl [5-(4-{[(2-methylphenyl)carbonyl]amino}phenyl)-1,3,4-oxadiazol-2-yl][3-(1-piperidinyl)propyl]carbamate | Notmeasured |
| 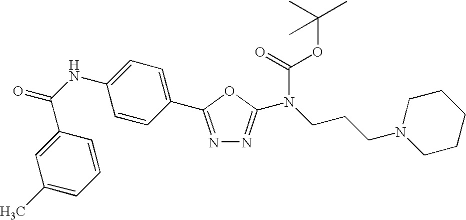 | 1,1-Dimethylethyl [5-(4-{[(3-methylphenyl)carbonyl]amino}phenyl)-1,3,4-oxadiazol-2-yl][3-(1-piperidinyl)propyl]carbamate | Notmeasured |
| 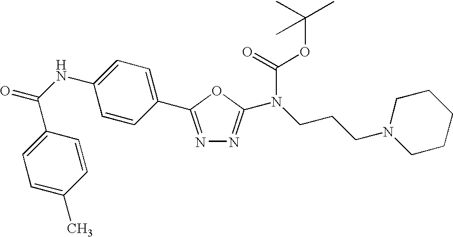 | 1,1-Dimethylethyl[5-(4-{[(4-methylphenyl)carbonyl]amino}phenyl)-1,3,4-oxadiazol-2-yl][3-(1-piperidinyl)propyl]carbamate | Notmeasured |
| 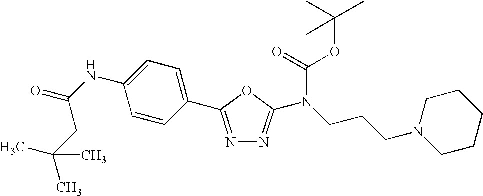 | 1,1-Dimethylethyl (5-{4-[(3,3-dimethylbutanoyl)amino]phenyl}-1,3,4-oxadiazol-2-yl)[3-(1-piperidinyl)propyl]carbamate | Notmeasured |
| 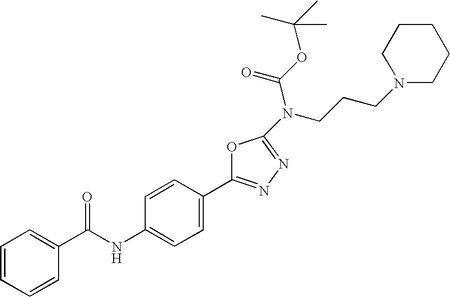 | 1,1-Dimethylethyl (5-{4-[(phenylcarbonyl)amino]phenyl}-1,3,4-oxadiazol-2-yl)[3-(1-piperidinyl)propyl]carbamate | 506 |
| 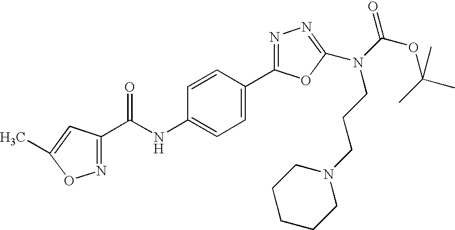 | 1,1-Dimethylethyl [5-(4-{[(5-methyl-3-isoxazolyl)carbonyl]amino}phenyl)-1,3,4-oxadiazol-2-yl][3-(1-piperidinyl)propyl]carbamate | 511 |
| 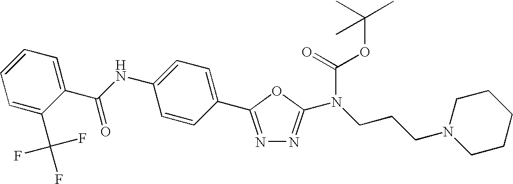 | 1,1-Dimethylethyl [3-(1-piperidinyl)propyl]{5-[4-({[2-(trifluoromethyl)phenyl]carbonyl}amino)phenyl]-1,3,4-oxadiazol-2-yl}carbamate | 574 |
| 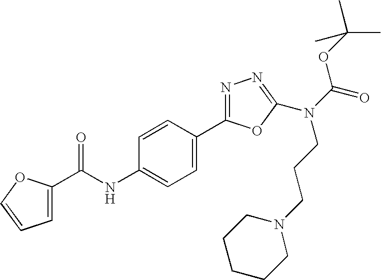 | 1,1-Dimethylethyl (5-{4-[(2-furanylcarbonyl)amino]phenyl}-1,3,4-oxadiazol-2-yl)[3-(1-piperidinyl)propyl]carbamate | 496 |
| 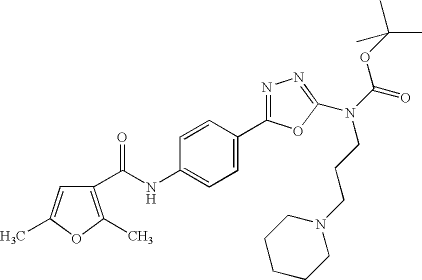 | 1,1-Dimethylethyl [5-(4-{[(2,5-dimethyl-3-furanyl)carbonyl]amino}phenyl)-1,3,4-oxadiazol-2-yl][3-(1-piperidinyl)propyl]carbamate | 524 |
| 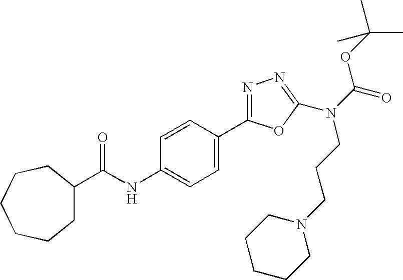 | 1,1-Dimethylethyl (5-{4-[(cycloheptylcarbonyl)amino]phenyl}-1,3,4-oxadiazol-2-yl)[3-(1-piperidinyl)propyl]carbamate | 526 |
| 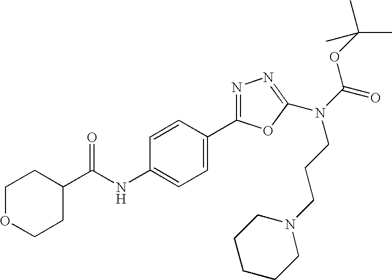 | 1,1-Dimethylethyl [3-(1-piperidinyl)propyl](5-{4-[(tetrahydro-2H-pyran-4-ylcarbonyl)amino]phenyl}-1,3,4-oxadiazol-2-yl)carbamate | 514 |
| 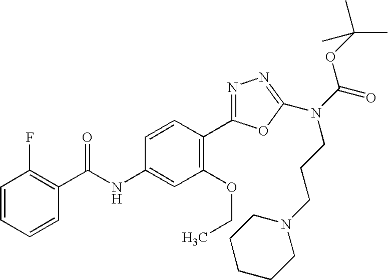 | 1,1-Dimethylethyl [5-(2-(ethyloxy)-4-{[(2-fluorophenyl)carbonyl]amino}phenyl)-1,3,4-oxadiazol-2-yl][3-(1-piperidinyl)propyl]carbamate | 568 |
| 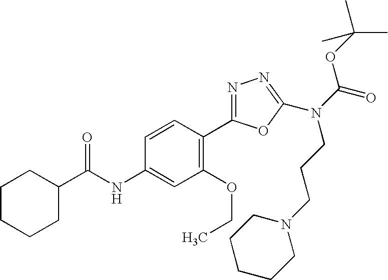 | 1,1-Dimethylethyl {5-[4-[(cyclohexylcarbonyl)amino]-2-(ethyloxy)phenyl]-1,3,4-oxadiazol-2-yl}[3-(1-piperidinyl)propyl]carbamate | 556 |
| 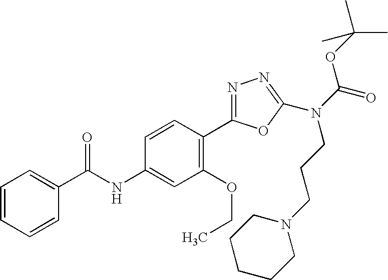 | 1,1-Dimethylethyl (5-{2-(ethyloxy)-4-[(phenylcarbonyl)amino]phenyl}-1,3,4-oxadiazol-2-yl)[3-(1-piperidinyl)propyl]carbamate | 550 |

### Description 55

### 1,1-Dimethylethyl [3-(1-piperidinyl)propyl](5-{4-[(2-pyridinylcarbonyl)amino]phenyl}-1,3,4-oxadiazol-2-yl)carbamate

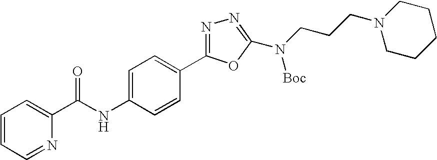

To a stirring solution of 1,1-dimethylethyl [5-(4-aminophenyl)-1,3,4-oxadiazol-2-yl][3-(1-piperidinyl)propyl]carbamate (may be prepared as described in Description 51) (75 mg, 0.187 mmol) in N,N-dimethylformamide (5 mL) was successively added N-hydroxybenzotriazole (26.5 mg, 0.20 mmol), N-cyclohexylcarbodiimide, N′-methyl polystyrene (492 mg, 1.9 mmol/g, 0.935 mmol) and 2-pyridinecarboxylic acid (24.6 mg, 0.20 mmol). The mixture stirred at room temperature under an argon atmosphere for 64.5 hours. The mixture was then filtered and the filtrate was passed down an SCX column eluting with methanol followed by 2M ammonia in methanol. The basic fractions were combined and concentrated under reduced pressure. The product was then purified by column chromatography on silica gel eluting with a mixture of 5 to 10% of 2M ammonia in methanol, in dichloromethane (12 column volumes). The title compound was obtained as a yellow solid.

LC/MS (ES+ve): [M+H]⁺ at m/z 507 (C₂₇H₃₄N₆O₄ requires [M+H]⁺ at m/z 507).

### Description 56

### Methyl-4-aminobenzoate functionalised resin

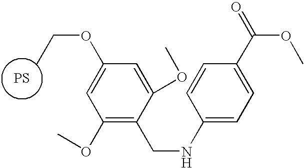

To a mixture of 4-formyl-3,5-dimethoxyphenoxy resin (Polymer Laboratories, PL-FDMP Resin, 1.82 mmol/g) and methyl-4-aminobenzoate in 10% acetic acid/N-methylpyrrolidinone was added sodium triacetoxyborohydride portionwise with mixing. The resulting mixture was shaken filtered, and washed with dimethylformamide (1×), methanol (1×), dichloromethane (1×), methanol (1×), dichloromethane (1×), and methanol (2×). The washed resin was dried in vacuo to afford the desired product.

### EXAMPLES

### Example 1

### 2-Fluoro-N-[3-(methyloxy)-4-(5-{[3-(1-piperidinyl)propyl]amino}-1,3,4-oxadiazol-2-yl)phenyl]benzamide hydrochloride

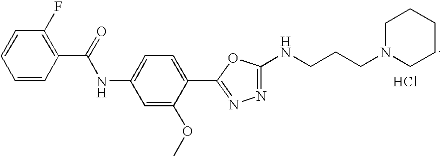

2-Fluoro-N-(3-(methyloxy)-4-{[2-({[3-(1-piperidinyl)propyl]amino}carbonothioyl)hydrazino]carbonyl}phenyl)benzamide (433 mg, 0.89 mmol) was dissolved in N,N-dimethylformamide (13 mL), treated with N-cyclohexylcarbodiimide, N′-methyl polystyrene (2.1 mmol/g, 2.12 g, 4.45 mmol) and heated at 80° C. under argon overnight. The mixture was allowed to cool to room temperature, filtered and the filtrate evaporated under reduced pressure. The product was purified on silica gel eluting with a mixture of 2M ammonia/methanol solution and dichloromethane (5:95). The resulting product was suspended in diethyl ether and the solid was collected by filtration to give the free base of the title compound. The free base 2-fluoro-N-[3-(methyloxy)-4-(5-{[3-(1-piperidinyl)propyl]amino}-1,3,4-oxadiazol-2-yl)phenyl]benzamide (230 mg, 0.51 mmol) was dissolved in methanol. A solution of hydrochloric acid in diethyl ether (1M) (0.56 mL, 0.51 mmol) was added and the mixture evaporated under reduced pressure to give the title compound as a solid.

LC/MS (ES+ve): [M+H]⁺ at m/z 454 (C₂₄H₂₈FN₅O₃ requires [M+H]⁺ at m/z 454).

TABLE 17
Examples 2-4
Examples 2-4 were prepared in an analogous manner to Example 1.

| Example | | | |
|---|---|---|---|
| Number | Structure | Name | [M + H]⁺ |
| 2 | 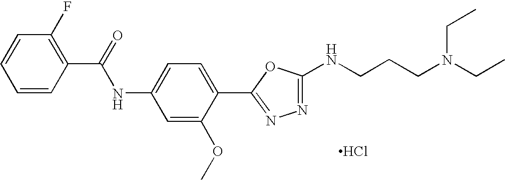 | N-[4-(5-{[3-(Diethylamino)propyl]amino}-1,3,4-oxadiazol-2-yl)-3-(methyloxy)phenyl]-2-fluorobenzamidehydrochloride | 442 |
| 3 | 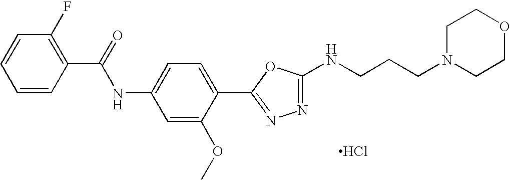 | 2-Fluoro-N-[3-(methyloxy)-4-(5-{[3-(4-morpholinyl)propyl]amino}-1,3,4-oxadiazol-2-yl)phenyl]benzamidehydrochloride | 456 |
| 4 | 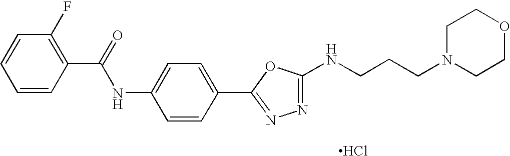 | 2-Fluoro-N-[4-(5-{[3-(4-morpholinyl)propyl]amino}-1,3,4-oxadiazol-2-yl)phenyl]benzamidehydrochloride | 426 |

### Example 5

### 2-Fluoro-N-[4-(5-{[3-(1-piperidinyl)propyl]amino}-1,3,4-oxadiazol-2-yl)phenyl]benzamide hydrochloride

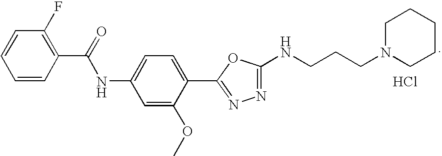

2-Fluoro-N-(4-{[2-({[3-(1-piperidinyl)propyl]amino}carbonothioyl)hydrazino]carbonyl}phenyl)benzamide (274 mg, 0.60 mmol) was dissolved in N,N-dimethylformamide (9 mL), treated with N-cyclohexylcarbodiimide, N′-methyl polystyrene (2.1 mmol/g, 1.43 g, 3.0 mmol) and heated at 80° C. under argon overnight. The mixture was allowed to cool to room temperature, filtered and the filtrate evaporated under reduced pressure. The product was purified on silica gel eluting with a mixture of 2M ammonia/methanol solution and dichloromethane (5:95) to give the free base of the title compound.

The free base 2-fluoro-N-[4-(5-{[3-(1-piperidinyl)propyl]amino}-1,3,4-oxadiazol-2-yl)phenyl]benzamide (118 mg, 0.28 mmol) was dissolved in methanol. A solution of hydrochloric acid in diethyl ether (1M) (0.31 mL, 0.31 mmol) was added and the mixture evaporated under reduced pressure to give the title compound as a solid. LC/MS (ES+ve):

### Example 6

### N-[4-(5-{[3-(1-Piperidinyl)propyl]amino}-1,3,4-oxadiazol-2-yl)phenyl]benzamide hydrochloride

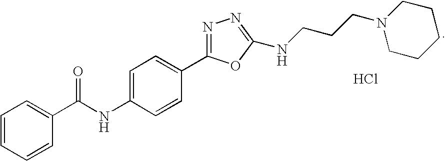

Trifluoroacetic acid (0.056 mL) was added to a stirred solution of 1,1-dimethylethyl (5-{4-[(phenylcarbonyl)amino]phenyl}-1,3,4-oxadiazol-2-yl)[3-(1-piperidinyl)propyl]carbamate (0.030 g, 0.059 mmol) in dichloromethane (1 mL) under an atmosphere of argon at room temperature. The mixture was stirred for 2 hours, and then left without stirring overnight before applying directly to an SCX cartridge. The impurities were washed off using methanol and the product was eluted with 2M ammonia in methanol. The product containing fractions were combined and concentrated under reduced pressure to give the free base of the title compound as a white solid.

The free base was treated with hydrochloric acid in diethyl ether (1M) (0.054 mL) to give the title compound as an off-white solid.

LC/MS (ES+ve): [M+H]⁺ at m/z 406 (C₂₃H₂₇N₅O₂ requires [M+H]⁺ at m/z 406).

Similarly were prepared Examples 7-20

TABLE 18
Examples 7-20

| Example | | | |
|---|---|---|---|
| Number | Structure | Name | [M + H]⁺ |
| 7 | 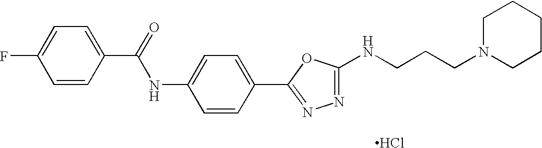 | 4-Fluoro-N-[4-(5-{[3-(1-piperidinyl)propyl]amino}-1,3,4-oxadiazol-2-yl)phenyl]benzamidehydrochloride | 424 |
| 8 | 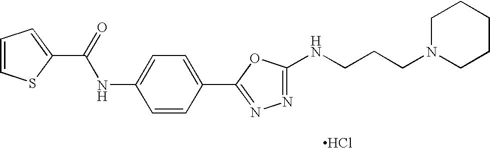 | N-[4-(5-{[3-(1-Piperidinyl)propyl]amino}-1,3,4-oxadiazol-2-yl)phenyl]-2-thiophenecarboxamidehydrochloride | 412 |
| 9 | 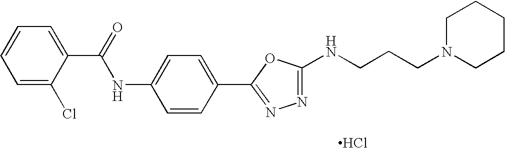 | 2-Chloro-N-[4-(5-{[3-(1-piperidinyl)propyl]amino}-1,3,4-oxadiazol-2-yl)phenyl]benzamidehydrochloride | 440, 442 |
| 10 | 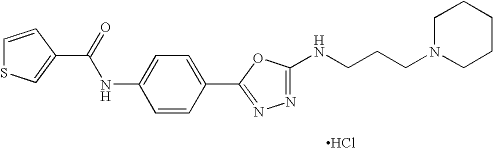 | N-[4-(5-{[3-(1-Piperidinyl)propyl]amino}-1,3,4-oxadiazol-2-yl)phenyl]-3-thiophenecarboxamidehydrochloride | 412 |
| 11 | 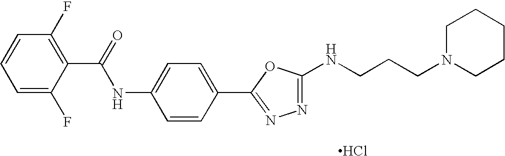 | 2,6-Difluoro-N-[4-(5-{[3-(1-piperidinyl)propyl]amino}-1,3,4-oxadiazol-2-yl)phenyl]benzamidehydrochloride | 442 |
| 12 | 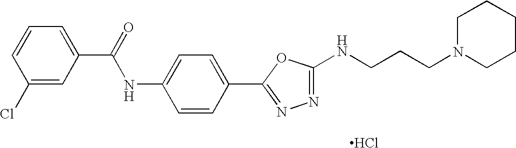 | 3-Chloro-N-[4-(5-{[3-(1-piperidinyl)propyl]amino}-1,3,4-oxadiazol-2-yl)phenyl]benzamidehydrochloride | 440, 442 |
| 13 | 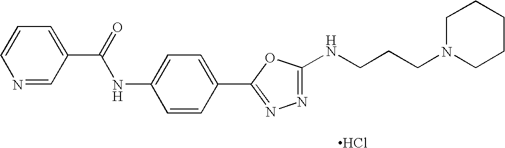 | N-[4-(5-{[3-(1-Piperidinyl)propyl]amino}-1,3,4-oxadiazol-2-yl)phenyl]-3-pyridinecarboxamidehydrochloride | 407 |
| 14 | 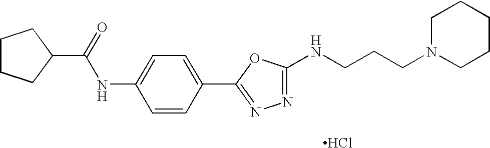 | N-[4-(5-{[3-(1-Piperidinyl)propyl]amino}-1,3,4-oxadiazol-2-yl)phenyl]cyclopentanecarboxamidehydrochloride | 398 |
| 15 | 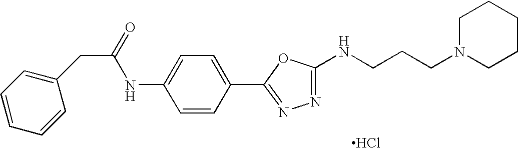 | 2-Phenyl-N-[4-(5-{[3-(1-piperidinyl)propyl]amino}-1,3,4-oxadiazol-2-yl)phenyl]acetamidehydrochloride | 420 |
| 16 | 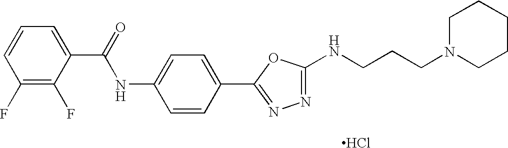 | 2,3-Difluoro-N-[4-(5-{[3-(1-piperidinyl)propyl]amino}-1,3,4-oxadiazol-2-yl)phenyl]benzamidehydrochloride | 442 |
| 17 | 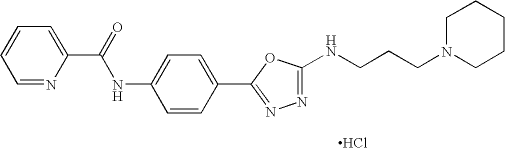 | N-[4-(5-{[3-(1-Piperidinyl)propyl]amino}-1,3,4-oxadiazol-2-yl)phenyl]-2-pyridinecarboxamidehydrochloride | 407 |
| 18 | 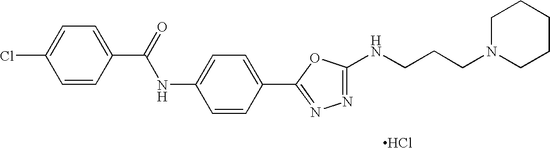 | 4-Chloro-N-[4-(5-{[3-(1-piperidinyl)propyl]amino}-1,3,4-oxadiazol-2-yl)phenyl]benzamidehydrochloride | 440, 442 |
| 19 | 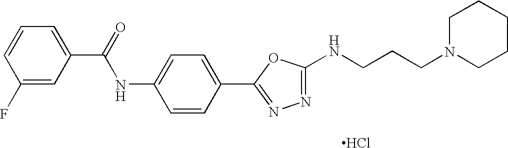 | 3-Fluoro-N-[4-(5-{[3-(1-piperidinyl)propyl]amino}-1,3,4-oxadiazol-2-yl)phenyl]benzamidehydrochloride | 424 |
| 20 | 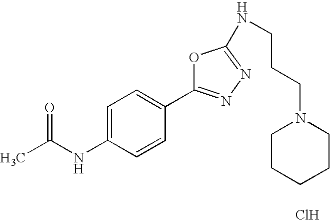 | N-[4-(5-{[3-(1-piperidinyl)propyl]amino}-1,3,4-oxadiazol-2-yl)phenyl]acetamidehydrochloride | 344 |

### Example 21

### 2-Fluoro-N-[4-(5-{[3-(1-pyrrolidinyl)propyl]amino}-1,3,4-oxadiazol-2-yl)phenyl]benzamide Hydrochloride

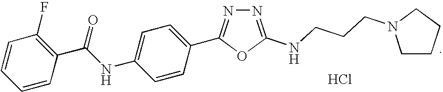

A suspension of 2-fluoro-N-(4-{5-[(phenylmethyl)sulfonyl]-1,3,4-oxadiazol-2-yl}phenyl)benzamide (0.050 g, 0.11 mmol), 1-(3-aminopropyl)pyrrolidine (0.034 mL, 0.28 mmol) in 1,4 dioxane (2 mL) was heated in the microwave at 100° C. for 15 minutes. The reaction mixture was concentrated under reduced pressure to give a clear oil. The crude product was purified by column chromatography [silica gel, 2M ammonia in methanol:dichloromethane (5%-10%)] to give the free base of the title compound as a clear oil.

The free base was treated with hydrochloric acid in diethyl ether (1M) (0.080 mL) to give the title compound as an off-white solid.

LC/MS (ES+ve): [M+H]⁺ at m/z 410 (C₂₂H₂₄FN₅O₂ requires [M+H]⁺ at m/z 410).

Similarly were prepared Examples 22-24.

TABLE 19
Examples 22-24

| Example | | | |
|---|---|---|---|
| Number | Structure | Name | [M + H]⁺ |
| 22 | 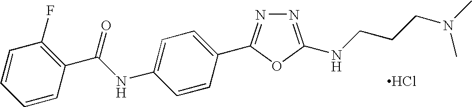 | N-[4-(5-{[3-(Dimethylamino)propyl]amino}-1,3,4-oxadiazol-2-yl)phenyl]-2-fluorobenzamidehydrochloride | 384 |
| 23 | 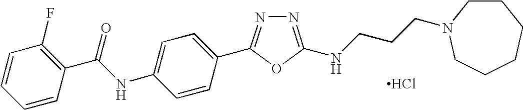 | 2-Fluoro-N-[4-(5-{[3-(hexahydro-1H-azepin-1-yl)propyl]amino}-1,3,4-oxadiazol-2-yl)phenyl]benzamidehydrochloride | 438 |
| 24 | 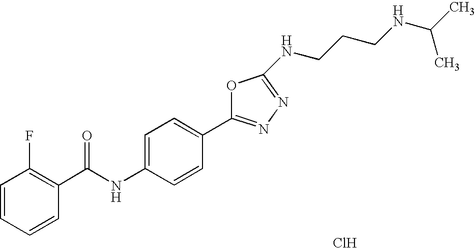 | 2-fluoro-N-{4-[5-({3-[(1-methylethyl)amino]propyl}amino)-1,3,4-oxadiazol-2-yl]phenyl}benzamidehydrochloride | 398 |

### Example 25

### 2-Fluoro-N-[3-fluoro-4-(5-{[4-(4-morpholinyl)butyl]amino}-1,3,4-oxadiazol-2-yl)phenyl]benzamide

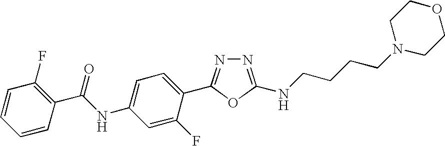

A suspension of 2-fluoro-N-(3-fluoro-4-{5-[(phenylmethyl)sulfonyl]-1,3,4-oxadiazol-2-yl}phenyl)benzamide (2×1 g, 2.2 mmol) and 1-(4-aminobutyl)morpholine (2×0.87 g, 5.5 mmol) in 1,4 dioxane (2×15 mL) was heated in the microwave at 100° C. for 15 minutes. The reaction mixtures were combined, diluted with ethyl acetate, washed with water (3×) dried (magnesium sulphate) and evaporated. The crude product was purified by column chromatography [silica gel, 2M ammonia in methanol:dichloromethane (4%)] and triturated with ether to give the title compound (1.27 g) as a white solid.

LC/MS (ES+ve): [M+H]⁺ at m/z 458 (C₂₃H₂₅F₂N₅O₃ requires [M+H]⁺ at m/z 458).

### Example 26

### 2-Fluoro-N-[3-fluoro-4-(5-{[4-(4-fluoro-1-piperidinyl)butyl]amino}-1,3,4-oxadiazol-2-yl)phenyl]benzamide

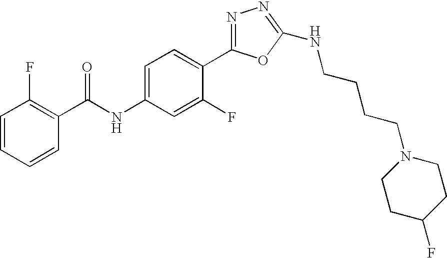

[4-(4-Fluoro-1-piperidinyl)butyl]amine (225 mg, 1.29 mmol) and 1,1-thiocarbonyldiimidazole (230 mg, 1.29 mmol) were dissolved in N,N-dimethylformamide (15 mL) and stirred at room temperature overnight. A third of the solution (5 mL) was added to 2-fluoro-N-[3-fluoro-4-(hydrazinocarbonyl)phenyl]benzamide (125 mg, 0.43 mmol) and the reaction stirred overnight. To the reaction mixture was then added N-[3-(dimethylamino)propyl]-N′-ethylcarbodiimide hydrochloride (165 mg, 0.86 mmol) and the reaction heated at 65° C. for 21 h. The reaction mixture was partioned between aqueous sodium hydroxide (2M, 15 mL) and ethyl acetate (20 mL). The organic phase was washed with further sodium hydroxide (10 mL) and water (2×15 mL) before it was separated and concentrated in vacuo. The residue was triturated with acetone to afford the title product (69 mg) as a white solid LC/MS (ES+ve): [M+H]⁺ at m/z 474 (C₂₄H₂₆F₃N₅O₂ requires [M+H]⁺ at m/z 474.

### Example 27

### 2-Fluoro-N-[4-(5-{[3-(4-fluoro-1-piperidinyl)propyl]amino}-1,3,4-oxadiazol-2-yl)phenyl]benzamide

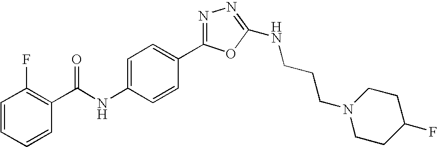

4-Fluoropiperidine hydrochloride (164 mg, 1.18 mmol) was partitioned between dichloromethane (5 mL) and aqueous sodium hydroxide (2M, 5 mL) and the aqueous phase extracted with further dichloromethane (5 mL). The organic phases were combined and isolated (phase separator). To this solution was added 3-(1,3-dioxo-1,3-dihydro-2H-isoindol-2-yl)propanal (300 mg, 1.48 mmol) and sodium triacetoxyborohydride (470 mg, 2.22 mmol) and then the reaction was stirred at room temperature overnight. Methanol was added to the reaction mixture which was applied to an SCX cartridge (10 g), washed with methanol and eluted with 2M ammonia in methanol. The solvent was removed in vacuo and the residue was taken up in a solution of methylamine in ethanol (33%, 2 mL) and the reaction stirred at room temperature overnight. Diethyl ether (7 mL) was then added and the resultant solid removed by filtration. The solvent was removed in vacuo and the crude amine, [3-(4-fluoro-1-piperidinyl)propyl]amine, then taken up in dioxane (3 mL) and 2-fluoro-N-(4-{5-[(phenylmethyl)sulfonyl]-1,3,4-oxadiazol-2-yl}phenyl)benzamide (70 mg, 0.16 mmol) was added. The reaction was heated in the microwave at 100° C. for 15 minutes. The solvent was removed in vacuo and the residue purified by mass directed auto-preparative liquid chromatography. The product was applied to an SCX cartridge, washed with methanol and eluted with 2M ammonia in methanol. The solvent was removed in vacuo to furnish the title product (45 mg) as a white solid. LCMS (ES+ve): [M+H]⁺ at m/z 442 (C₂₃H₂₅F₂N₅O₂ requires [M+H]⁺ at m/z 442.

Compounds of Examples 28 to 44 were prepared in a similar fashion to Example 1. Variations to the procedure used in Example 1 include, but are not limited to the use of EDAC.HCl rather than N-cyclohexylcarbodiimide, N′-methylpolystyrene, the molar equivalents to the starting material used of EDAC.HCl or N-cyclohexylcarbodiimide, N′-methylpolystyrene, the reaction time used, the temperature used. The purification methods used were selected from: chromatography using EtOAc/hexane, NH₃/MeOH/DCM, MeOH/DCM, or recrystallisation from ethanol. In certain Examples, chromatography was followed by trituration with ether. Example 38 was purified using MDAP then freeze dried. Examples 39 and 40 were triturated without chromatography.

TABLE 20
Examples 28 to 44

| Example | | | |
|---|---|---|---|
| Number | Structure | Name | [M + H]+ |
| 28 | 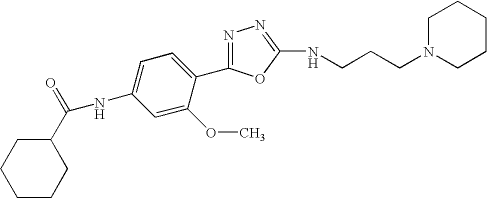 | N-[3-(methyloxy)-4-(5-{[3-(1-piperidinyl)propyl]amino}-1,3,4-oxadiazol-2-yl)phenyl]cyclohexanecarboxamide | 442 |
| 29 | 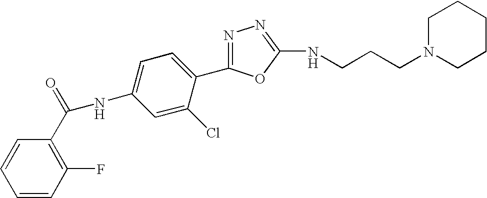 | N-[3-chloro-4-(5-{[3-(1-piperidinyl)propyl]amino}-1,3,4-oxadiazol-2-yl)phenyl]-2-fluorobenzamide | 458 |
| 30 | 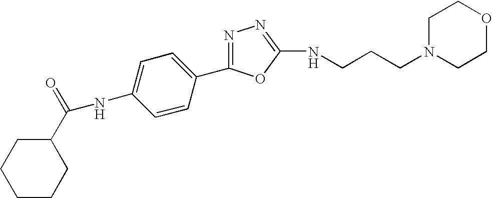 | N-[4-(5-{[3-(4-morpholinyl)propyl]amino}-1,3,4-oxadiazol-2-yl)phenyl]cyclohexanecarboxamide | 414 |
| 31 | 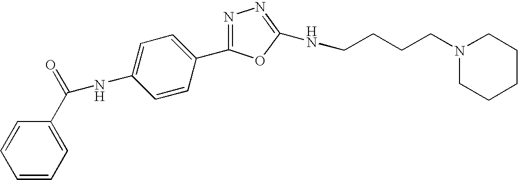 | N-[4-(5-{[4-(1-piperidinyl)butyl]amino}-1,3,4-oxadiazol-2-yl)phenyl]benzamide | 420 |
| 32 | 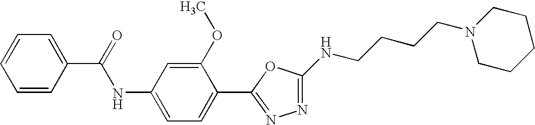 | N-[3-(methyloxy)-4-(5-{[4-(1-piperidinyl)butyl]amino}-1,3,4-oxadiazol-2-yl)phenyl]benzamide | 450 |
| 33 | 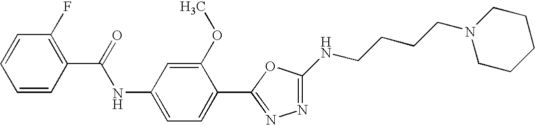 | 2-fluoro-N-[3-(methyloxy)-4-(5-{[4-(1-piperidinyl)butyl]amino}-1,3,4-oxadiazol-2-yl)phenyl]benzamide | 468 |
| 34 | 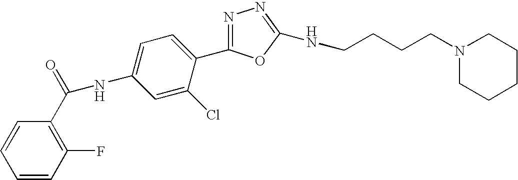 | N-[3-chloro-4-(5-{[4-(1-piperidinyl)butyl]amino}-1,3,4-oxadiazol-2-yl)phenyl]-2-fluorobenzamide | 472 |
| 35 | 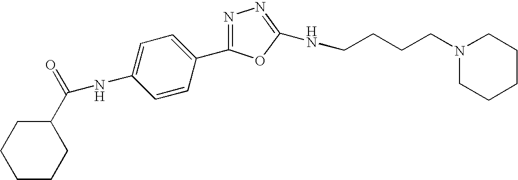 | N-[4-(5-{[4-(1-piperidinyl)butyl]amino}-1,3,4-oxadiazol-2-yl)phenyl]cyclohexanecarboxamide | 426 |
| 36 | 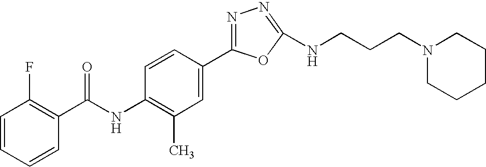 | 2-fluoro-N-[2-methyl-4-(5-{[3-(1-piperidinyl)propyl]amino}-1,3,4-oxadiazol-2-yl)phenyl]benzamide | 438 |
| 37 | 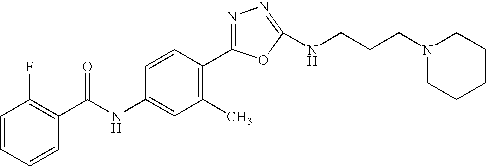 | 2-fluoro-N-[3-methyl-4-(5-{[3-(1-piperidinyl)propyl]amino}-1,3,4-oxadiazol-2-yl)phenyl]benzamide | 438 |
| 38 | 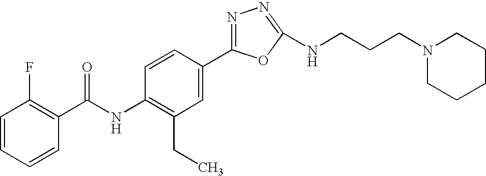 | N-[2-ethyl-4-(5-{[3-(1-piperidinyl)propyl]amino}-1,3,4-oxadiazol-2-yl)phenyl]-2-fluorobenzamide | 452 |
| 39 | 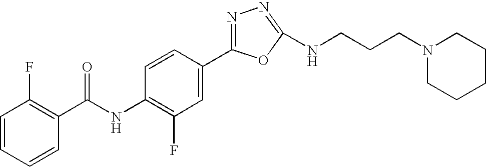 | 2-fluoro-N-[2-fluoro-4-(5-{[3-(1-piperidinyl)propyl]amino}-1,3,4-oxadiazol-2-yl)phenyl]benzamide | 442 |
| 40 | 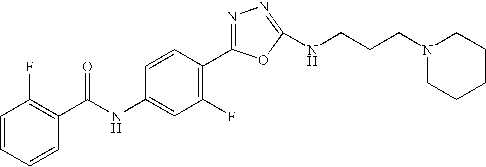 | 2-fluoro-N-[3-fluoro-4-(5-{[3-(1-piperidinyl)propyl]amino}-1,3,4-oxadiazol-2-yl)phenyl]benzamide | 442 |
| 41 | 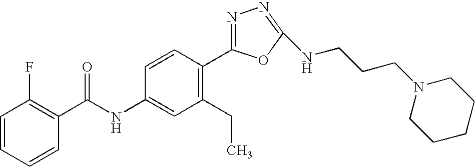 | N-[3-ethyl-4-(5-{[3-(1-piperidinyl)propyl]amino}-1,3,4-oxadiazol-2-yl)phenyl]-2-fluorobenzamide | 452 |
| 42 | 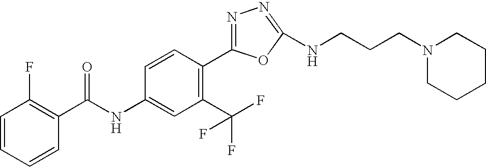 | 2-fluoro-N-[4-(5-{[3-(1-piperidinyl)propyl]amino}-1,3,4-oxadiazol-2-yl)-3-(trifluoromethyl)phenyl]benzamide | 492 |
| 43 | 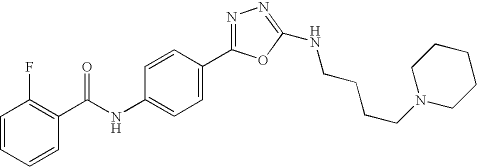 | 2-fluoro-N-[4-(5-{[4-(1-piperidinyl)butyl]amino}-1,3,4-oxadiazol-2-yl)phenyl]benzamide | 438 |
| 44 | 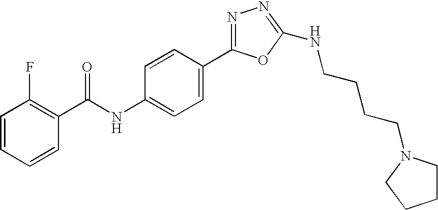 | 2-fluoro-N-[4-(5-{[4-(1-pyrrolidinyl)butyl]amino}-1,3,4-oxadiazol-2-yl)phenyl]benzamide | 424 |

Example 45 was prepared in a similar fashion to Example 26 apart from the purification which was carried out by MDAP followed by trituration with ether.

| Example | | | |
|---|---|---|---|
| Number | Structure | Name | [M + H]+ |
| 45 | 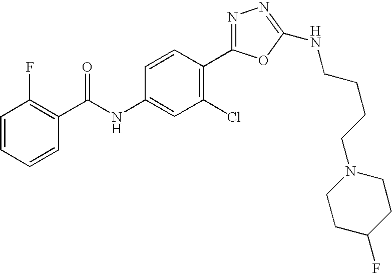 | N-[3-chloro-4-(5-{[4-(4-fluoro-1-piperidinyl)butyl]amino}-1,3,4-oxadiazol-2-yl)phenyl]-2-fluorobenzamide | 490 |

Compounds of Examples 46-134, and 145 to 176 were prepared in a similar fashion to Example 21. The compound of Example 44 was prepared in a similar manner to Example 21 in addition to being prepared in a similar manner to Example 1.

The method for Example 27 was used for Examples 135-144.

Some of the amines were available as the free base and in these case the separation between DCM and NaOH was not required.

Variations to the procedure used in Example 21 include but are not limited to the molar equivalents, the reaction time used and the purification method. In some cases Examples were heated thermally rather than in the microwave.

TABLE 21
Examples 46 to 215

| Example | | | |
|---|---|---|---|
| Number | Structure | Name | [M + H]+ |
| 46 | 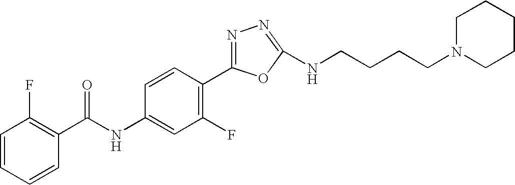 | 2-fluoro-N-[3-fluoro-4-(5-{[4-(1-piperidinyl)butyl]amino}-1,3,4-oxadiazol-2-yl)phenyl]benzamide | 456 |
| 47 | 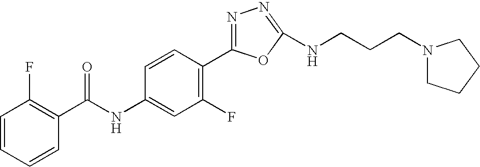 | 2-fluoro-N-[3-fluoro-4-(5-{[3-(1-pyrrolidinyl)propyl]amino}-1,3,4-oxadiazol-2-yl)phenyl]benzamide | 428 |
| 48 | 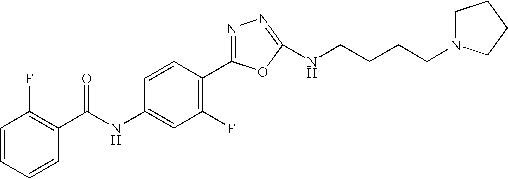 | 2-fluoro-N-[3-fluoro-4-(5-{[4-(1-pyrrolidinyl)butyl]amino}-1,3,4-oxadiazol-2-yl)phenyl]benzamide | 442 |
| 49 | 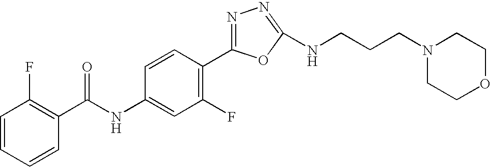 | 2-fluoro-N-[3-fluoro-4-(5-{[3-(4-morpholinyl)propyl]amino}-1,3,4-oxadiazol-2-yl)phenyl]benzamide | 444 |
| 50 | 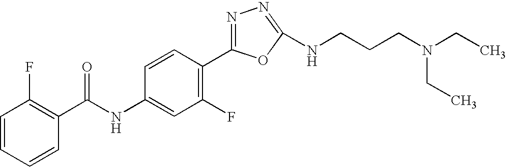 | N-[4-(5-{[3-(diethylamino)propyl]amino}-1,3,4-oxadiazol-2-yl)-3-fluorophenyl]-2-fluorobenzamide | 430 |
| 51 | 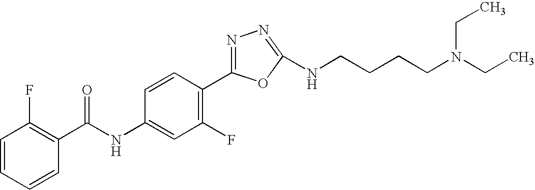 | N-[4-(5-{[4-(diethylamino)butyl]amino}-1,3,4-oxadiazol-2-yl)-3-fluorophenyl]-2-fluorobenzamide | 444 |
| 52 | 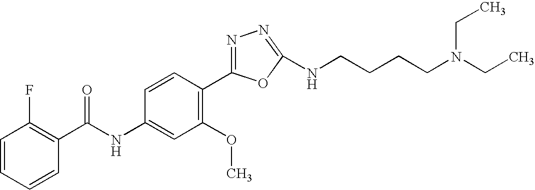 | N-[4-(5-{[4-(diethylamino)butyl]amino}-1,3,4-oxadiazol-2-yl)-3-(methyloxy)phenyl]-2-fluorobenzamide | 456 |
| 53 | 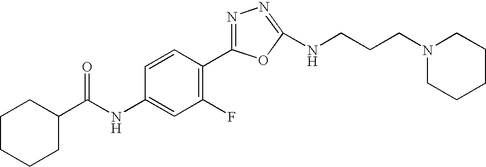 | N-[3-fluoro-4-(5-{[3-(1-piperidinyl)propyl]amino}-1,3,4-oxadiazol-2-yl)phenyl]cyclohexanecarboxamide | 430 |
| 54 | 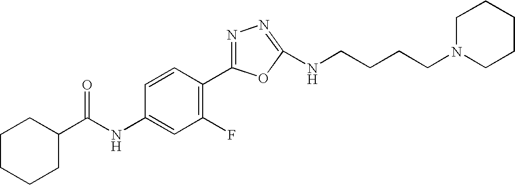 | N-[3-fluoro-4-(5-{[4-(1-piperidinyl)butyl]amino}-1,3,4-oxadiazol-2-yl)phenyl]cyclohexanecarboxamide | 444 |
| 55 | 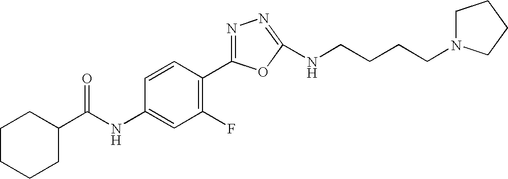 | N-[3-fluoro-4-(5-{[4-(1-pyrrolidinyl)butyl]amino}-1,3,4-oxadiazol-2-yl)phenyl]cyclohexanecarboxamide | 430 |
| 56 | 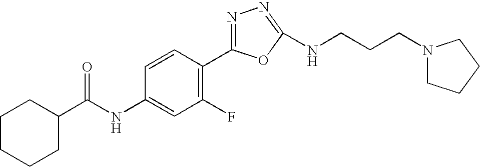 | N-[3-fluoro-4-(5-{[3-(1-pyrrolidinyl)propyl]amino}-1,3,4-oxadiazol-2-yl)phenyl]cyclohexanecarboxamide | 416 |
| 57 | 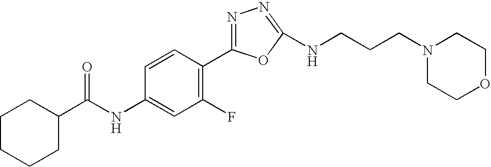 | N-[3-fluoro-4-(5-{[3-(4-morpholinyl)propyl]amino}-1,3,4-oxadiazol-2-yl)phenyl]cyclohexanecarboxamide | 432 |
| 58 | 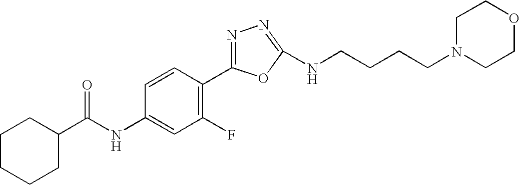 | N-[3-fluoro-4-(5-{[4-(4-morpholinyl)butyl]amino}-1,3,4-oxadiazol-2-yl)phenyl]cyclohexanecarboxamide | 446 |
| 59 | 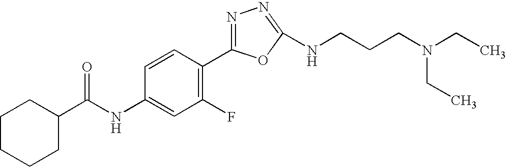 | N-[4-(5-{[3-(diethylamino)propyl]amino}-1,3,4-oxadiazol-2-yl)-3-fluorophenyl]cyclohexanecarboxamide | 418 |
| 60 | 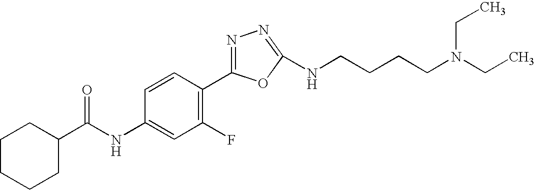 | N-[4-(5-{[4-(diethylamino)butyl]amino}-1,3,4-oxadiazol-2-yl)-3-fluorophenyl]cyclohexanecarboxamide | 432 |
| 61 | 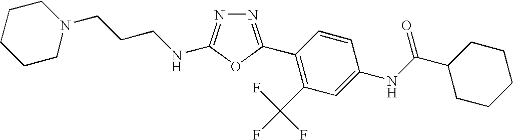 | N-[4-(5-{[3-(1-piperidinyl)propyl]amino}-1,3,4-oxadiazol-2-yl)-3-(trifluoromethyl)phenyl]cyclohexanecarboxamide | 480 |
| 62 | 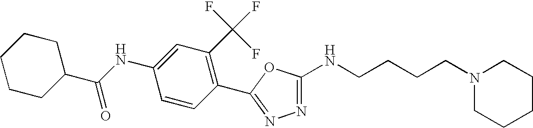 | N-[4-(5-{[4-(1-piperidinyl)butyl]amino}-1,3,4-oxadiazol-2-yl)-3-(trifluoromethyl)phenyl]cyclohexanecarboxamide | 494 |
| 63 | 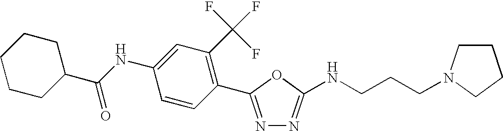 | N-[4-(5-{[3-(1-pyrrolidinyl)propyl]amino}-1,3,4-oxadiazol-2-yl)-3-(trifluoromethyl)phenyl]cyclohexanecarboxamide | 466 |
| 64 | 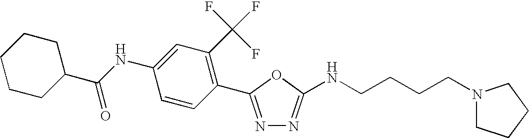 | N-[4-(5-{[4-(1-pyrrolidinyl)butyl]amino}-1,3,4-oxadiazol-2-yl)-3-(trifiuoromethyl)phenyl]cyclohexanecarboxamide | 480 |
| 65 | 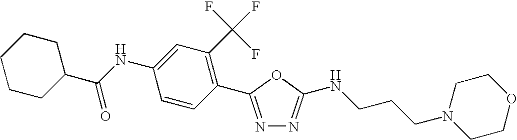 | N-[4-(5-{[3-(4-morpholinyl)propy]amino}-1,3,4-oxadiazol-2-yl)-3-(trifluoromethyl)phenyl]cyclohexanecarboxamide | 482 |
| 66 | 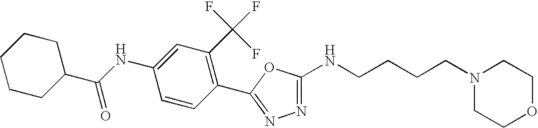 | N-[4-(5-{[4-(4-morpholinyl)butyl]amino}-1,3,4-oxadiazol-2-yl)-3-(trifluoromethyl)phenyl]cyclohexanecarboxamide | 496 |
| 67 | 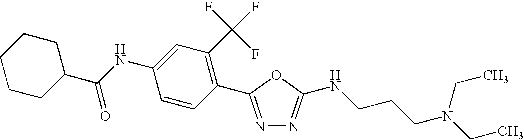 | N-[4-(5-{[3-(diethylamino)propyl]amino}-1,3,4-oxadiazol-2-yl)-3-(trifluoromethyl)phenyl]cyclohexanecarboxamide | 468 |
| 68 | 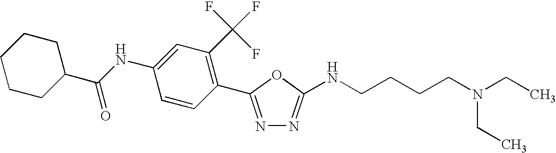 | N-[4-(5-{[4-(diethylamino)butyl]amino}-1,3,4-oxadiazol-2-yl)-3-(trifluoromethyl)phenyl]cyclohexanecarboxamide | 482 |
| 69 | 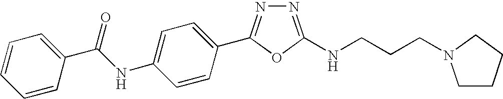 | N-[4-(5-{[3-(1-pyrrolidinyl)propyl]amino}-1,3,4-oxadiazol-2-yl)phenyl]benzamide | 392 |
| 70 | 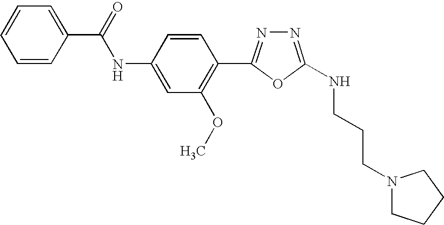 | N-[3-(methyloxy)-4-(5-{[3-(1-pyrrolidinyl)propyl]amino}-1,3,4-oxadiazol-2-yl)phenyl]benzamide | 422 |
| 71 | 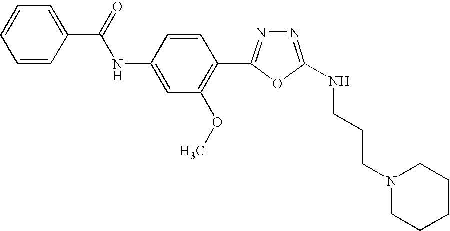 | N-[3-(methyloxy)-4-(5-{[3-(1-piperidinyl)propyl]amino}-1,3,4-oxadiazol-2-yl)phenyl]benzamide | 436 |
| 72 | 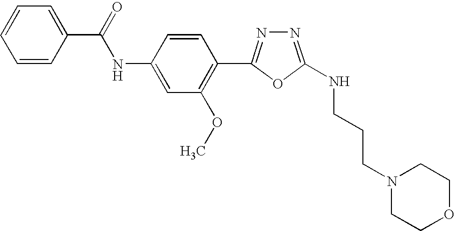 | N-[3-(methyloxy)-4-(5-{[3-(4-morpholinyl)propyl]amino}-1,3,4-oxadiazol-2-yl)phenyl]benzamide | 438 |
| 73 | 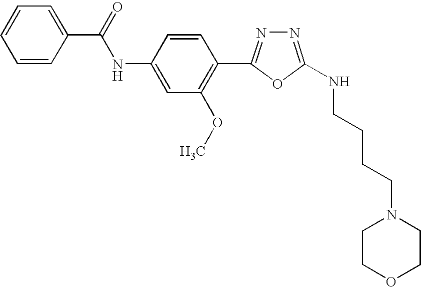 | N-[3-(methyloxy)-4-(5-{[4-(4-morpholinyl)butyl]amino}-1,3,4-oxadiazol-2-yl)phenyl]benzamide | 452 |
| 74 | 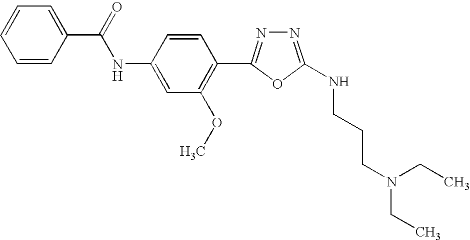 | N-[4-(5-{[3-(diethylamino)propyl]amino}-1,3,4-oxadiazol-2-yl)-3-(methyloxy)phenyl]benzamide | 424 |
| 75 | 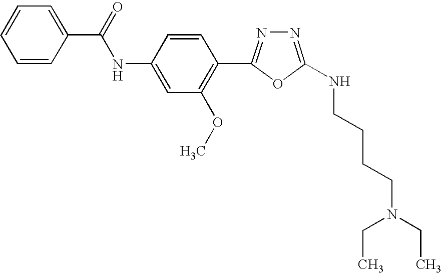 | N-[4-(5-{[4-(diethylamino)butyl]amino}-1,3,4-oxadiazol-2-yl)-3-(methyloxy)phenyl]benzamide | 438 |
| 76 | 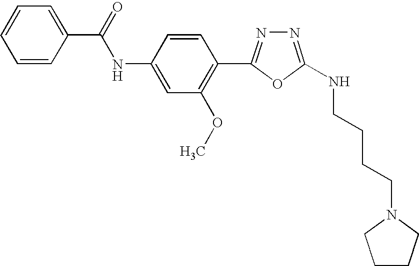 | N-[3-(methyloxy)-4-(5-{[4-(1-pyrrolidinyl)butyl]amino}-1,3,4-oxadiazol-2-yl)phenyl]benzamide | 436 |
| 77 | 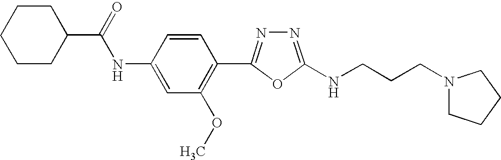 | N-[3-(methyloxy)-4-(5-{[3-(1-pyrrolidinyl)propyl]amino}-1,3,4-oxadiazol-2-yl)phenyl]cyclohexanecarboxamide | 428 |
| 78 | 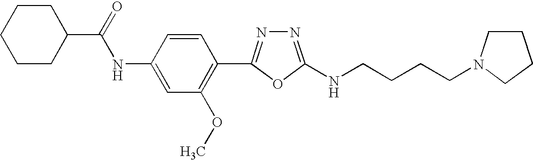 | N-[3-(methyloxy)-4-(5-{[4-(1-pyrrolidinyl)butyl]amino}-1,3,4-oxadiazol-2-yl)phenyl]cyclohexanecarboxamide | 442 |
| 79 | 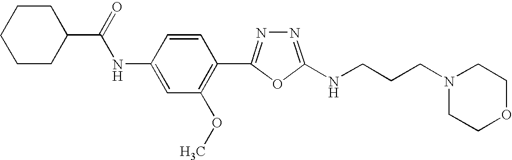 | N-[3-(methyloxy)-4-(5-{[3-(4-morpholinyl)propyl]amino}-1,3,4-oxadiazol-2-yl)phenyl]cyclohexanecarboxamide | 444 |
| 80 | 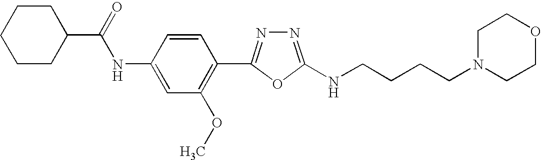 | N-[3-(methyloxy)-4-(5-{[4-(4-morpholinyl)butyl]amino}-1,3,4-oxadiazol-2-yl)phenyl]cyclohexanecarboxamide | 458 |
| 81 | 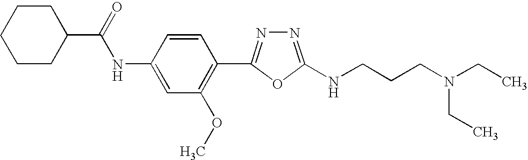 | N-[4-(5-{[3-(diethylamino)propyl]amino}-1,3,4-oxadiazol-2-yl)-3-(methyloxy)phenyl]cyclohexanecarboxamide | 430 |
| 82 | 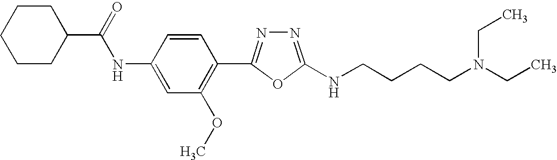 | N-[4-(5-{[4-(diethylamino)butyl]amino}-1,3,4-oxadiazol-2-yl)-3-(methyloxy)phenyl]cyclohexanecarboxamide | 444 |
| 83 | 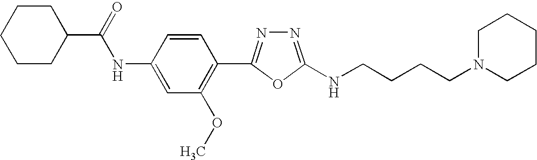 | N-[3-(methyloxy)-4-(5-{[4-(1-piperidinyl)butyl]amino}-1,3,4-oxadiazol-2-yl)phenyl]cyclohexanecarboxamide | 456 |
| 84 | 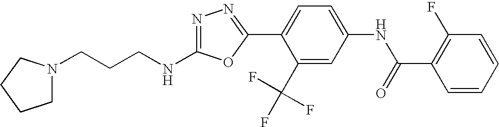 | 2-fluoro-N-[4-(5-{[3-(1-pyrrolidinyl)propyl]amino}-1,3,4-oxadiazol-2-yl)-3-(trifluoromethyl)phenyl]benzamide | 478 |
| 85 | 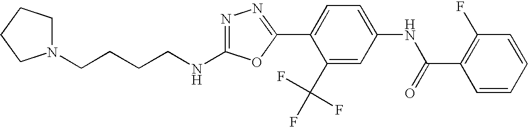 | 2-fluoro-N-[4-(5-{[4-(1-pyrrolidinyl)butyl]amino}-1,3,4-oxadiazol-2-yl)-3-(trifluoromethyl)phenyl]benzamide | 492 |
| 86 | 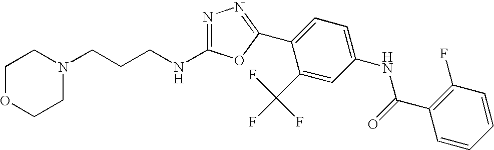 | 2-fluoro-N-[4-(5-{[3-(4-morpholinyl)propyl]amino}-1,3,4-oxadiazol-2-yl)-3-(trifluoromethyl)phenyl]benzamide | 494 |
| 87 | 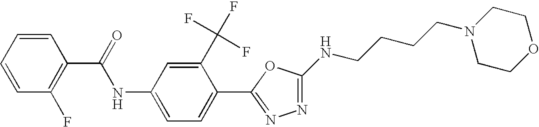 | 2-fluoro-N-[4-(5-{[4-(4-morpholinyl)butyl]amino}-1,3,4-oxadiazol-2-yl)-3-(trifluoromethyl)phenyl]benzamide | 508 |
| 88 | 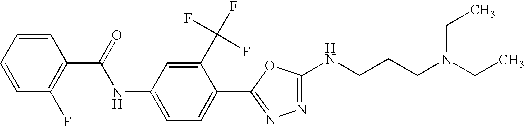 | N-[4-(5-{[3-(diethylamino)propyl]amino}-1,3,4-oxadiazol-2-yl)-3-(trifluoromethyl)phenyl]-2-fluorobenzamide | 480 |
| 89 | 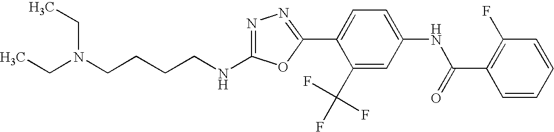 | N-[4-(5-{[4-(diethylamino)butyl]amino}-1,3,4-oxadiazol-2-yl)-3-(trifluoromethyl)phenyl]-2-fluorobenzamide | 494 |
| 90 | 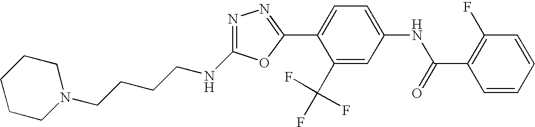 | 2-fluoro-N-[4-(5-{[4-(1-piperidinyl)butyl]amino]-1,3,4-oxadiazol-2-yl)-3-(trifluoromethyl)phenyl]benzamide | 506 |
| 91 | 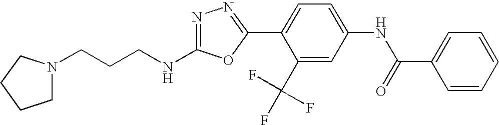 | N-[4-(5-{[3-(1-pyrrolidinyl)propyl]amino}-1,3,4-oxadiazol-2-yl)-3-(trifluoromethyl)phenyl]benzamide | 460 |
| 92 | 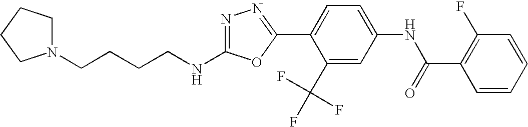 | N-[4-(5-{[4-(1-pyrrolidinyl)butyl]amino}-1,3,4-oxadiazol-2-yl)-3-(trifluoromethyl)phenyl]benzamide | 474 |
| 93 | 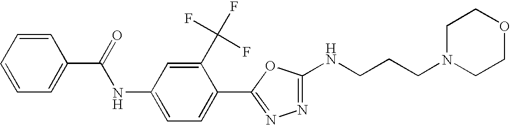 | N-[4-(5-{[3-(4-morpholinyl)propyl]amino}-1,3,4-oxadiazol-2-yl)-3-(trifluoromethyl)phenyl]benzamide | 476 |
| 94 | 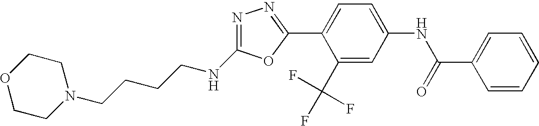 | N-[4-(5-{[4-(4-morpholinyl)butyl]amino}-1,3,4-oxadiazol-2-yl)-3-(trifluoromethyl)phenyl]benzamide | 490 |
| 95 | 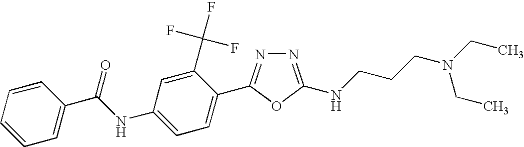 | N-[4-(5-{[3-(diethylamino)propyl]amino}-1,3,4-oxadiazol-2-yl)-3-(trifluoromethyl)phenyl]benzamide | 462 |
| 96 | 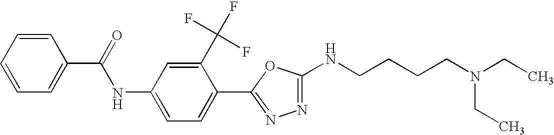 | N-[4-(5-{[4-(diethylamino)butyl]amino}-1,3,4-oxadiazol-2-yl)-3-(trifluoromethyl)phenyl]benzamide | 476 |
| 97 | 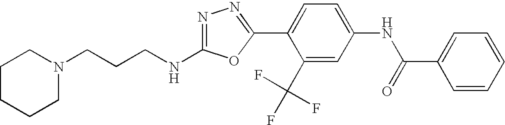 | N-[4-(5-{[3-(1-piperidinyl)propyl]amino}-1,3,4-oxadiazol-2-yl)-3-(trifluoromethyl)phenyl]benzamide | 474 |
| 98 | 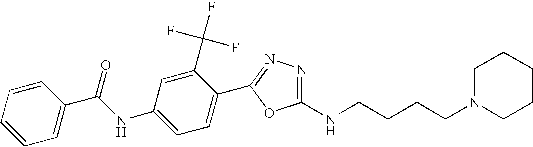 | N-[4-(5-{[4-(1-piperidinyl)butyl]amino}-1,3,4-oxadiazol-2-yl)-3-(trifluoromethyl)phenyl]benzamide | 488 |
| 42 | 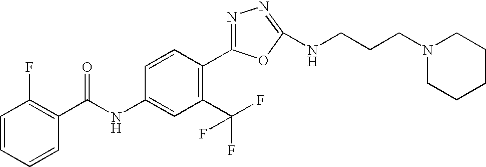 | 2-fluoro-N-[4-(5-{[3-(1-piperidinyl)propyl]amino}-1,3,4-oxadiazol-2-yl)-3-(trifluoromethyl)phenyl]benzamide | 492 |
| 99 | 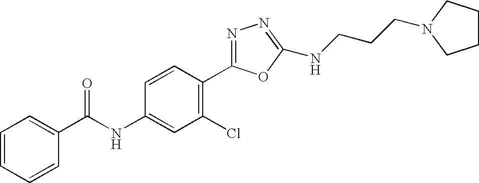 | N-[3-chloro-4-(5-{[4-(1-pyrrolidinyl)propyl]amino}-1,3,4-oxadiazol-2-yl)phenyl]benzamide | 426 |
| 100 | 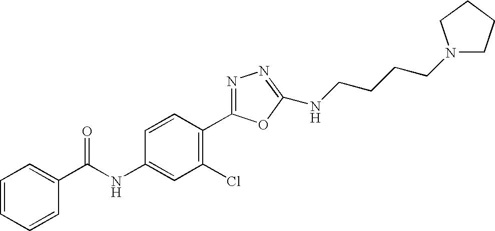 | N-[3-chloro-4-(5-{[4-(1-pyrrolidinyl)butyl]amino}-1,3,4-oxadiazol-2-yl)phenyl]benzamide | 440 |
| 101 | 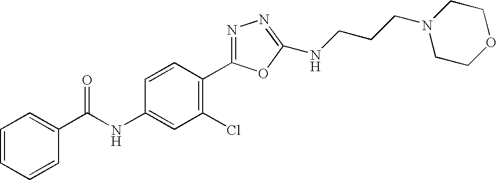 | N-[3-chloro-4-(5-{[3-(4-morpholinyl)propyl]amino}-1,3,4-oxadiazol-2-yl)phenyl]benzamide | 442,444 |
| 102 | 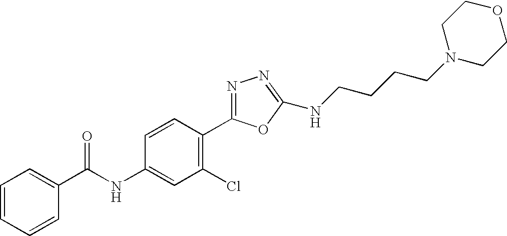 | N-[3-chloro-4-(5-{[4-(4-morpholinyl)butyl]amino}-1,3,4-oxadiazol-2-yl)phenyl]benzamide | 456,458 |
| 103 | 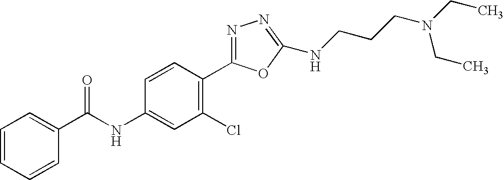 | N-[3-chloro-4-(5-{[3-(diethylamino)propyl]amino}-1,3,4-oxadiazol-2-yl)phenyl]benzamide | 428,430 |
| 104 | 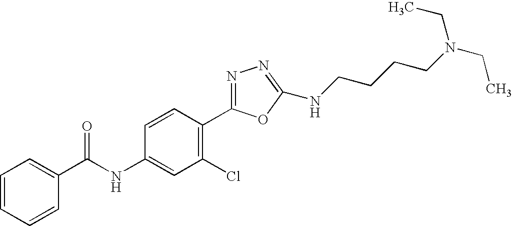 | N-[3-chloro-4-(5-{[4-(diethylamino)butyl]amino}-1,3,4-oxadiazol-2-yl)phenyl]benzamide | 442,444 |
| 105 | 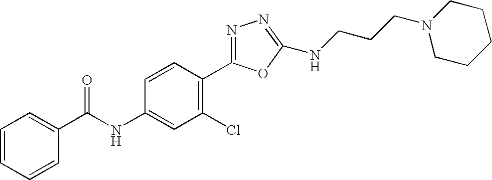 | N-[3-chloro-4-(5-{[3-(1-piperidinyl)propyl]amino}-1,3,4-oxadiazol-2-yl)phenyl]benzamide | 440,442 |
| 106 | 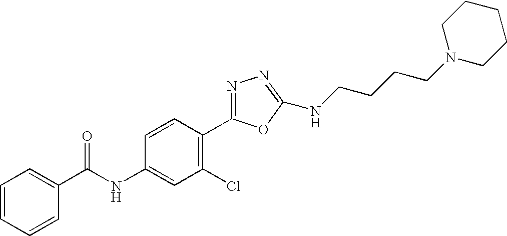 | N-[3-chloro-4-(5-{[4-(1-piperidinyl)butyl]amino}-1,3,4-oxadiazol-2-yl)phenyl]benzamide | 454,456 |
| 107 | 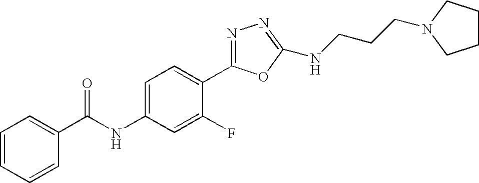 | N-[3-fluoro-4-(5-{[3-(1-pyrrolidinyl)propyl]-amino}-1,3,4-oxadiazol-2-yl)phenyl]benzamide | 410 |
| 108 | 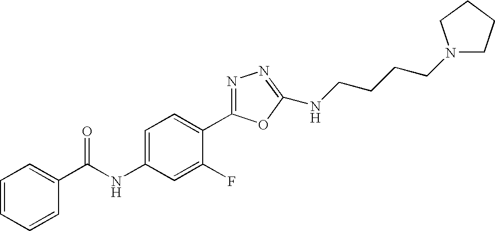 | N-[3-fluoro-4-(5-{[4-(1-pyrrolidinyl)butyl]amino}-1,3,4-oxadiazol-2-yl)phenyl]benzamide | 424 |
| 109 | 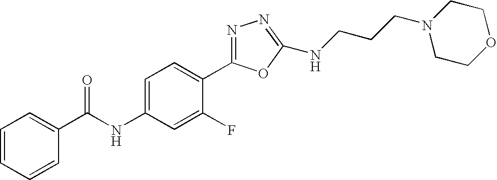 | N-[3-fluoro-4-(5-{[3-(4-morpholinyl)propyl]amino}-1,3,4-oxadiazol-2-yl)phenyl]benzamide | 426 |
| 110 | 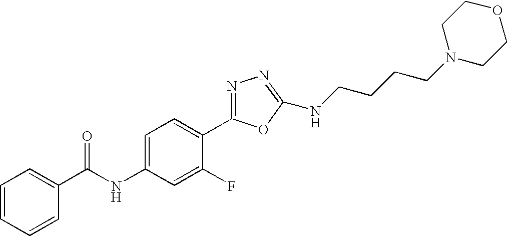 | N-[3-fluoro-4-(5-{[4-(4-morpholinyl)butyl]amino}-1,3,4-oxadiazol-2-yl)phenyl]benzamide | 440 |
| 111 | 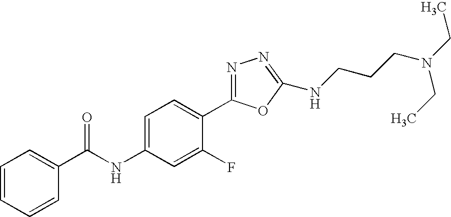 | N-[4-(5-{[3-(diethylamino)propyl]amino}-1,3,4-oxadiazol-2-yl)-3-fluorophenyl]benzamide | 412 |
| 112 | 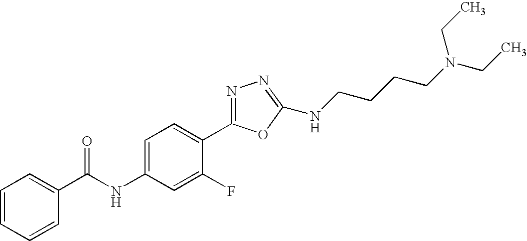 | N-[4-(5-{[4-(diethylamino)butyl]amino}-1,3,4-oxadiazol-2-yl)-3-fluorophenyl]benzamide | 426 |
| 113 | 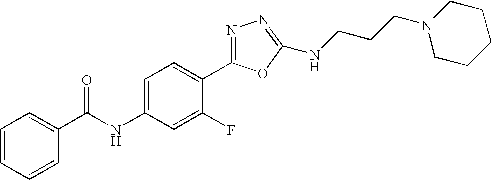 | N-[3-fluoro-4-(5-{[3-(1-piperidinyl)propyl]amino}-1,3,4-oxadiazol-2-yl)phenyl]benzamide | 424 |
| 114 | 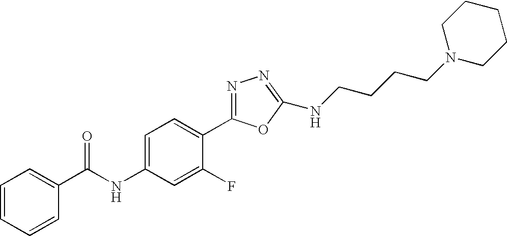 | N-[3-fluoro-4-(5-{[4-(1-piperidinyl)butyl]amino}-1,3,4-oxadiazol-2-yl)phenyl]benzamide | 438 |
| 115 | 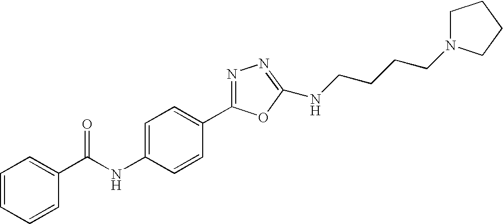 | N-[4-(5-{[4-(1-pyrrolidinyl)butyl]amino}-1,3,4-oxadiazol-2-yl)phenyl]benzamide | 404 |
| 116 | 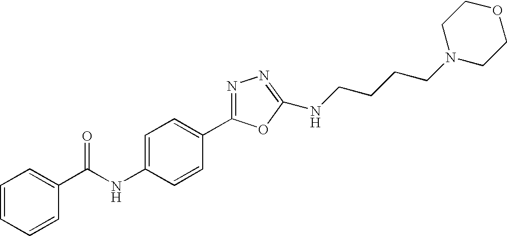 | N-[4-(5-{[4-(4-morpholinyl)butyl]amino}-1,3,4-oxadiazol-2-yl)phenyl]benzamide | 420 |
| 117 | 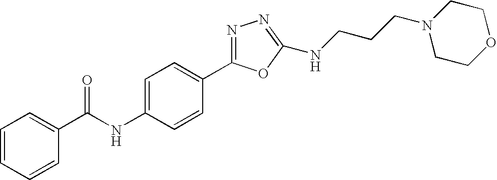 | N-[4-(5-{[3-(4-morpholinyl)propyl]amino}-1,3,4-oxadiazol-2-yl)phenyl]benzamide | 406 |
| 118 | 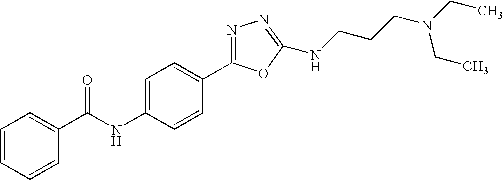 | N-[4-(5-{[3-(diethylamino)propyl]amino}-1,3,4-oxadiazol-2-yl)phenyl]benzamide | 392 |
| 119 | 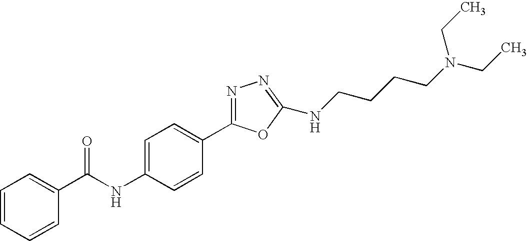 | N-[4-(5-{[4-(diethylamino)butyl]amino}-1,3,4-oxadiazol-2-yl)phenyl]benzamide | 408 |
| 120 | 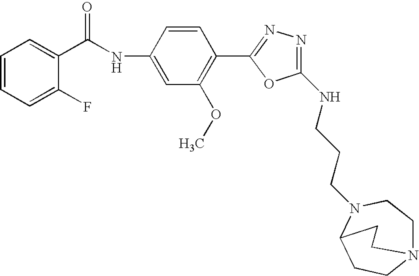 | N-[4-(5-{[3-(1,4-diazabicyclo[3.2.2]non-4-yl)propyl]amino}-1,3,4-oxadiazol-2-yl)-3-(methyloxy)phenyl]-2-fluorobenzamide | 495 |
| 121 | 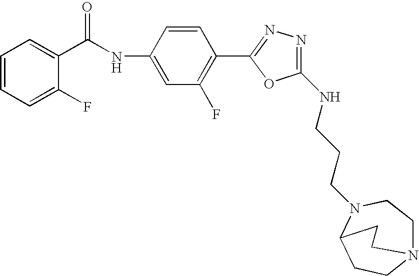 | N-[4-(5-{[3-(1,4-diazabicyclo[3.2.2]non-4-yl)propyl]amino}-1,3,4-oxadiazol-2-yl)-3-fluorophenyl]-2-fluorobenzamide | 483 |
| 122 | 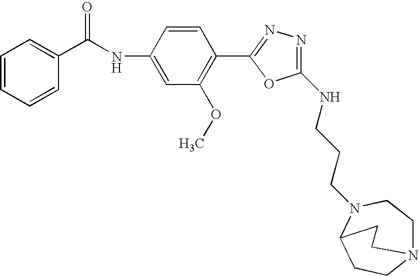 | N-[4-(5-{[3-(1,4-diazabicyclo[3.2.2]non-4-yl)propyl]amino}-1,3,4-oxadiazol-2-yl)-3-(methyloxy)phenyl]benzamide | 477 |
| 123 | 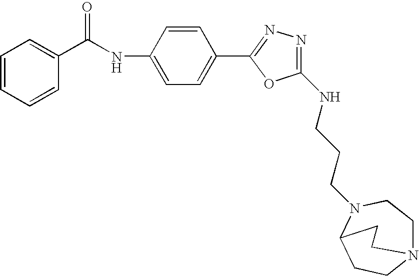 | N-[4-(5-{[3-(1,4-diazabicyclo[3.2.2]non-4-yl)propyl]amino}-1,3,4-oxadiazol-2-yl)phenyl]benzamide | 447 |
| 124 | 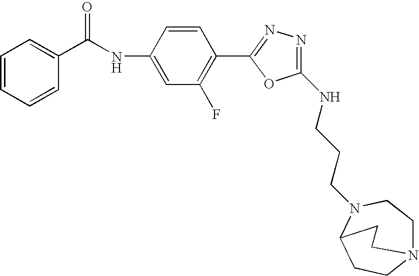 | N-[4-(5-{[3-(1,4-diazabicyclo[3.2.2]non-4-yl)propyl]amino}-1,3,4-oxadiazol-2-yl)-3-fluorophenyl]benzamide | 465 |
| 125 | 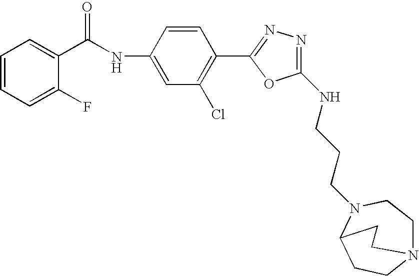 | N-[3-chloro-4-(5-{[3-(1,4-diazabicyclo[3.2.2]non-4-yl)propyl]amino}-1,3,4-oxadiazol-2-yl)phenyl]-2-fluorobenzamide | 499 |
| 126 | 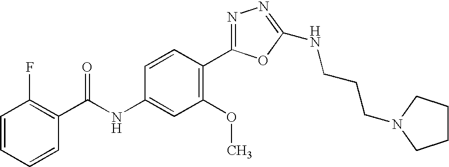 | 2-fluoro-N-[3-(methyloxy)-4-(5-{[3-(1-pyrrolidinyl)propyl]amino}-1,3,4-oxadiazol-2-yl)phenyl]benzamide | 440 |
| 127 | 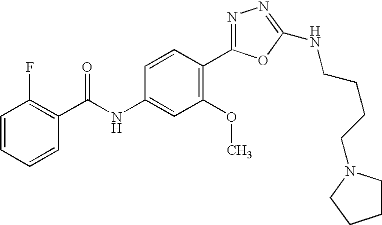 | 2-fluoro-N-[3-(methyloxy)-4-(5-{[4-(1-pyrrolidinyl)butyl]amino}-1,3,4-oxadiazol-2-yl)phenyl]benzamide | 454 |
| 128 | 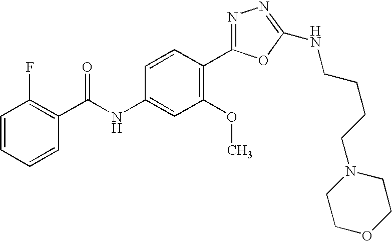 | 2-fluoro-N-[3-(methyloxy)-4-(5-{[4-(4-morpholinyl)butyl]amino}-1,3,4-oxadiazol-2-yl)phenyl]benzamide | 470 |
| 129 | 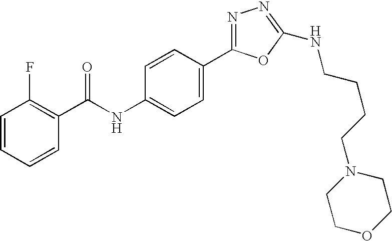 | 2-fluoro-N-[4-(5-{[4-(4-morpholinyl)butyl]amino}-1,3,4-oxadiazol-2-yl)phenyl]benzamide | 440 |
| 130 | 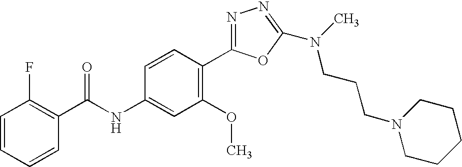 | 2-fluoro-N-[3-(methyloxy)-4-(5-{methyl[3-(1-piperidinyl)propyl]amino}-1,3,4-oxadiazol-2-yl)phenyl]benzamide | 468 |
| 131 | 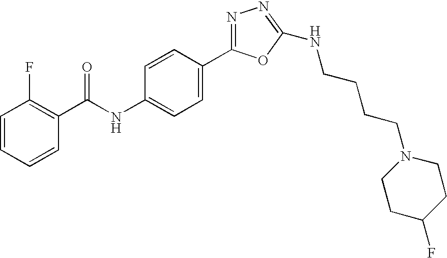 | 2-fluoro-N-[4-(5-{[4-(4-fluoro-1-piperidinyl)butyl]amino}-1,3,4-oxadiazol-2-yl)phenyl]benzamide | 456 |
| 132 | 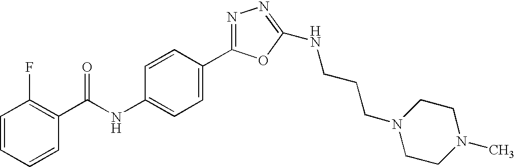 | 2-fluoro-N-[4-(5-{[3-(4-methyl-1-piperazinyl)propyl]amino}-1,3,4-oxadiazol-2-yl)phenyl]benzamide | 439 |
| 133 | 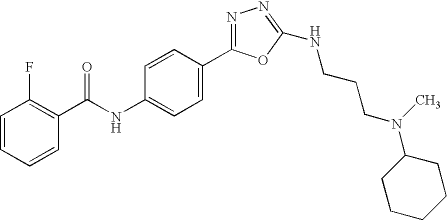 | N-{4-[5-({3-[cyclohexyl(methyl)amino]propyl}amino)-1,3,4-oxadiazol-2-yl]phenyl}-2-fluorobenzamide | 452 |
| Ex 43 | 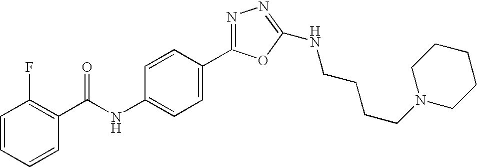 | 2-fluoro-N-[4-(5-{[4-(1-piperidinyl)butyl]amino}-1,3,4-oxadiazol-2-yl)phenyl]benzamide | 438 |
| FreeBase ofEx 21 | 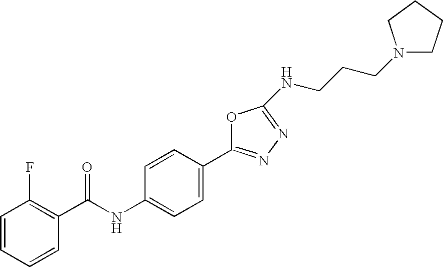 | 2-fluoro-N-[4-(5-{[3-(1-pyrrolidinyl)propyl]amino}-1,3,4-oxadiazol-2-yl)phenyl]benzamide | 410 |
| 134 | 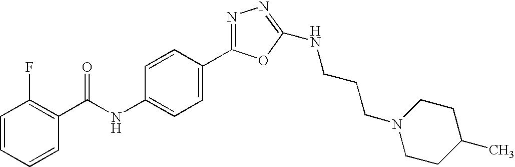 | 2-fluoro-N-[4-(5-{[3-(4-methyl-1-piperidinyl)propyl]amino}-1,3,4-oxadiazol-2-yl)phenyl]benzamide | 438 |
| 135 | 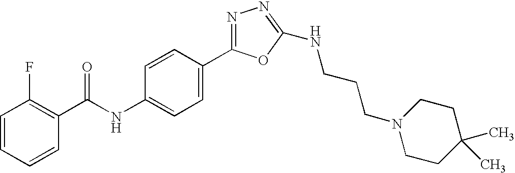 | N-[4-(5-{[3-(4,4-dimethyl-1-piperidinyl)propyl]amino}-1,3,4-oxadiazol-2-yl)phenyl]-2-fluorobenzamide | 452 |
| 136 | 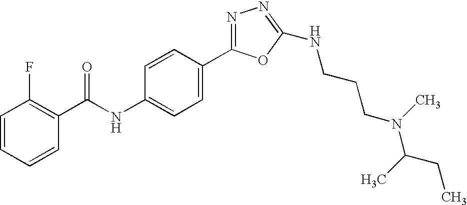 | 2-fluoro-N-{4-[5-({3-[methyl(1-methylpropyl)amino]propyl}amino)-1,3,4-oxadiazol-2-yl]phenyl}benzamide | 426 |
| 137 | 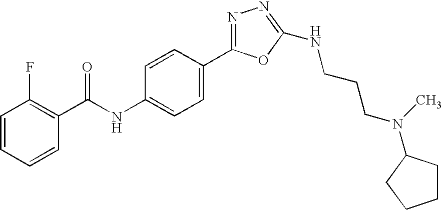 | N-{4-[5-({3-[cyclopentyl(methyl)amino]propyl}amino)-1,3,4-oxadiazol-2-yl]phenyl}-2-fluorobenzamide | 438 |
| 138 | 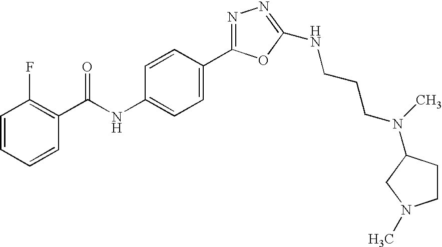 | 2-fluoro-N-{4-[5-({3-[methyl(1-methyl-3-pyrrolidinyl)amino]propyl}amino)-1,3,4-oxadiazol-2-yl]phenyl}benzamide | 453 |
| 139 | 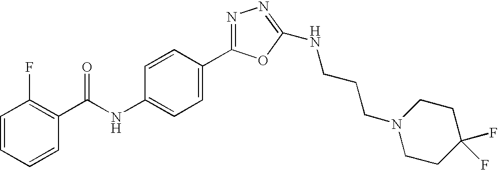 | N-[4-(5-{[3-(4,4-difluoro-1-piperidinyl)propyl]amino}-1,3,4-oxadiazol-2-yl)phenyl]-2-fluorobenzamide | 460 |
| 140 | 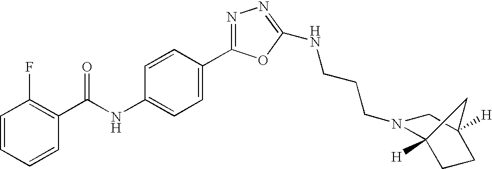 | N-{4-[5-({3-[(1S,4R)-2-azabicyclo[2.2.1]hept-2-yl]propyl}amino)-1,3,4-oxadiazol-2-yl]phenyl}-2-fluorobenzamide | 436 |
| 141 | 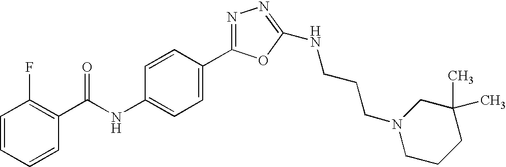 | N-[4-(5-{[3-(3,3-dimethyl-1-piperidinyl)propyl]amino}-1,3,4-oxadiazol-2-yl)phenyl]-2-fluorobenzamide | 452 |
| 142 | 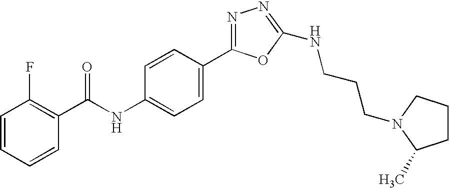 | 2-fluoro-N-{4-[5-({3-[(2S)-2-methyl-1-pyrrolidinyl]propyl}amino)-1,3,4-oxadiazol-2-yl]phenyl}benzamide | 424 |
| 143 | 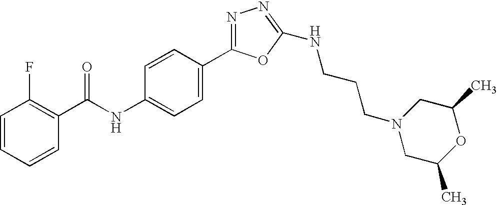 | N-{4-[5-({3-[(2R,6S)-2,6-dimethyl-4-morpholinyl]propyl}amino)-1,3,4-oxadiazol-2-yl]phenyl}-2-fluorobenzamide | 454 |
| 144 | 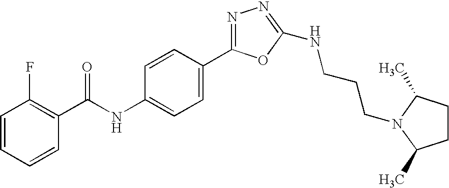 | N-{4-[5-({3-[(2R,5R)-2,5-dimethyl-1-pyrrolidinyl]propyl}amino)-1,3,4-oxadiazol-2-yl]phenyl}-2-fluorobenzamide | 438 |
| Ex 44 | 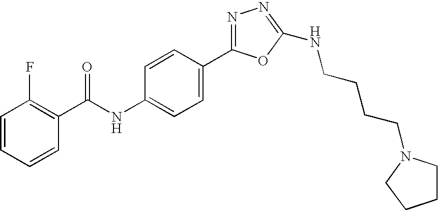 | 2-fluoro-N-[4-(5-{[4-(1-pyrrolidinyl)butyl]amino}-1,3,4-oxadiazol-2-yl)phenyl]benzamide | 424 |
| 145 | 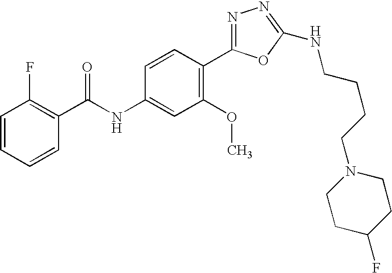 | 2-fluoro-N-[4-(5-{[4-(4-fluoro-1-piperidinyl)butyl]amino}-1,3,4-oxadiazol-2-yl)-3-(methyloxy)phenyl]benzamide | 486 |
| 146 | 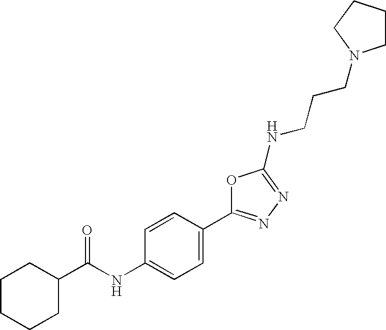 | N-[4-(5-{[3-(1-pyrrolidinyl)propyl]amino}-1,3,4-oxadiazol-2-yl)phenyl]cyclohexanecarboxamide | 398 |
| 147 | 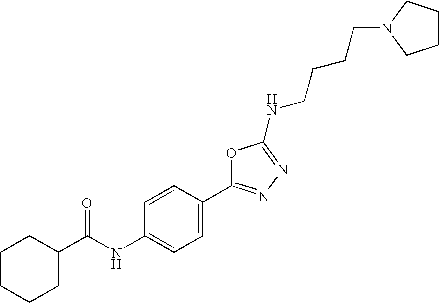 | N-[4-(5-{[4-(1-pyrrolidinyl)butyl]amino}-1,3,4-oxadiazol-2-yl)phenyl]cyclohexanecarboxamide | 412 |
| 148 | 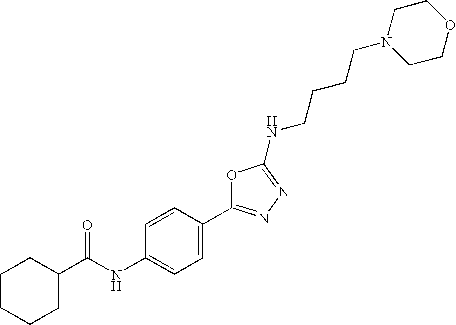 | N-[4-(5-{[4-(4-morpholinyl)butyl]amino}-1,3,4-oxadiazol-2-yl)phenyl]cyclohexanecarboxamide | 428 |
| 149 | 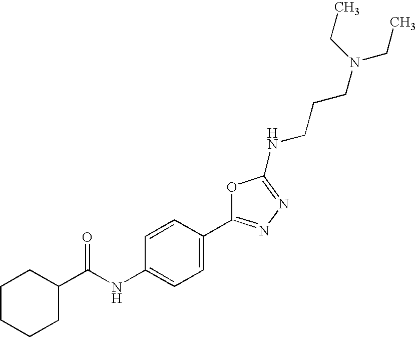 | N-[4-(5-{[3-(diethylamino)propyl]amino}-1,3,4-oxadiazol-2-yl)phenyl]cyclohexanecarboxamide | 400 |
| 150 | 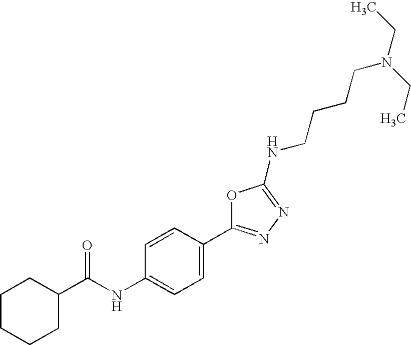 | N-[4-(5-{[4-(diethylamino)butyl]amino}-1,3,4-oxadiazol-2-yl)phenyl]cyclohexanecarboxamide | 414 |
| 151 | 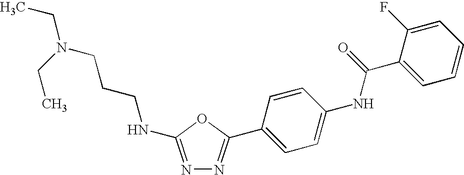 | N-[4-(5-{[3-(diethylamino)propyl]amino}-1,3,4-oxadiazol-2-yl)phenyl]-2-fluorobenzamide | 412 |
| 152 | 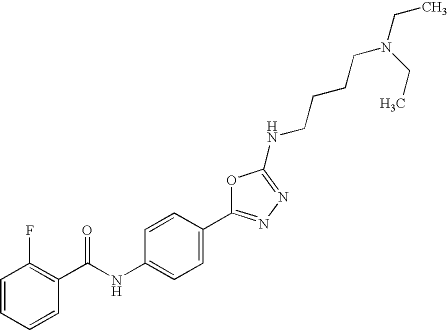 | N-[4-(5-{[4-(diethylamino)butyl]amino}-1,3,4-oxadiazol-2-yl)phenyl]-2-fluorobenzamide | 426 |
| 153 | 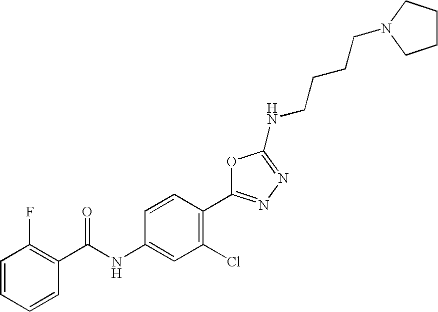 | N-[3-chloro-4-(5-{[4-(1-pyrrolidinyl)butyl]amino}-1,3,4-oxadiazol-2-yl)phenyl]-2-fluorobenzamide | 458,460 |
| 154 | 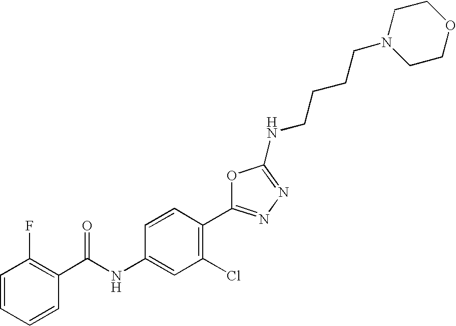 | N-[3-chloro-4-(5-{[4-(4-morpholinyl)butyl]amino}-1,3,4-oxadiazol-2-yl)phenyl]-2-fluorobenzamide | 474,476 |
| 155 | 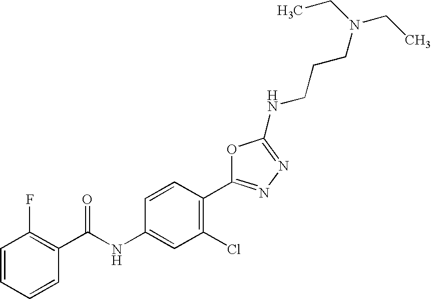 | N-[3-chloro-4-(5-{[3-(diethylamino)propyl]amino}-1,3,4-oxadiazol-2-yl)phenyl]-2-fluorobenzamide | 446;448 |
| 156 | 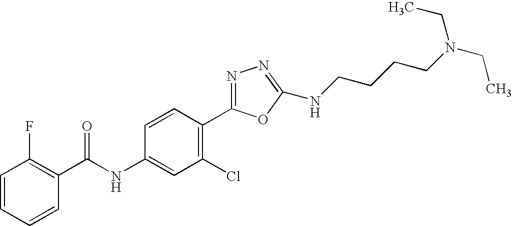 | N-[3-chloro-4-(5-{[4-(diethylamino)butyl]amino}-1,3,4-oxadiazol-2-yl)phenyl]-2-fluorobenzamide | 460;462 |
| 157 | 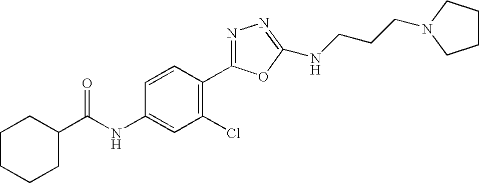 | N-[3-chloro-4-(5-{[3-(1-pyrrolidinyl)propyl]amino}-1,3,4-oxadiazol-2-yl)phenyl]cyclohexanecarboxamide | 432;434 |
| 158 | 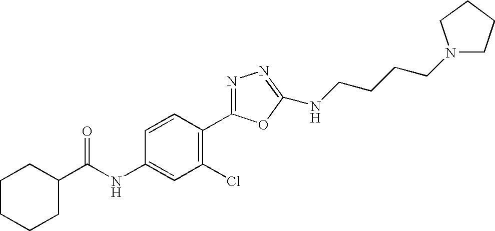 | N-[3-chloro-4-(5-{[4-(1-pyrrolidinyl)butyl]amino}-1,3,4-oxadiazol-2-yl)phenyl]cyclohexanecarboxamide | 446;448 |
| 159 | 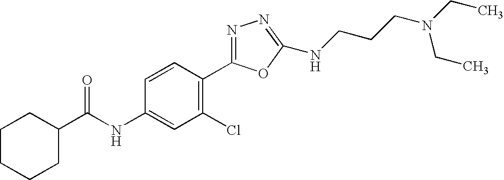 | N-[3-chloro-4-(5-{[3-(diethylamino)propyl]amino}-1,3,4-oxadiazol-2-yl)phenyl]cyclohexanecarboxamide | 434;436 |
| 160 | 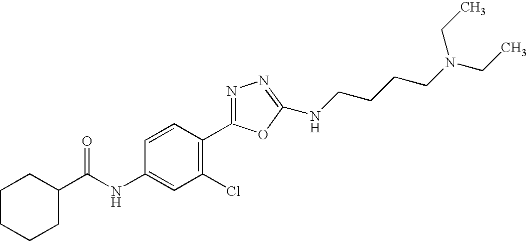 | N-[3-chloro-4-(5-{[4-(diethylamino)butyl]amino}-1,3,4-oxadiazol-2-yl)phenyl]cyclohexanecarboxamide | 448;450 |
| 161 | 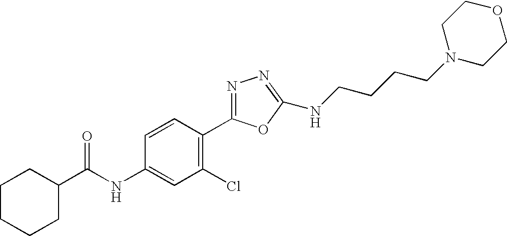 | N-[3-chloro-4-(5-{[4-(4-morpholinyl)butyl]amino}-1,3,4-oxadiazol-2-yl)phenyl]cyclohexanecarboxamide | 462;464 |
| 162 | 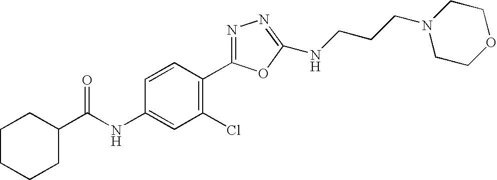 | N-[3-chloro-4-(5-{[3-(4-morpholinyl)propyl]amino}-1,3,4-oxadiazol-2-yl)phenyl]cyclohexanecarboxamide | 448;451 |
| 163 | 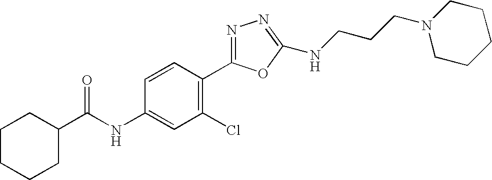 | N-[3-chloro-4-(5-{[3-(1-piperidinyl)propyl]amino}-1,3,4-oxadiazol-2-yl)phenyl]cyclohexanecarboxamide | 446;448 |
| 164 | 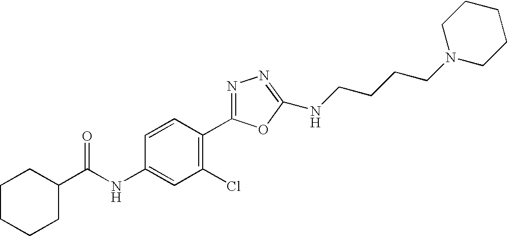 | N-[3-chloro-4-(5-{[4-(1-piperidinyl)butyl]amino}-1,3,4-oxadiazol-2-yl)phenyl]cyclohexanecarboxamide | 460;462 |
| 165 | 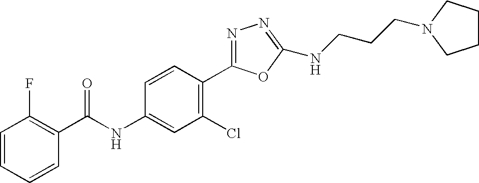 | N-[3-chloro-4-(5-{[3-(1-pyrrolidinyl)propyl]amino}-1,3,4-oxadiazol-2-yl)phenyl]-2-fluorobenzamide | 444;446 |
| 166 | 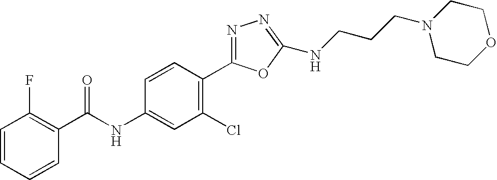 | N-[3-chloro-4-(5-{[3-(4-morpholinyl)propyl]amino}-1,3,4-oxadiazol-2-yl)phenyl]-2-fluorobenzamide | 460;462 |
| 167 | 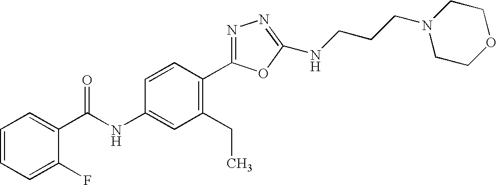 | N-[3-ethyl-4-(5-{[3-(4-morpholinyl)propyl]amino}-1,3,4-oxadiazol-2-yl)phenyl]-2-fluorobenzamide | 454 |
| 168 | 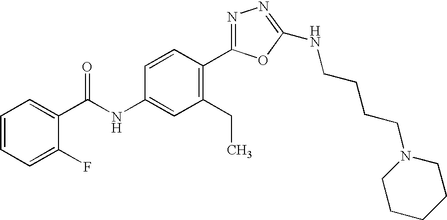 | N-[3-ethyl-4-(5-{[4-(1-piperidinyl)butyl]amino}-1,3,4-oxadiazol-2-yl)phenyl]-2-fluorobenzamide | 466 |
| 169 | 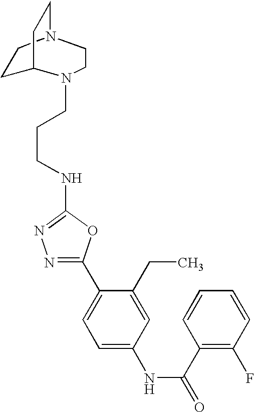 | N-[4-(5-{[3-(1,4-diazabicyclo[3.2.2]non-4-yl)propyl]amino}-1,3,4-oxadiazol-2-yl)-3-ethylphenyl]-2-fluorobenzamide | 493 |
| 170 | 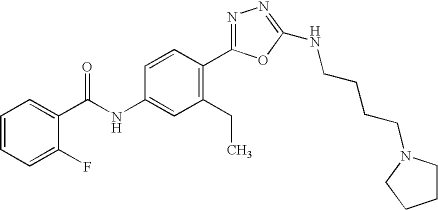 | N-[3-ethyl-4-(5-{[4-(1-pyrrolidinyl)butyl]amino}-1,3,4-oxadiazol-2-yl)phenyl]-2-fluorobenzamide | 452 |
| 171 | 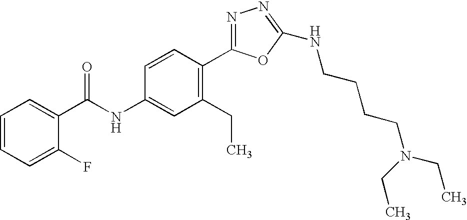 | N-[4-(5-{[4-(diethylamino)butyl]amino}-1,3,4-oxadiazol-2-yl)-3-ethylphenyl]-2-fluorobenzamide | 454 |
| 172 | 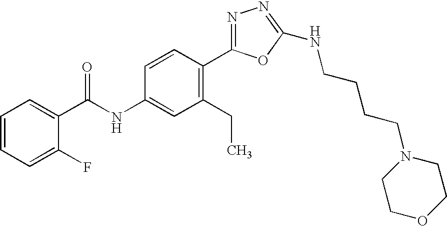 | N-[3-ethyl-4-(5-{[4-(4-morpholinyl)butyl]amino}-1,3,4-oxadiazol-2-yl)phenyl]-2-fluorobenzamide | 468 |
| 173 | 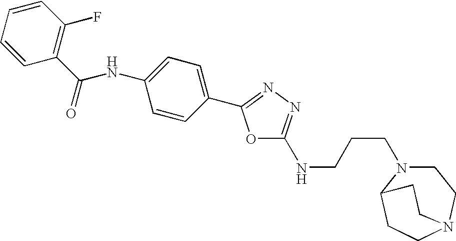 | N-[4-(5-{[3-(1,4-diazabicyclo[3.2.2]non-4-yl)propyl]amino}-1,3,4-oxadiazol-2-yl)phenyl]-2-fluorobenzamide | 465 |
| 174 | 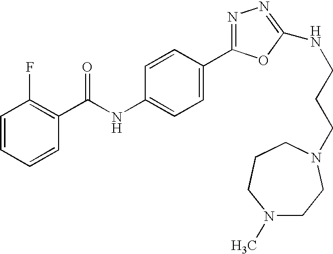 | 2-fluoro-N-[4-(5-{[3-(4-methylhexahydro-1H-1,4-diazepin-1-yl)propyl]amino}-1,3,4-oxadiazol-2-yl)phenyl]benzamide | 453 |
| 175 | 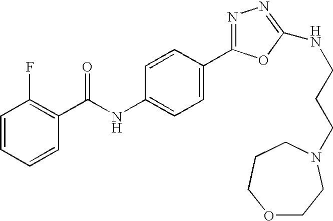 | 2-fluoro-N-[4-(5-{[3-(tetrahydro-1,4-oxazepin-4(5H)-yl)propyl]amino}-1,3,4-oxadiazol-2-yl)phenyl]benzamide | 440 |
| 176 | 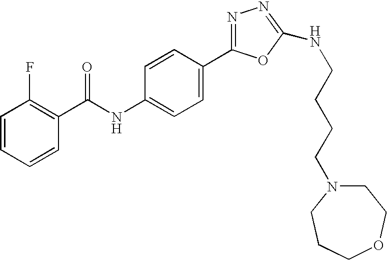 | 2-fluoro-N-[4-(5-{[4-(tetrahydro-1,4-oxazepin-4(5H)-yl)butyl]amino}-1,3,4-oxadiazol-2-yl)phenyl]benzamide | 454 |

Compounds of Examples 177 to 192 were prepared in a similar fashion to Example 6, except that the treatment with HCl was omitted and the Examples were isolated as the free base.

Variations to the procedure used in Example 6 include, but are not limited to, the use of 4M HCl in dioxane instead of trifluoroacetic acid in DCM, the reaction time, and the purification method used.

TABLE 22
Examples 177 to 190

| Example | | | |
|---|---|---|---|
| number | Structure | Name | [M + H]+ |
| 177 | 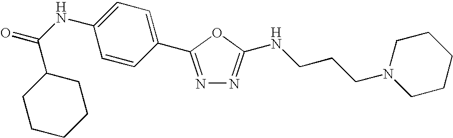 | N-[4-(5-{[3-(1-piperidinyl)propyl]amino}-1,3,4-oxadiazol-2-yl)phenyl]cyclohexane-carboxamide | 412 |
| 178 | 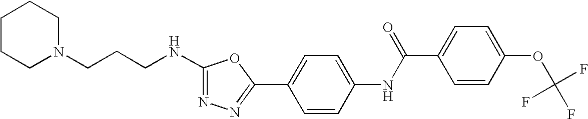 | N-[4-(5-{[3-(1-piperidinyl)propyl]amino}-1,3,4-oxadiazol-2-yl)phenyl]-4-[(trifluoromethyl)oxy]benzamide | 490 |
| 179 | 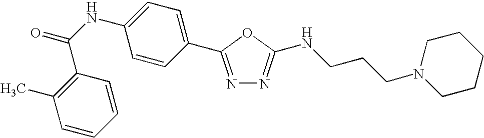 | 2-methyl-N-[4-(5-{[3-(1-piperidinyl)propyl]amino}-1,3,4-oxadiazol-2-yl)phenyl]benzamide | 420 |
| 180 | 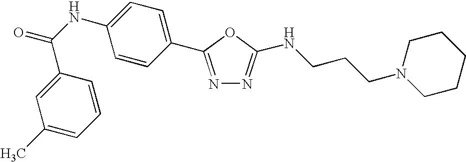 | 3-methyl-N-[4-(5-{[3-(1-piperidinyl)propyl]amino}-1,3,4-oxadiazol-2-yl)phenyl]benzamide | 420 |
| 181 | 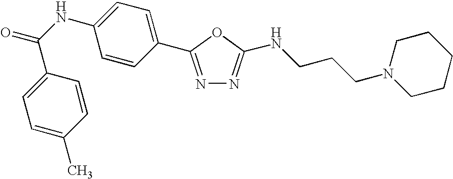 | 4-methyl-N-[4-(5-{[3-(1-piperidinyl)propyl]amino}-1,3,4-oxadiazol-2-yl)phenyl]benzamide | 420 |
| 182 | 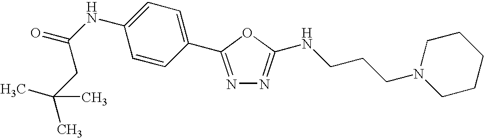 | 3,3-dimethyl-N-[4-(5-{[3-(1-piperidinyl)propyl]amino}-1,3,4-oxadiazol-2-yl)phenyl]butanamide | 400 |
| Example6 FreeBase | 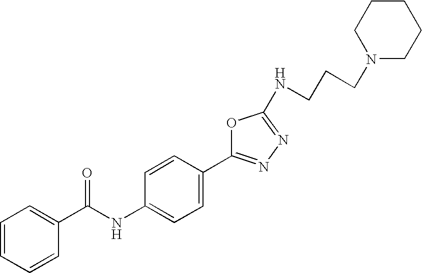 | N-[4-(5-{[3-(1-piperidinyl)propyl]amino}-1,3,4-oxadiazol-2-yl)phenyl]benzamide | 406 |
| 183 | 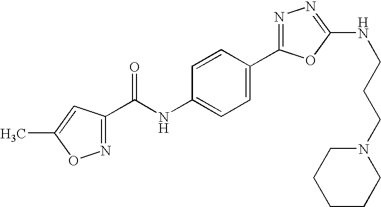 | 5-methyl-N-[4-(5-{[3-(1-piperidinyl)propyl]amino}-1,3,4-oxadiazol-2-yl)phenyl]-3-isoxazolecarboxamide | 411 |
| 184 | 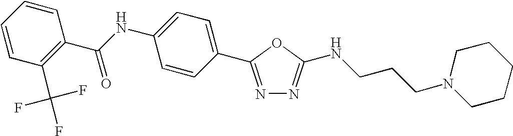 | N-[4-(5-{[3-(1-piperidinyl)propyl]amino}-1,3,4-oxadiazol-2-yl)phenyl]-2-(trifluoromethyl)benzamide | 474 |
| 185 | 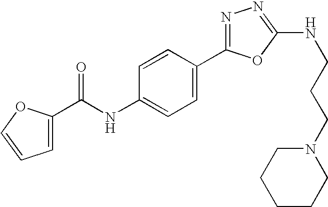 | N-[4-(5-{[3-(1-piperidinyl)propyl]amino}-1,3,4-oxadiazol-2-yl)phenyl]-2-furancarboxamide | 396 |
| 186 | 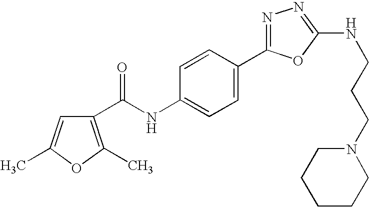 | 2,5-dimethyl-N-[4-(5-{[3-(1-piperidinyl)propyl]amino}-1,3,4-oxadiazol-2-yl)phenyl]-3-furancarboxamide | 424 |
| 187 | 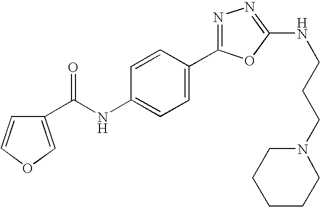 | N-[4-(5-{[3-(1-piperidinyl)propyl]amino}-1,3,4-oxadiazol-2-yl)phenyl]-3-furancarboxamide | 396 |
| 188 | 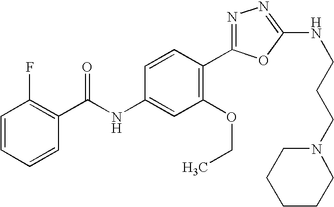 | N-[3-(ethyloxy)-4-(5-{[3-(1-piperidinyl)propyl]amino}-1,3,4-oxadiazol-2-yl)phenyl]-2-fluorobenzamide | 468 |
| 189 | 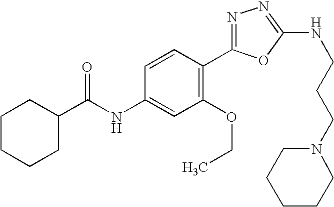 | N-[3-(ethyloxy)-4-(5-{[3-(1-piperidinyl)propyl]amino}-1,3,4-oxadiazol-2-yl)phenyl]cyclohexane-carboxamide | 456 |
| 190 | 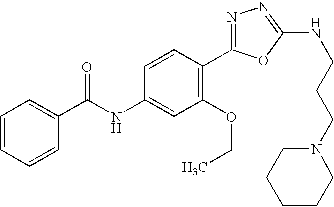 | N-[3-(ethyloxy)-4-(5-{[3-(1-piperidinyl)propyl]amino}-1,3,4-oxadiazol-2-yl)phenyl]benzamide | 450 |

### Example 191

### N-[4-(5-{[3-(1-piperidinyl)propyl]amino}-1,3,4-oxadiazol-2-yl)phenyl]cycloheptanecarboxamide

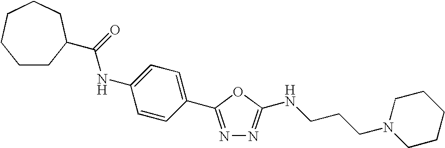

Thionyl chloride (417 mg, 3.5 mmol) was added to a solution of cycloheptanecarboxylic acid (50 mg, 0.35 mmol) in dichloromethane (1 mL) and the solution heated to 40° C. for 1.5 h. The mixture was concentrated in vacuo and the residue re-dissolved in dichloromethane (2 mL) and added to a solution of 1,1-dimethylethyl [5-(4-aminophenyl)-1,3,4-oxadiazol-2-yl][3-(1-piperidinyl)propyl]carbamate (2 mL) followed by triethylamine (71 mg, 0.7 mmol). The reaction was stirred at room temperature overnight, after which time only partial reaction was observed. Further cycloheptanecarbonyl chloride (1.1 mmol) was synthesised as above and added to the reaction mixture along with further triethylamine (213 mg, 2.1 mmol) and the reaction stirred for a further 24 h. The solvent was removed in vacuo and the residue dissolved in 4M HCl in dioxan (2 mL) and stirred at room temperature overnight. The solvent was removed in vacuo and the crude product partially purified by column chromatography [silica gel, 2M ammonia in methanol:dichloromethane (5-10%)]. The product was further purified by MDAP and the product applied on SCX cartridge, washed with methanol and eluted with 2M ammonia in methanol before removal of solvent in vacuo to give the title compound (85 mg). LCMS (ES+ve): [M+H]⁺ at m/z 426 (C₂₄H₃₅N₅O₂ requires [M+H]⁺ at m/z 426).

In the preparation of the following compounds Example 192 and 193 in Table 23, the acid chloride reagent used to prepare the Boc protected material was synthesised and then reacted directly with the appropriate aniline, in a similar manner to Example 191.

TABLE 23

| Example | | | |
|---|---|---|---|
| Number | Structure | Name | [M + H]⁺ |
| 192 | 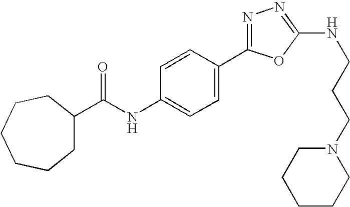 | N-[4-(5-{[3-(1-piperidinyl)propyl]amino}-1,3,4-oxadiazol-2-yl)phenyl]cycloheptane-carboxamide | 426 |
| 193 | 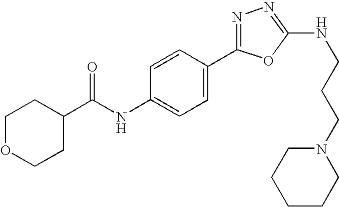 | N-[4-(5-{[3-(1-piperidinyl)propyl]amino}-1,3,4-oxadiazol-2-yl)phenyl]tetrahydro-2H-pyran-4-carboxamide | 414 |

### Example 194

### 2,6-Difluoro-N-[4-(5-{[3-(4-morpholinyl)propyl]amino}-1,3,4-oxadiazol-2-yl)phenyl]benzamide

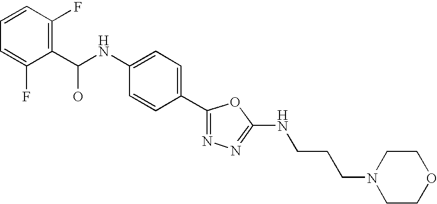

To a suspension of 2,6-difluoro-N-(4-{[2-({[3-(1-piperidinyl)propyl]amino}carbonothioyl)hydrazino]carbonyl}phenyl)benzamide (250 mg, 0.52 mmol, may be prepared as in Description 36) in industrial methylated spirits (5 mL) at 0° C. was added 2M aqueous sodium hydroxide solution until pH 9. Aqueous 5% KI₃ solution (ca 7 mL) was then added dropwise until the orange colour persisted. LC/MS showed 82% required product and 12% remaining starting material so a further portion (2 mL) of 5% aqueous KI₃ solution was added and the reaction mixture stirred for 1 hour. LC/MS then showed 93% required product. The reaction mixture was concentrated and the residue was triturated with dichloromethane containing a few drops of methanol. The organics were concentrated and the residue was columned on silica gel with eluent increasing in polarity from dichloromethane to dichloromethane containing 7% methanolic ammonia to give the title compound (182.5 mg) as a yellow oil. LC/MS (ES+ve): [M+H]⁺ 444 at m/z (C₂₂H₂₃F₂N₅O₂ requires [M+H]⁺ at m/z 444).

The following compounds were prepared in a similar manner:

TABLE 24
Examples 195-205

| Example | | | |
|---|---|---|---|
| no. | Structure | Name | [M + H]⁺ |
| 195 | 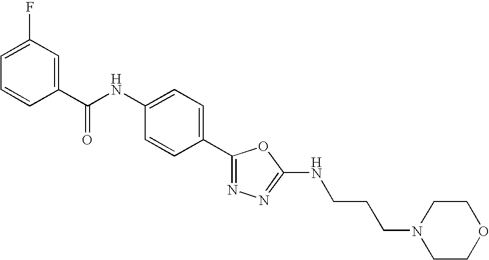 | 3-Fluoro-N-[4-(5-{[3-(4-morpholinyl)propyl]amino}-1,3,4-oxadiazol-2-yl)phenyl]benzamide | 426 |
| 196 | 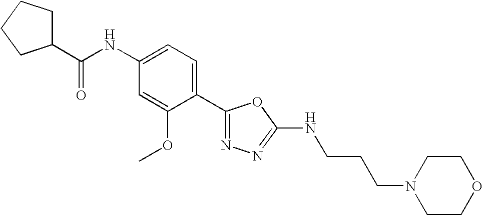 | N-[3-(Methyloxy)-4-(5-{[3-(4-morpholinyl)propyl]amino}-1,3,4-oxadiazol-2-yl)phenyl]cyclopentane-carboxamide | 430 |
| 197 | 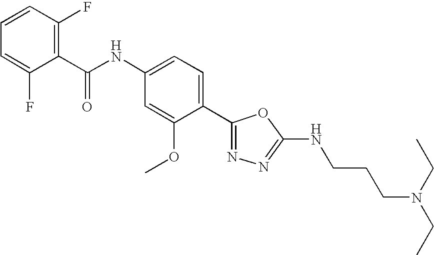 | N-[4-(5-{[3-(Diethylamino)propyl]amino}-1,3,4-oxadiazol-2-yl)-3-(methyloxy)phenyl]-2,6-difluorobenzamide | 460 |
| 198 | 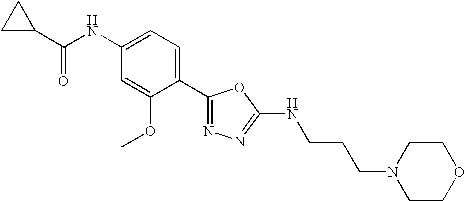 | N-[3-(Methyloxy)-4-(5-{[3-(4-morpholinyl)propyl]amino}-1,3,4-oxadiazol-2-yl)phenyl]cyclopropane-carboxamide | 402 |
| 199 | 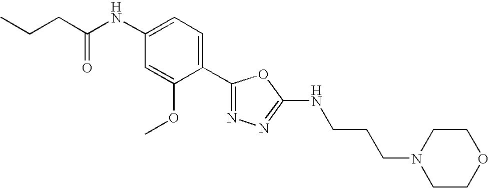 | N-[3-(Methyloxy)-4-(5-{[3-(4-morpholinyl)propyl]amino}-1,3,4-oxadiazol-2-yl)phenyl]butanamide | 404 |
| 200 | 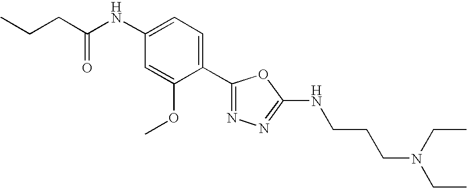 | N-[4-(5-{[3-(Diethylamino)propyl]amino}-1,3,4-oxadiazol-2-yl)-3-(methyloxy)phenyl]butanamide | 390 |
| 201 | 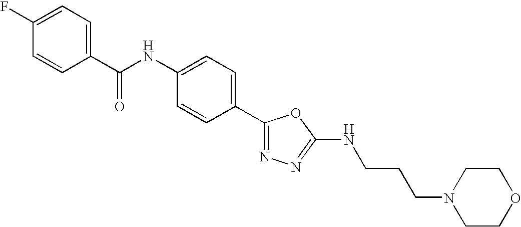 | 4-Fluoro-N-[4-(5-{[3-(4-morpholinyl)propyl]amino}-1,3,4-oxadiazol-2-yl)phenyl]benzamide | 426 |
| 202 | 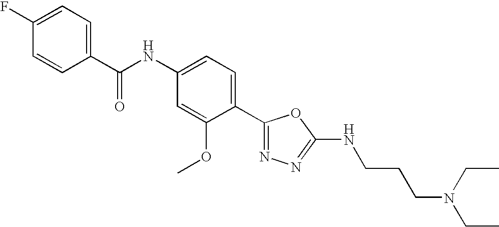 | N-[4-(5-{[3-(Diethylamino)propyl]amino}-1,3,4-oxadiazol-2-yl)-3-(methyloxy)phenyl]-4-fluorobenzamide | 442 |
| 203 | 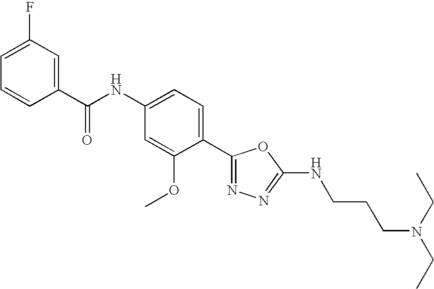 | N-[4-(5-{[3-(Diethylamino)propyl]amino}-1,3,4-oxadiazol-2-yl)-3-(methyloxy)phenyl]-3-fluorobenzamide | 442 |
| 204 | 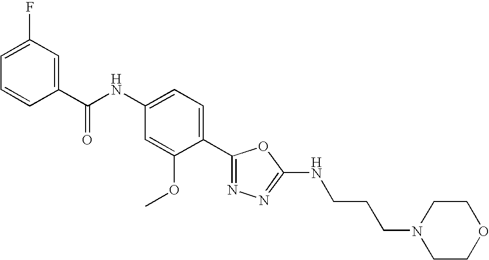 | 3-Fluoro-N-[3-(methyloxy)-4-(5-{[3-(4-morpholinyl)propyl]amino}-1,3,4-oxadiazol-2-yl)phenyl]benzamide | 456 |
| 205 | 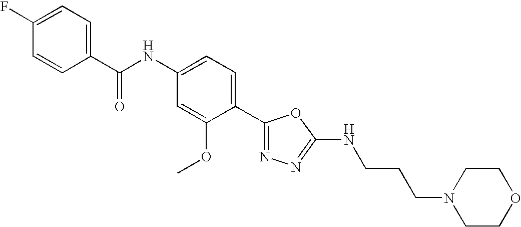 | 4-Fluoro-N-[3-(methyloxy)-4-(5-{[3-(4-morpholinyl)propyl]amino}-1,3,4-oxadiazol-2-yl)phenyl]benzamide | 456 |

Example 194, 195 and 197 to 205 were also prepared as hydrochloride salts shown below as Examples 194 (HCl), 195 (HCl) and 197 (HCl) to 205 (HCl).

### Example 194 HCl

### 2,6-Difluoro-N-[4-(5-{[3-(4-morpholinyl)propyl]amino}-1,3,4-oxadiazol-2-yl)phenyl]benzamide Hydrochloride

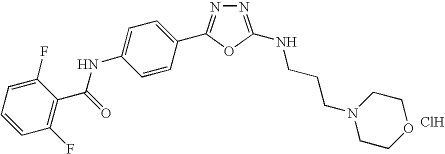

To a solution of 2,6-difluoro-N-[4-(5-{[3-(4-morpholinyl)propyl]amino}-1,3,4-oxadiazol-2-yl)phenyl]benzamide (180 mg, 0.41 mmol) in dioxan (3 mL) and methanol (2 mL) was added 4M HCl in dioxan (2 mL). The reaction mixture was stirred overnight and then concentrated. The residue was recrystallised from methanol/ethyl acetate to give the title compound (174 mg) as a yellow solid. LC/MS (ES+ve): [M+H]⁺ m/z 444 (C₂₂H₂₃F₂N₅O₃ requires [M+H]⁺ at m/z 444).

The following compounds were prepared in an analogous manner by treatment of the appropriate free base with a solution of HCl in dioxan (the time standing in HCl in dioxan may vary from 2 hours to overnight).

TABLE 25
Examples 195 (HCl), 197 (HCl) to 205 (HCl)

| Example | | | |
|---|---|---|---|
| no. | Structure | Name | [M + H]⁺ |
| 195(HCl) | 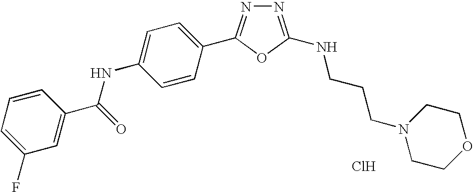 | 3-fluoro-N-[4-(5-{[3-(4-morpholinyl)propyl]amino}-1,3,4-oxadiazol-2-yl)phenyl]benzamidehydrochloride | 426 |
| 197(HCl) | 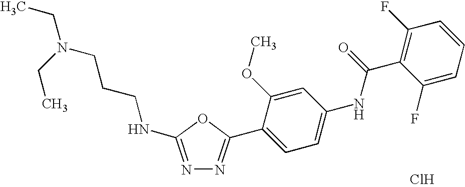 | N-[4-(5-{[3-(diethylamino)propyl]amino}-1,3,4-oxadiazol-2-yl)-3-(methyloxy)phenyl]-2,6-difluorobenzamide hydrochloride | 460 |
| 198(HCl) | 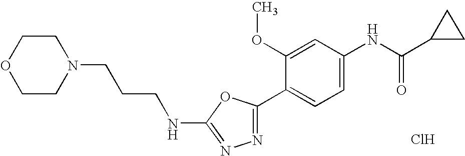 | N-[3-(methyloxy)-4-(5-{[3-(4-morpholinyl)propyl]amino}-1,3,4-oxadiazol-2-yl)phenyl]cyclopropanecarboxamidehydrochloride | 402 |
| 199(HCl) | 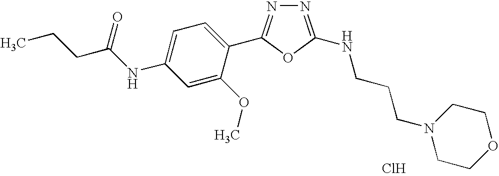 | N-[3-(methyloxy)-4-(5-{[3-(4-morpholinyl)propyl]amino}-1,3,4-oxadiazol-2-yl)phenyl]butanamidehydrochloride | 404 |
| 200(HCl) | 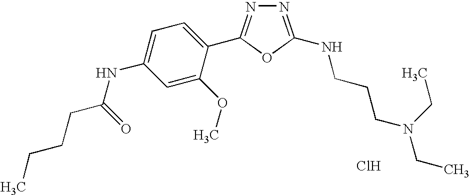 | N-[4-(5-{[3-(diethylamino)propyl]amino}-1,3,4-oxadiazol-2-yl)-3-(methyloxy)phenyl]butanamidehydrochloride | 390 |
| 201(HCl) | 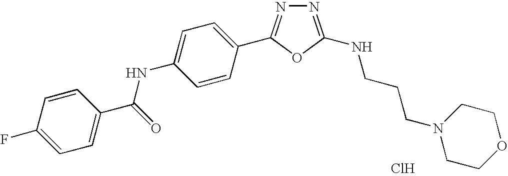 | 4-fluoro-N-[4-(5-{[3-(4-morpholinyl)propyl]amino}-1,3,4-oxadiazol-2-yl)phenyl]benzamidehydrochloride | 426 |
| 202(HCl) | 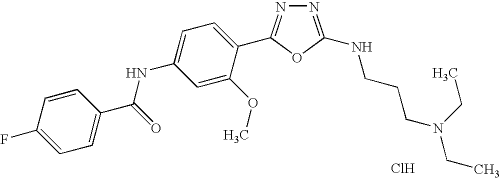 | N-[4-(5-{[3-(diethylamino)propyl]amino}-1,3,4-oxadiazol-2-yl)-3-(methyloxy)phenyl]-4-fluorobenzamide hydrochloride | 442 |
| 203(HCl) | 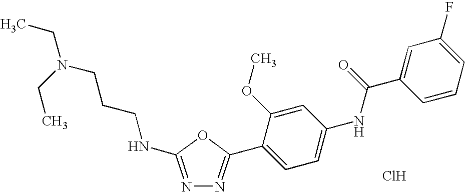 | N-[4-(5-{[3-(diethylamino)propyl]amino}-1,3,4-oxadiazol-2-yl)-3-(methyloxy)phenyl]-3-fluorobenzamide hydrochloride | 442 |
| 204(HCl) | 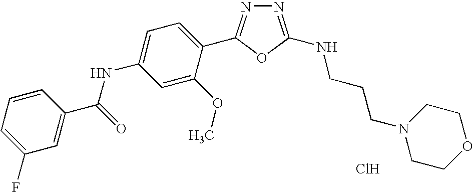 | 3-fluoro-N-[3-(methyloxy)-4-(5-{[3-(4-morpholinyl)propyl]amino}-1,3,4-oxadiazol-2-yl)phenyl]benzamidehydrochloride | 456 |
| 205(HCl) | 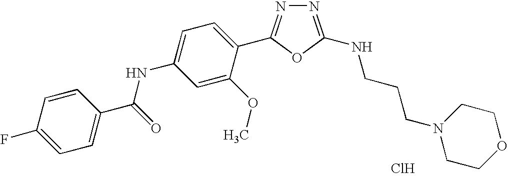 | 4-fluoro-N-[3-(methyloxy)-4-(5-{[3-(4-morpholinyl)propyl]amino}-1,3,4-oxadiazol-2-yl)phenyl]benzamidehydrochloride | 456 |

### General Procedure for Examples 206 to 237

To an Irori Minikan™ reactor was added 60 mg of resin-bound methyl-4-aminobenzoate functionalised resin. The reactor was treated sequentially with 1 mL of a solution of 1.25M diisopropylethylamine in dichloromethane and 1 mL of a solution of 1.0M acid chloride (for example, 3-(methyloxy)benzoyl chloride) in dichloromethane and allowed to shake at room temperature (20° C.) overnight (20 hours). The reactor was then washed sequentially with methanol and dichloromethane (2×) and treated with 2 mL of a solution of 0.7M potassium trimethylsilanoate (KOTMS) in tetrahydrofuran (THF) and allowed to shake at room temperature overnight. The reactor was again washed sequentially with methanol (2×), acetonitrile (1×), dichloromethane (1×), and acetonitrile (2×) and treated with 2 mL of a solution of 0.8M pyridine in N-methylpyrrolidinone (NMP). To this mixture was added 110 uL pentafluorophenyl trifluoroacetate (PFPTFA) and the mixture allowed to shake at room temperature overnight. The reactor was washed sequentially with NMP (1×), acetonitrile (2×), and dichloromethane (2×) and then treated with 2 mL of a solution of 0.3M hydrazine in NMP and allowed to shake at room temperature overnight. The reactor was again washed sequentially with NMP (1×), methanol (2×), dichloromethane (1×), and acetonitrile (1×) and then treated with 2 mL of a solution of 0.33M of the appropriate isothiocyanato compound in NMP and allowed to shake at room temperature overnight. The reactor was washed with NMP (1×), methanol (1×), dichloromethane (2×), and dimethylsulfoxide (DMSO) (1×) and treated with 2 mL of a solution of 0.4M N-(3-dimethylaminopropyl)-N′-ethylcarbodiimide hydrochloride (EDC) in DMSO and heated at 90° C. for 6 hours. The reactor was allowed to cool and washed with DMSO (1×) and methanol/dichloromethane (5×) to remove any soluble impurities prior to cleavage from the solid support. Cleavage from the support was achieved by treatment of the reactor with 2 mL of a solution of 10% trifluoroacetic acid (TFA) in dichloromethane. The resulting solution was evaporated to dryness to afford the final product.

Examples 206 to 237 were prepared in the manner described above. The compounds in Table 25 were isolated as TFA salts. Some Examples in Table 25 are the TFA salts of previously described Examples (denoted by Example No (TFA).

TABLE 25
Examples 206 to 237

| Example | | | |
|---|---|---|---|
| No. | Structure | Compound Name | [M + H]⁺ |
| 206 | 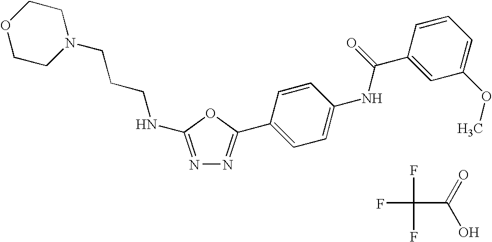 | 3-(methyloxy)-N-[4-(5-{[3-(4-morpholinyl)propyl]amino}-1,3,4-oxadiazol-2-yl)phenyl]benzamidetrifluoroacetate | 438 |
| 207 | 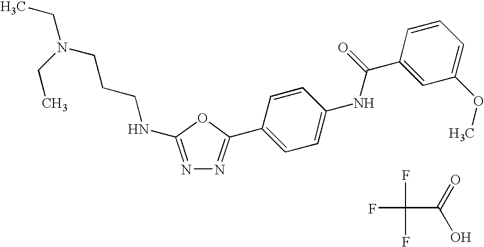 | N-[4-(5-{[3-(diethylamino)propyl]amino}-1,3,4-oxadiazol-2-yl)phenyl]-3-(methyloxy)benzamidetrifluoroacetate | 424 |
| 208 | 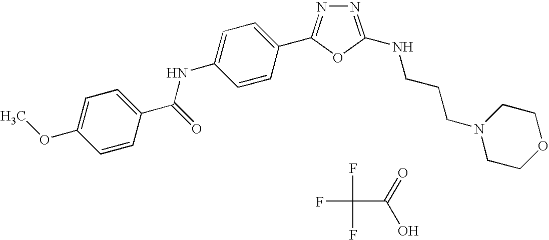 | 4-(methyloxy)-N-[4-(5-{[3-(4-morpholinyl)propyl]amino}-1,3,4-oxadiazol-2-yl)phenyl]benzamidetrifluoroacetate | 438 |
| 4(TFA) | 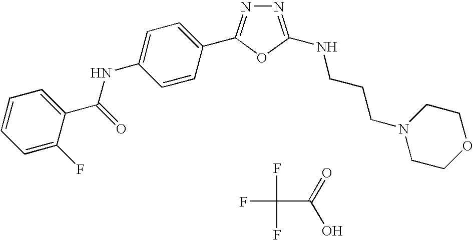 | 2-fluoro-N-[4-(5-{[3-(4-morpholinyl)propyl]amino}-1,3,4-oxadiazol-2-yl)phenyl]benzamidetrifluoroacetate | 426 |
| 151TFA) | 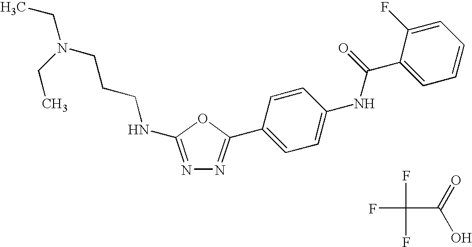 | N-[4-(5-{[3-(diethylamino)propyl]amino}-1,3,4-oxadiazol-2-yl)phenyl]-2-fluorobenzamidetrifluoroacetate | 412 |
| 195(TFA) | 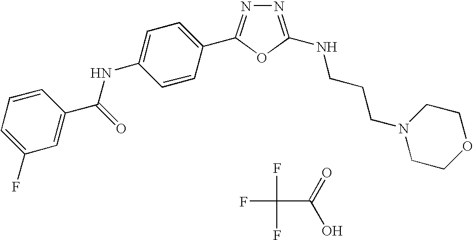 | 3-fluoro-N-[4-(5-{[3-(4-morpholinyl)propyl]amino}-1,3,4-oxadiazol-2-yl)phenyl]benzamidetrifluoroacetate | 426 |
| 209 | 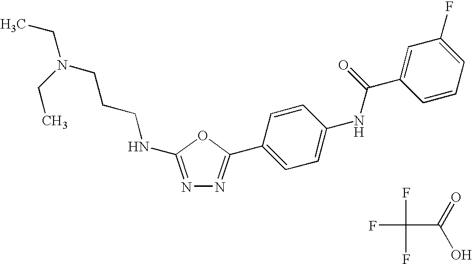 | N-[4-(5-{[3-(diethylamino)propyl]amino}-1,3,4-oxadiazol-2-yl)phenyl]-3-fluorobenzamidetrifluoroacetate | 412 |
| 201(TFA) | 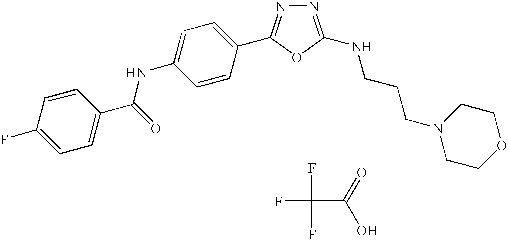 | 4-fluoro-N-[4-(5-{[3-(4-morpholinyl)propyl]amino}-1,3,4-oxadiazol-2-yl)phenyl]benzamidetrifluoroacetate | 426 |
| 210 | 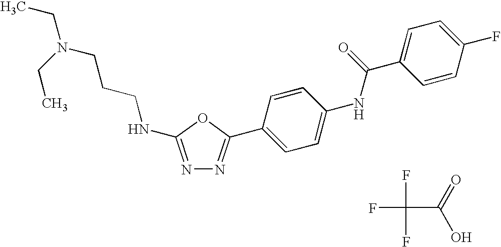 | N-[4-(5-{[3-(diethylamino)propyl]amino}-1,3,4-oxadiazol-2-yl)phenyl]-4-fluorobenzamidetrifluoroacetate | 412 |
| 194(TFA) | 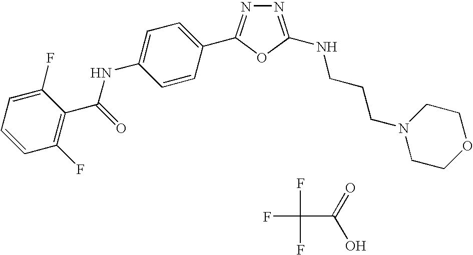 | 2,6-difluoro-N-[4-(5-{[3-(4-morpholinyl)propyl]amino}-1,3,4-oxadiazol-2-yl)phenyl]benzamidetrifluoroacetate | 444 |
| 211 | 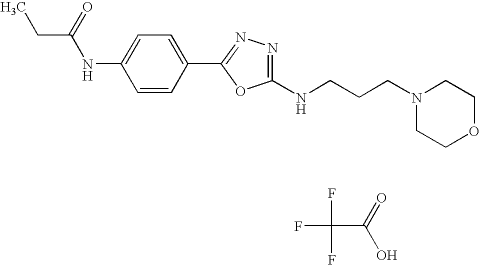 | N-[4-(5-{[3-(4-morpholinyl)propyl]amino}-1,3,4-oxadiazol-2-yl)phenyl]propanamidetrifluoroacetate | 360 |
| 212 | 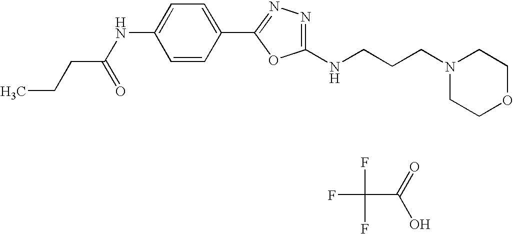 | N-[4-(5-{[3-(4-morpholinyl)propyl]amino}-1,3,4-oxadiazol-2-yl)phenyl]butanamidetrifluoroacetate | 374 |
| 213 | 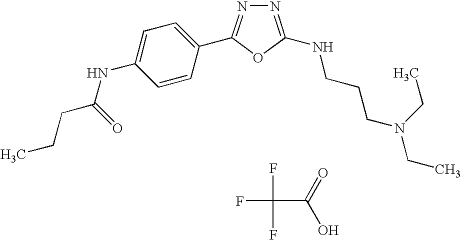 | N-[4-(5-{[3-(diethylamino)propyl]amino}-1,3,4-oxadiazol-2-yl)phenyl]butanamidetrifluoroacetate | 360 |
| 214 | 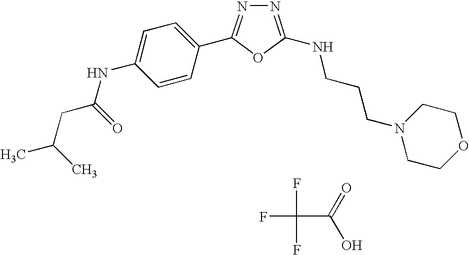 | 3-methyl-N-[4-(5-{[3-(4-morpholinyl)propyl]amino}-1,3,4-oxadiazol-2-yl)phenyl]butanamidetrifluoroacetate | 388 |
| 215 | 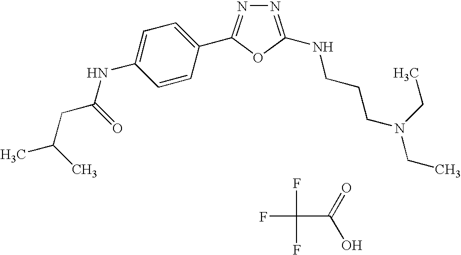 | N-[4-(5-{[3-(diethylamino)propyl]amino}-1,3,4-oxadiazol-2-yl)phenyl]-3-methylbutanamidetrifluoroacetate | 374 |
| 216 | 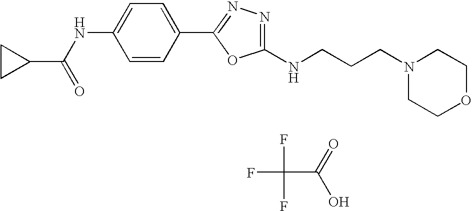 | N-[4-(5-{[3-(4-morpholinyl)propyl]amino}-1,3,4-oxadiazol-2-yl)phenyl]cyclopropane-carboxamidetrifluoroacetate | 372 |
| 217 | 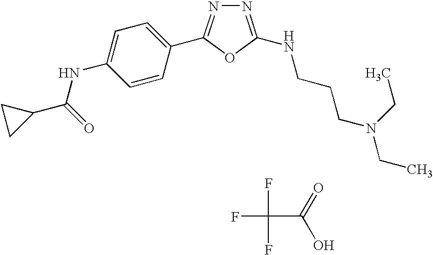 | N-[4-(5-{[3-(diethylamino)propyl]amino}-1,3,4-oxadiazol-2-yl)phenyl]cyclopropane-carboxamide trifluoroacetate | 358 |
| 218 | 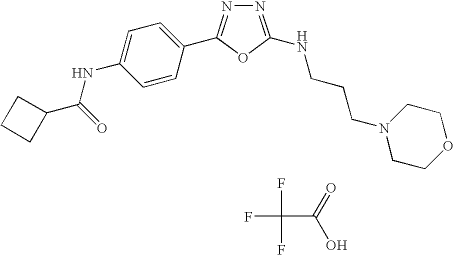 | N-[4-(5-{[3-(4-morpholinyl)propyl]amino}-1,3,4-oxadiazol-2-yl)phenyl]cyclobutane-carboxamide trifluoroacetate | 386 |
| 219 | 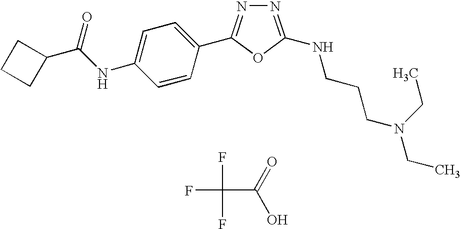 | N-[4-(5-{[3-(diethylamino)propyl]amino}-1,3,4-oxadiazol-2-yl)phenyl]cyclobutane-carboxamide trifluoroacetate | 372 |
| 220 | 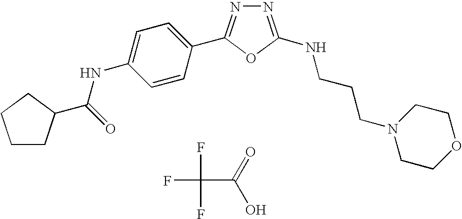 | N-[4-(5-{[3-(4-morpholinyl)propyl]amino}-1,3,4-oxadiazol-2-yl)phenyl]cyclopentane-carboxamide trifluoroacetate | 400 |
| 221 | 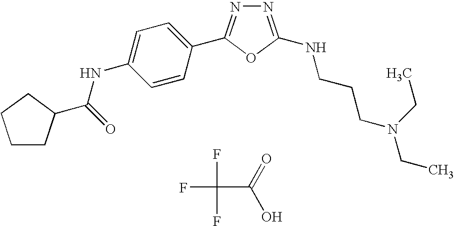 | N-[4-(5-{[3-(diethylamino)propyl]amino}-1,3,4-oxadiazol-2-yl)phenyl]cyclopentane-carboxamide trifluoroacetate | 386 |
| 222 | 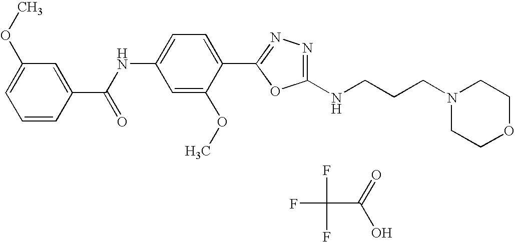 | 3-(methyloxy)-N-[3-(methyloxy)-4-(5-{[3-(4-morpholinyl)propyl]amino}-1,3,4-oxadiazol-2-yl)phenyl]benzamidetrifluoroacetate | 468 |
| 223 | 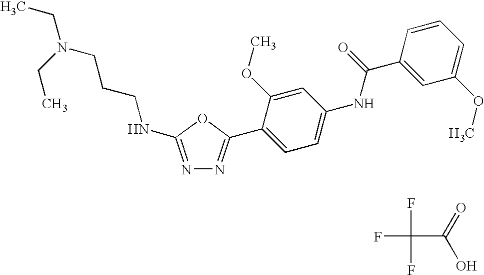 | N-[4-(5-{[3-(diethylamino)propyl]amino}-1,3,4-oxadiazol-2-yl)-3-(methyloxy)phenyl]-3-(methyloxy)benzamidetrifluoroacetate | 454 |
| 224 | 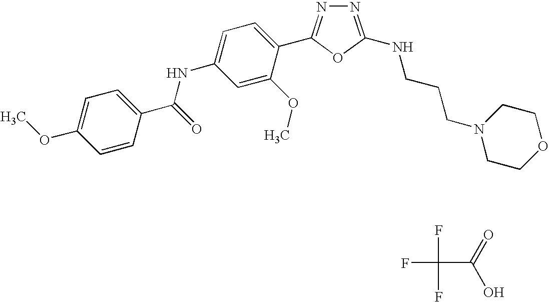 | 4-(methyloxy)-N-[3-(methyloxy)-4-(5-{[3-(4-morpholinyl)propyl]amino}-1,3,4-oxadiazol-2-yl)phenyl]benzamidetrifluoroacetate | 468 |
| 225 | 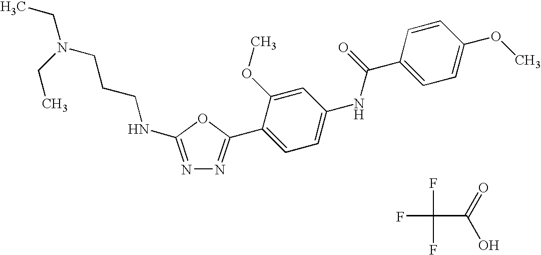 | N-[4-(5-{[3-(diethylamino)propyl]amino}-1,3,4-oxadiazol-2-yl)-3-(methyloxy)phenyl]-4-(methyloxy)benzamidetrifluoroacetate | 454 |
| 3(TFA) | 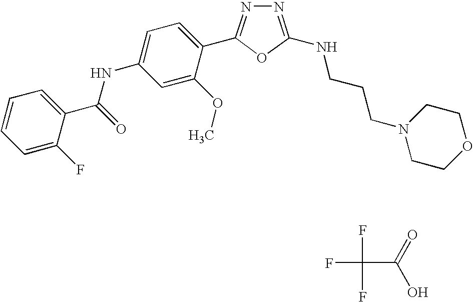 | 2-fluoro-N-[3-(methyloxy)-4-(5-{[3-(4-morpholinyl)propyl]amino}-1,3,4-oxadiazol-2-yl)phenyl]benzamidetrifluoroacetate | 456 |
| 2(TFA) | 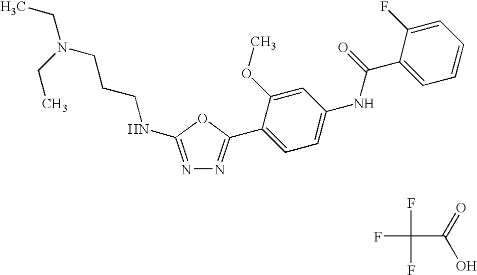 | N-[4-(5-{[3-(diethylamino)propyl]amino}-1,3,4-oxadiazol-2-yl)-3-(methyloxy)phenyl]-2-fluorobenzamidetrifluoroacetate | 442 |
| 204(TFA) | 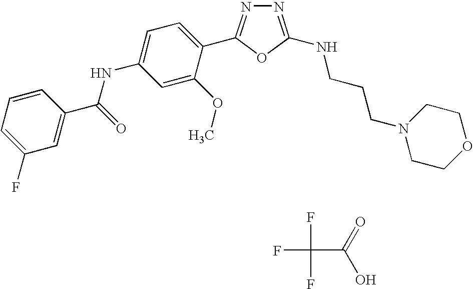 | 3-fluoro-N-[3-(methyloxy)-4-(5-{[3-(4-morpholinyl)propyl]amino}-1,3,4-oxadiazol-2-yl)phenyl]benzamidetrifluoroacetate | 456 |
| 203(TFA) | 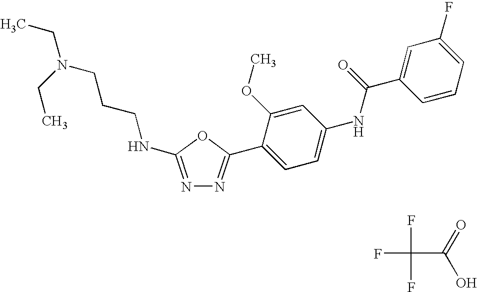 | N-[4-(5-{[3-(diethylamino)propyl]amino}-1,3,4-oxadiazol-2-yl)-3-(methyloxy)phenyl]-3-fluorobenzamidetrifluoroacetate | 442 |
| 205(TFA) | 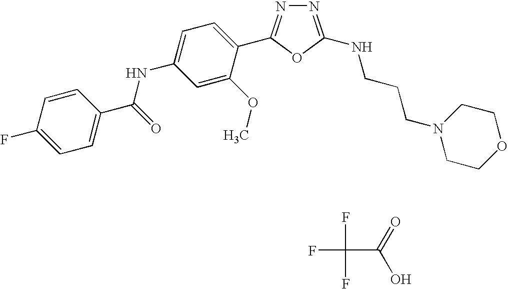 | 4-fluoro-N-[3-(methyloxy)-4-(5-{[3-(4-morpholinyl)propyl]amino}-1,3,4-oxadiazol-2-yl)phenyl]benzamidetrifluoroacetate | 456 |
| 202(TFA) | 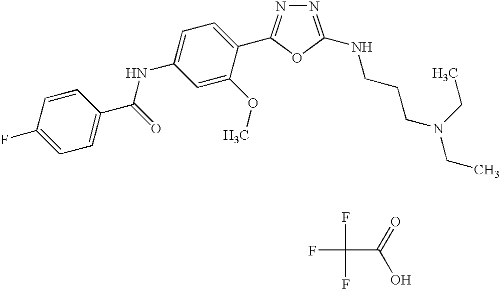 | N-[4-(5-{[3-(diethylamino)propyl]amino}-1,3,4-oxadiazol-2-yl)-3-(methyloxy)phenyl]-4-fluorobenzamidetrifluoroacetate | 442 |
| 226 | 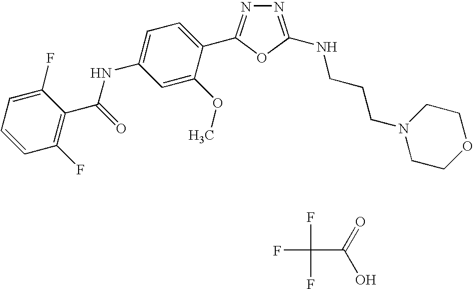 | 2,6-difluoro-N-[3-(methyloxy)-4-(5-{[3-(4-morpholinyl)propyl]amino}-1,3,4-oxadiazol-2-yl)phenyl]benzamidetrifluoroacetate | 474 |
| 197(TFA) | 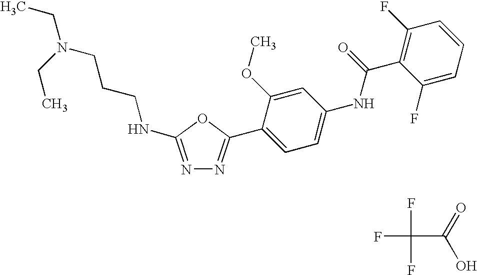 | N-[4-(5-{[3-(diethylamino)propyl]amino}-1,3,4-oxadiazol-2-yl)-3-(methyloxy)phenyl]-2,6-difluorobenzamidetrifluoroacetate | 460 |
| 227 | 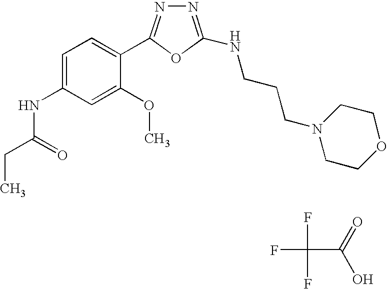 | N-[3-(methyloxy)-4-(5-{[3-(4-morpholinyl)propyl]amino}-1,3,4-oxadiazol-2-yl)phenyl]propanamidetrifluoroacetate | 390 |
| 228 | 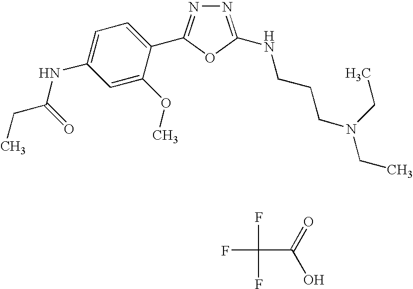 | N-[4-(5-{[3-(diethylamino)propyl]amino}-1,3,4-oxadiazol-2-yl)-3-(methyloxy)phenyl]propanamidetrifluoroacetate | 376 |
| 199(TFA) | 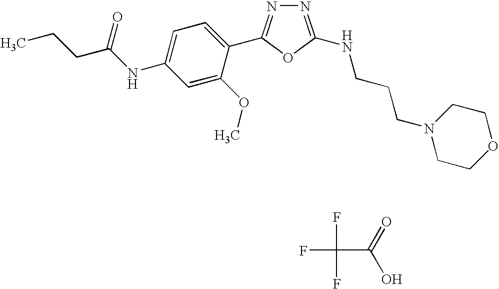 | N-[3-(methyloxy)-4-(5-{[3-(4-morpholinyl)propyl]amino}-1,3,4-oxadiazol-2-yl)phenyl]butanamidetrifluoroacetate | 404 |
| 200(TFA) | 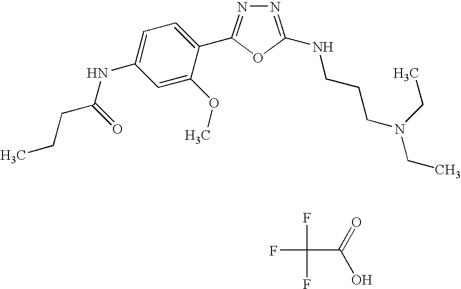 | N-[4-(5-{[3-(diethylamino)propyl]amino}-1,3,4-oxadiazol-2-yl)-3-(methyloxy)phenyl]butanamidetrifluoroacetate | 390 |
| 229 | 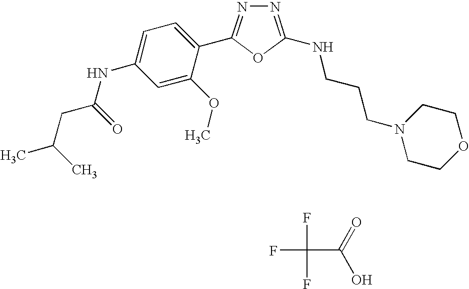 | 3-methyl-N-[3-(methyloxy)-4-(5-{[3-(4-morpholinyl)propyl]amino}-1,3,4-oxadiazol-2-yl)phenyl]butanamidetrifluoroacetate | 418 |
| 230 | 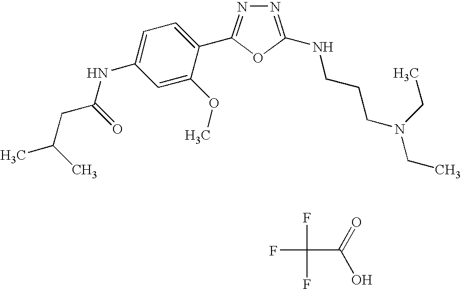 | N-[4-(5-{[3-(diethylamino)propyl]amino}-1,3,4-oxadiazol-2-yl)-3-(methyloxy)phenyl]-3-methylbutanamidetrifluoroacetate | 404 |
| 198(TFA) | 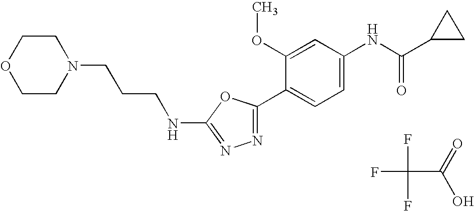 | N-[3-(methyloxy)-4-(5-{[3-(4-morpholinyl)propyl]amino}-1,3,4-oxadiazol-2-yl)phenyl]cyclopropane-carboxamidetrifluoroacetate | 402 |
| 231 | 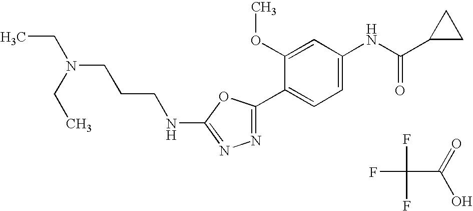 | N-[4-(5-{[3-(diethylamino)propyl]amino}-1,3,4-oxadiazol-2-yl)-3-(methyloxy)phenyl]cyclopro-panecarboxamidetrifluoroacetate | 388 |
| 232 | 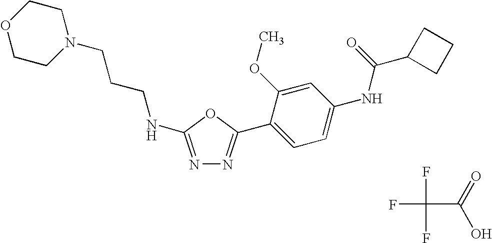 | N-[3-(methyloxy)-4-(5-{[3-(4-morpholinyl)propyl]amino}-1,3,4-oxadiazol-2-yl)phenyl]cyclobutane-carboxamide trifluoroacetate | 416 |
| 233 | 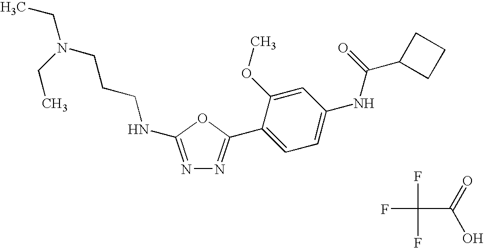 | N-[4-(5-{[3-(diethylamino)propyl]amino}-1,3,4-oxadiazol-2-yl)-3-(methyloxy)phenyl]cyclo-butanecarboxamidetrifluoroacetate | 402 |
| 234 | 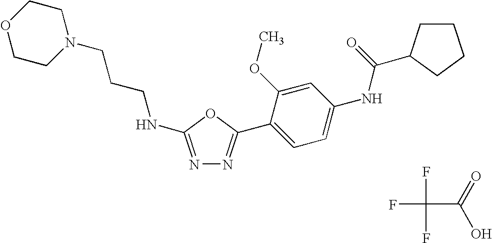 | N-[3-(methyloxy)-4-(5-{[3-(4-morpholinyl)propyl]amino}-1,3,4-oxadiazol-2-yl)phenyl]cyclopentane-carboxamidetrifluoroacetate | 430 |
| 235 | 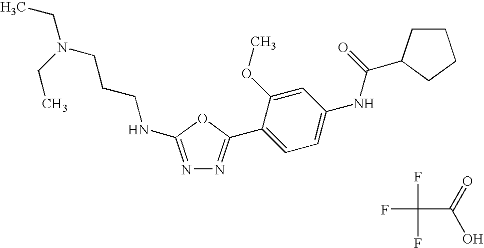 | N-[4-(5-{[3-(diethylamino)propyl]amino}-1,3,4-oxadiazol-2-yl)-3-(methyloxy)phenyl]cyclo-pentanecarboxamidetrifluoroacetate | 416 |
| 236 | 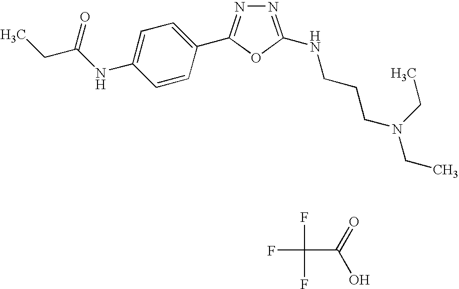 | N-[4-(5-{[3-(diethylamino)propyl]amino}-1,3,4-oxadiazol-2-yl)phenyl]propanamidetrifluoroacetate | |
| 237 | 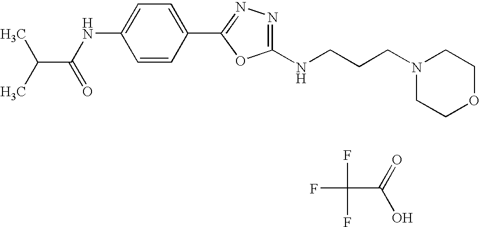 | 2-methyl-N-[4-(5-{[3-(4-morpholinyl)propyl]amino}-1,3,4-oxadiazol-2-yl)phenyl]propanamidetrifluoroacetate | |

### Example 25A

### Alternative Route to 2-fluoro-N-[3-fluoro-4-(5-{[4-(4-morpholinyl)butyl]amino}-1,3,4-oxadiazol-2-yl)phenyl]benzamide

### 4-(4-Isothiocyanatobutyl)morpholine

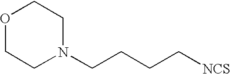

[4-(4-Morpholinyl)butyl]amine (1.1 g 7 mmol) dissolved in THF (10 mL) was treated with 1′1 thiocarbonyldiimidazole (1.18 g, 6.65 mmol) and stirred at room temperature for 3 hrs. The solution was used directly in the next step.

### 2-Fluoro-N-(3-fluoro-4-{[2-({[4-(4-morpholinyl)butyl]amino}carbonothioyl)hydrazino]carbonyl}phenyl)benzamide

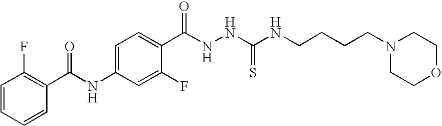

2-Fluoro-N-[3-fluoro-4-(hydrazinocarbonyl)phenyl]benzamide (1.02 g, 3.5 mmol) was stirred in THF (3 mL) and treated with a solution of the 4-(4-isothiocyanatobutyl)morpholine (4.65 mmol) in THF (7 mL) and heated at 60° C. for 5 hours. A yellow solution formed. A further (0.9 mL) of the 4-(4-isothiocyanatobutyl)morpholine solution was added and the solution stirred overnight at room temperature. A further (0.6 mL) of the 4-(4-isothiocyanatobutyl)morpholine solution was added and the solution heated at 60° C. for 2 hours. The suspension was cooled, the solid filtered, washed with ether and dried to yield the title compound (1.7 g). This solid was used without further purification.

### 2-Fluoro-N-[3-fluoro-4-(5-{[4-(4-morpholinyl)butyl]amino}-1,3,4-oxadiazol-2-yl)phenyl]benzamide

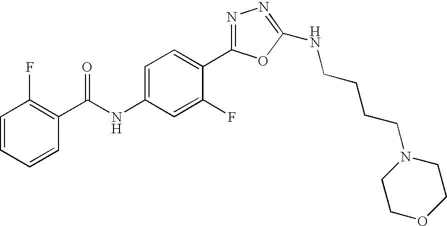

A solution of 2-fluoro-N-(3-fluoro-4-{[2-({[4-(4-morpholinyl)butyl]amino}carbonothioyl)hydrazino]carbonyl}phenyl)benzamide (1.7 g crude) and N-(3-dimethylaminopropyl)-N′-ethylcarbodiimide hydrochloride (793 mg, 4.15 mmol) in DMF (20 mL) was heated at 80° C. for 5 hours then cooled and diluted with ethyl acetate. The solution was washed with water (3×) dried (magnesium sulphate) and evaporated. Trituration with ether followed by acetone yielded the title compound (780 mg) as a pale yellow solid. LC/MS (ES+ve): [M+H]⁺ at m/z 458.1 (C₂₃H₂₅F₂N₅O₂) requires [M+H]⁺ at m/z 458).

### Example 131A

### Alternative Route to Example 131

### 2-Fluoro-N-[4-(5-{[4-(4-fluoro-1-piperidinyl)butyl]amino}-1,3,4-oxadiazol-2-yl)phenyl]benzamide

### 4-(4-Fluoro-1-piperidinyl)butyl]amine

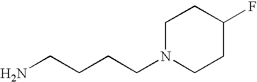

4-Bromobutylpthalimide (7.0 g, 24.8 mmol), triethylamine (10.4 mL, 74.4 mmol) and 4-fluoropiperidine hydrochloride (4.50 g, 32.3 mmol) were added to ethanol (70 mL) and the mixture heated to reflux for 6 h before allowed to cool to room temperature overnight. Hydrazine hydrate (2.86 g, 57.0 mmol) was then added and the reaction heated to reflux for 45 min. The solid was filtered off and the solution concentrated in vacuo. Diethyl ether was added and the solid formed was filtered off. The solid from the second batch was separated between dichloromethane and aqueous sodium hydroxide (2M). The DCM layer was washed again with aqueous sodium hydroxide (2M) and the organic solution was isolated and the solvent removed in vacuo to give a colourless oil. The first batch of solid was added to the aqueous layer and extracted with dichloromethane then chloroform:isopropyl alcohol (4:1). Once isolated and the solvent removed in vacuo all batches of product from the separations were combined (2.37 g) to give a colourless oil.

### Methyl 4-{[(2-fluorophenyl)carbonyl]amino}benzoate

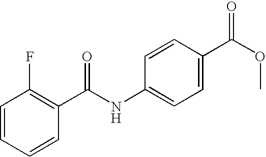

To a suspension of methyl 4-aminobenzoate (15.0 g, 99.0 mmol) in dichloromethane (120 mL) at 0° C. under a flush of argon was added diisopropylamine, (17.2 mL, 99.0 mmol) followed by 2-fluorobenzoyl chloride (12.9 mL, 109 mmol). After 5 minutes the cooling bath was removed and the solution stirred for 4 h. The reaction mixture was filtered to remove a precipitate and the filtrate washed with aqueous hydrochloric acid (2M, 100 mL then 70 mL), dried (phase separator) and the solvent removed in vacuo to give the title compound (24.6 g) as an off-white solid. LC/MS (ES+ve): [M+H]⁺ at m/z 274 (C₁₅H₁₂FNO₃ requires [M+H]⁺ at m/z 274.

### 2-Fluoro-N-[4-(hydrazinocarbonyl)phenyl]benzamide

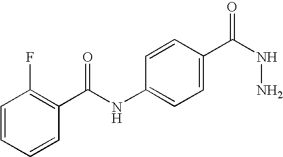

To a mixture of methyl 4-{[(2-fluorophenyl)carbonyl]amino}benzoate (24.6 g, 90.0 mmol) in methanol (400 mL) was added hydrazine hydrate (30.5 mL, 630 mmol) and the reaction heated to reflux overnight. Further hydrazine hydrate (15 mL, 215 mmol) was added and the reaction heated for a further 24 h. The title compound was isolated from the reaction mixture by filtration, washed with methanol and dried in a vacuum oven over the weekend to give the title compound (16.7 g). LC/MS (ES+ve): [M+H]⁺ at m/z 274 (C₁₄H₁₂FN₃O₂ requires [M+H]⁺ at m/z 274.

### 2-Fluoro-N-[4-(5-{[4-(4-fluoro-1-piperidinyl)butyl]amino}-1,3,4-oxadiazol-2-yl)phenyl]benzamide

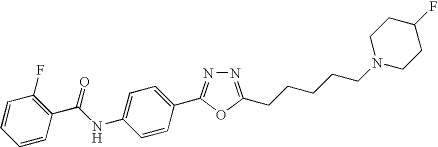

To a solution of 1,1-thiocarbonyldiimidazole (1.63 g, 9.17 mmol) in N,N-dimethylformamide (80 mL) was added [4-(4-fluoro-1-piperidinyl)butyl]amine (1.60 g, 9.17 mmol) and the reaction stirred at room temperature under a flush of argon for 20 h. 2-Fluoro-N-[4-(hydrazinocarbonyl)phenyl]benzamide (2.50 g, 9.17 mmol) was then added and the reaction stirred for a further 24 h at room temperature. N-[3-(dimethylamino)propyl]-N′-ethylcarbodiimide hydrochloride (3.50 g, 18.3 mmol) was then added and the reaction heated at 65° C. for 4 h. The reaction was not complete so it was heated at 65° C. overnight before addition of further N-[3-(dimethylamino)propyl]-N′-ethylcarbodiimide hydrochloride (1.76 g, 9.17 mmol) and the reaction heated for 24 h. The reaction mixture was concentrated in vacuo and separated between ethyl acetate (100 mL) and aqueous sodium hydroxide (2M, 100 mL). The organic phase was washed with further sodium hydroxide (2M, 100 mL) and water (2×70 mL) before it was seperated and concentrated in vacuo. The solid was triturated with acetone and the resultant solid was dried in a vacuum oven overnight. It was then recrystallised from ethanol to give the title compound (1.05 g) as a white solid. LC/MS (ES+ve): [M+H]⁺ at m/z 456 (C₂₄H₂₇F₂N₅O₂ requires [M+H]⁺ at m/z 456.

### Alternative Route to Example 26

### 2-Fluoro-N-[3-fluoro-4-(5-{[4-(4-fluoro-1-piperidinyl)butyl]amino}-1,3,4-oxadiazol-2-yl)phenyl]benzamide

### 4-Amino-2-fluorobenzoic Acid

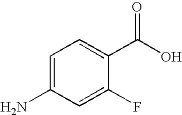

A solution of 2-fluoro-4-nitrobenzoic acid (25 g, 135 mmol) in ethanol (500 mL) was hydrogenated over 10% palladium on charcoal catalyst overnight. The catalyst was filtered and the filtrate evaporated to dryness to give the title compound (20.3 g) as a cream solid. LC/MS (ES+ve): [M+H]⁺ at m/z 156 (C₇H₆FNO₂ requires [M+H]⁺ at m/z 156).

A second smaller batch was prepared in a similar manner to produce 4.1 g of title compound.

### Methyl 4-amino-2-fluorobenzoate

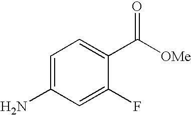

Concentrated sulphuric acid (48 mL) was added dropwise to a stirred solution of 4-amino-2-fluorobenzoic acid (24.4 g) in methanol (480 mL) and heated under reflux for 3 hours. The methanol was evaporated and the residue partitioned between water (600 mL) and ethyl acetate (500 ml). The mixture was made basic with solid potassium carbonate and the organic layer separated. The organic layer was dried (magnesium sulphate) and evaporated to give the title compound (25.5 g) as a yellow solid.

LC/MS (ES+ve): [M+H]⁺ at m/z 170 (C₈H₈FNO₂ requires [M+H]⁺ at m/z 170)

### Methyl 2-fluoro-4-{[(2-fluorophenyl)carbonyl]amino}benzoate

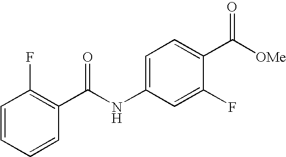

2-Fluorobenzoyl chloride (19.8 mL 166 mmol) was added dropwise to a stirred solution of methyl 4-amino-2-fluorobenzoate (25.5 g, 151 mmol) and triethylamine (23 mL 166 mmol) in DCM (250 mL) and cooled in an icebath. The solution was stirred for 1 hour at room temperature, washed with 2M HCl (300 mL), water (200 mL) dried (magnesium sulphate) and evaporated. The residue was triturated with ether to give the title compound (35.7 g) as a yellow solid. LC/MS (ES+ve): [M+H]⁺ at m/z 292 (C₁₅H₁₁F₂NO₃ requires [M+H]⁺ at m/z 292)

### 2-Fluoro-N-[3-fluoro-4-(hydrazinocarbonyl)phenyl]benzamide

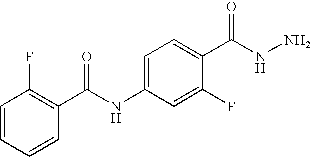

A mixture of methyl 2-fluoro-4-{[(2-fluorophenyl)carbonyl]amino}benzoate (10 g, 34 mmol), hydrazine hydrate (8.34 mL, 172 mmol) and methanol (100 mL) was heated under reflux overnight, cooled and filtered. The solid was washed with methanol and ether and dried to give the title compound (7.7 g) as a white solid. LC/MS (ES+ve): [M+H]⁺ at m/z 292 (C₁₄H₁₁F₂N₃O₂ requires [M+H]⁺ at m/z 292)

### [4-(4-Fluoro-1-piperidinyl)butyl]amine

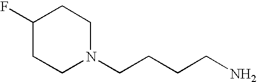

A mixture of N-(4-bromobutyl)phthalimide (31 g, 110 mmol), 4-fluoropiperidine hydrochloride (20 g, 143 mmol) and triethylamine (45.7 mL, 329 mmol) in ethanol (300 mL) was heated under reflux for 6 hours and allowed to cool to room temperature overnight. Hydrazine hydrate (12.3 mL, 253 mmol) was added and the reaction was heated under reflux for 1 hours then allowed to cool to room temperature. The precipitated solid was filtered washed with ethanol and the filtrate evaporated to dryness. The residue was dissolved in 2M aqueous sodium hydroxide and ethyl acetate and the organic layer was dried (magnesium sulphate) and evaporated to give the title compound (9.3 g) as a light brown oil.

### 4-Fluoro-1-(4-isothiocyanatobutyl)piperidine

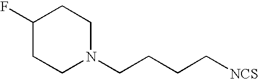

[4-(4-Morpholinyl)butyl]amine (6.96 g 40 mmol) dissolved in THF (75 mL) was treated with 1′1 thiocarbonyldiimidazole (7.12 g, 40 mmol) and stirred at room temperature for 3 hours. The solution was washed with water, dried (magnesium sulphate) and evaporated to give the title compound (12 g) as a brown oil, which was used without further purification.

### 2-Fluoro-N-(3-fluoro-4-{[2-({[4-(4-fluoro-1-piperidinyl)butyl]amino}carbonothioyl)hydrazino]carbonyl}phenyl)benzamide

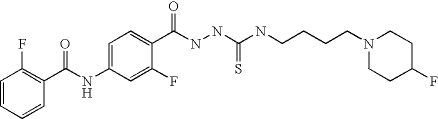

A suspension of 2-fluoro-N-[3-fluoro-4-(hydrazinocarbonyl)phenyl]benzamide (4 g, 13.7 mmol) and 4-fluoro-1-(4-isothiocyanatobutyl)piperidine (4.15 g, 20.5 mmol) in tetrahydrofuran (45 mL) was heated at 60° C. for 5 hours. A further (800 mg, 4 mmol) of 4-fluoro-1-(4-isothiocyanatobutyl)piperidine was added and the solution heated at 60° C. for 3.5 hours then stirred overnight at room temperature. The solvent was evaporated, the residue triturated with ether, filtered and dried to yield the title compound (7.5 g) as a white solid. [M+H]⁺ at m/z 508 (C₂₄H₂₈F₃N₅O₂S) requires [M+H]⁺ at m/z 508). This solid was used without further purification.

### 2-Fluoro-N-[3-fluoro-4-(5-{[4-(4-fluoro-1-piperidinyl)butyl]amino}-1,3,4-oxadiazol-2-yl)phenyl]benzamide

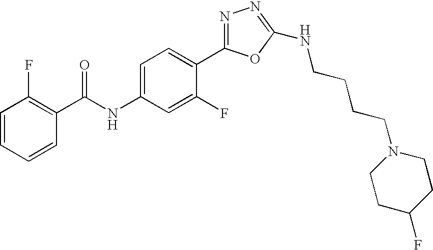

A solution of 2-fluoro-N-(3-fluoro-4-{[2-({[4-(4-fluoro-1-piperidinyl)butyl]amino}carbonothioyl)hydrazino]carbonyl}phenyl)benzamide

(7.5 g crude) and N-(3-dimethylaminopropyl)-N′-ethylcarbodiimide hydrochloride (3.14 g, 16.4 mmol) in dimethylformamide (75 mL) was heated at 80° C. for 4 hours then cooled and diluted with ethyl acetate. The solution was washed with water (3×) dried (magnesium sulphate) and evaporated. The solid was triturated with acetone, filtered and dried. The solid was dissolved in 2% methanol/dichloromethane (120 mL), washed with water (4×30 mL) to remove residual dimethylformamide, dried (magnesium sulphate) and evaporated. Trituration with ether yielded the title compound (2.66 g) as a white solid.

LC/MS (ES+ve): [M+H]⁺ at m/z 474.0 (C₂₄H₂₆F₃N₅O₂) requires [M+H]⁺ at m/z 474).

It is to be understood that the present invention covers all combinations of particular and preferred subgroups described herein above.

### Assays for Determining Biological Activity

α7 nAChR FLIPR® (Fluorometric Imaging Plate Reader) Assay

Function of the heterologous expressed α7 nAChR was assessed by a FLIPR-Ca²⁺ assay. Since nAChRs are non-selective cation channels with high permeability to Ca²⁺, these studies were carried out by measuring changes of intracellular Ca²⁺ concentration using the Ca²⁺-chelating fluorescent dye Fluo-4 and FLIPR® (Fluorometric Imaging Plate Reader) technology.

GH4C1cells (pituitary tumor, immortalized cell line) stably transfected with human α7 nAChR (Biocat ID 96986), were thawed, suspended in growth medium (Ham's Nutrient Mixture F10—Ham's F10, Invitrogen 31550-023, 15% Horse Serum heat inactivated—Invitrogen 26050-047, 2.5% Foetal Bovine Serum—FBS, Gibco 10500-064, 200 μg/ml Hygromycin B—Invitrogen, 10687-010, 10 mg/L Phenol Red—Sigma, P 0290, 1 mM Glutamine—Invitrogen, 25030-024) and plated in 500 cm² Triple Flask.

72 hours before an experiment, cells growing in suspension were harvested, centrifuged, resuspended in growth medium at a density of 1.8×10⁵/mL and plated in coated clear bottom black 384 wells plates (Pierce) at 9000 cells/well. Cells were then incubated at 30° C., 5% CO₂ for 72 hours.

On the day of the experiment, cells were washed twice with Assay Buffer (AB) (145 mM NaCl, 5 mM KCl, 1 mM MgCl₂, 2 mM CaCl₂, 20 mM HEPES, 5.5 mM Glucose pH=7.3) containing 2.5 mM Probenecid. Changes in the intracellular Ca²⁺ content of stably transfected cells were measured using the Ca²⁺ chelating dye Fluo-4 (Tef Labs 0152) in conjunction with a FLIPR® (Molecular Devices). The cell permeant dye Fluo-4 was prepared to a concentration of 1 mM in 100% DMSO and 10% Pluronic acid. The dye was then diluted with AB to a final concentration of 2 μM and placed on the cells. After 45-60 minutes dye loading incubation at 37° C., the unincorporated dye was removed from the cells by washing (80 μL, 3 times) with AB, and a final volume of 30 μL/well of AB was left in each well.

Plates containing test compounds (dissolved in 100% DMSO at 2 mM and serially diluted with DMSO) were copied into “daughter” plates (1 μL/well dispensation). Just prior to starting the assay, the “daughter” plate was diluted with 50 μL/well of AB.

The plates were then placed in the FLIPR®, and cell fluorescence was determined before drug addition (30 seconds) and monitored (excitation 488 nm, emission 510-580 nm) immediately following exposure to compounds. Maximum fluorescence values were recorded and fitted for agonist EC50 calculations.

### Results

The compounds of Examples 1-237 were tested in the α7 nAChR FLIPR® assay except Example 134. Examples 194 to 205 were tested as the HCl salt.

The results are expressed as pEC50 values. A pEC50 is the negative logarithm of the agonist EC50 calculation as determined in the α7 nAChR FLIPR® assay. Certain Examples have been tested more than once. Variations in pEC50 may arise between tests.

The compounds of Examples 1-237 exhibited a pEC50 value 25.

The compounds of Examples 1-14, 16 to 19, 21, 23 25-29, 31-35, 37, 40-48, 50-56, 58-64, 66-108, 110-129, 131-132, 135-137, 140, 144-161, 163-165, 167-177, 181, 183, 185, 188-191, 193-195, 199-200, 202-204, 209, 225, 226, 229-231, 234, 235. exhibited a pEC50 value ≧6.

More particularly, the compounds of Examples 1, 5, 6, 26, 31-34, 44-46, 48, 52, 54, 55, 62, 71, 73, 75, 76, 78, 83, 85, 89, 90, 92, 94, 96, 98, 100, 102, 104, 106, 108, 112, 114, 115, 119-125, 127-128, 145, 147, 152-154, 156, 158, 164, 169-173, 176, 190 exhibited a pEC50 value ≧7.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I); 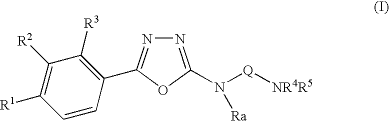
wherein
Q represents —(CH₂)ₙ— wherein n represents 3 or 4;
Ra represents hydrogen or CH₃;
R¹ represents —NR⁶COR⁷;
R² represents hydrogen, halogen or C₁₋₆alkyl;
R³ represents hydrogen, C₁₋₆alkyl, C₁₋₆alkoxy, halo, haloC₁₋₆alkyl, or —OhaloC₁₋₆alkyl;
R⁴ and R⁵ independently represent hydrogen, C₁₋₆ alkyl, C₃₋₆cycloalkyl, or a 5 to 6 membered heterocyclic ring which is unsubstituted or substituted with 1, 2, or 3 substituents selected from methyl or fluoro;
or R⁴ and R⁵ together with the nitrogen atom to which they are attached form a nitrogen containing heterocyclyl group which is unsubstituted or substituted with 1, 2, or 3 substituents selected from methyl or fluoro;
R⁶ represents hydrogen;
R⁷ represents C₁₋₅alkyl, C₃₋₈ cycloalkyl, tetrahydropyranyl, thiophenyl, pyridyl, oxazolyl, isoxazolyl, furanyl, —(CH₂)ₘaryl wherein the furanyl, thiophenyl, pyridyl, oxazolyl, isooxazolyl or aryl can be unsubstituted or substituted by one to three substitutents selected from halo, methyl, CF₃, OCF₃ or OMe wherein OMe is not present at the position ortho to the carbonyl and the CF₃ is not at the position meta or para to the carbonyl;
m represents 0 or 1;
with the proviso that R⁷ is not isopropyl;
or a salt thereof.

2. A compound according to claim 1 wherein Ra represents hydrogen.

3. A compound according to claim 1 wherein R² represents hydrogen.

4. A compound according to claim 1 wherein R³ represents hydrogen, chloro, fluoro, CF₃, —OCH₃ or ethyl.

5. A compound according to claim 1 wherein R⁴ and R⁵ together with the nitrogen atom which they are attached form an nitrogen containing heterocyclyl group which is unsubstituted or substituted with 1, 2, or 3 substituents selected from methyl or fluoro.

6. A compound according to claim 1 wherein R⁷ represents a cyclohexyl group or R⁷ represents aryl unsubstituted or substituted by one two or three halogen atoms.

7. A compound of formula (Ia): 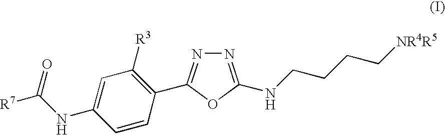
wherein
R³ represents hydrogen, chloro, fluoro, CF₃, OCH₃ or ethyl;
R⁴ and R⁵ together with the nitrogen atom to which they are attached form a nitrogen containing heterocyclyl group which is unsubstituted or substituted with 1, 2, or 3 substituents selected from methyl or fluoro;
R⁷ represents a cyclohexyl group or a phenyl which can be unsubstituted or substituted by one to three halo substitutents;
or a salt thereof.

8. A compound according to claim 1 wherein the salt is a pharmaceutically acceptable salt.

9. (canceled)

10. A pharmaceutical composition comprising a compound according to claim 1 or a pharmaceutically acceptable salt thereof together with a pharmaceutical carrier and/or excipient.

11-12. (canceled)

13. A method of treating a human or animal subject suffering from a condition selected from neurological diseases, psychiatric disorders, pain related disorders, obesity, sepsis, and gastro-intestinal disorder, which comprises administering to said subject an effective amount of a compound according to claim 1 or a pharmaceutically acceptable salt thereof.

14-15. (canceled)

16. A method of claim 13, wherein the subject is a human.

17. A compound of formula 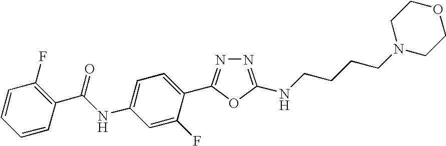
or a salt thereof.
